(19) United States
(12) Patent Application Publication
(10) Pub. No.: US 2026/0227696 A1
(43) Pub. Date: Aug. 6, 2026

(54) RESIST COMPOSITION, RESIST PATTERN FORMATION METHOD, COMPOUND, AND POLYMER COMPOUND

(71) Applicant: TOKYO OHKA KOGYO CO., LTD., Kawasaki-shi (JP)

(72) Inventors: Koshi ONISHI, Kawasaki-shi (JP); Takashi NAGAMINE, Kawasaki-shi (JP)

(21) Appl. No.: 19/154,253

(22) PCT Filed: Jan. 24, 2024
(86) PCT No.: PCT/JP2024/002035

§ 371 Date: Aug. 6, 2025

(30) Foreign Application Priority Data
Feb. 24, 2023 (JP) 2023-027692

(51) Int. Cl. G03F 7/039; C07C 381/12; C07D 333/76; C08F 220/18; C08F 236/20; G03F 7/004

(52) U.S. Cl. CPC G03F 7/039; C07C 381/12; C07D 333/76; C08F 220/1806; C08F 236/20; G03F 7/0045

(57) ABSTRACT

A resist composition that uses a polymer compound, which has a constitutional unit derived from a compound represented by General Formula (a0-m) and generates an acid upon exposure, as a base resin. In Formula (a0-m), W⁰ represents a polymerizable group-containing group; R⁰ represents a hydrocarbon group, an acyl group, an alkoxyalkyl group, or a hydrogen atom; L⁰¹ represents a divalent linking group or a single bond; R^{ar} represents an aromatic ring to which an iodine atom is bonded as a substituent; L⁰² represents a divalent linking group; n0 represents an integer of 1 or more; m represents an integer of 1 or more; and (M^{m+})_{n0/m} represents a counter cation. Improvements in sensitivity when forming a resist pattern and in lithography characteristics such as dimensional uniformity a achieved, and the process margin is widened, when the resist composition is used

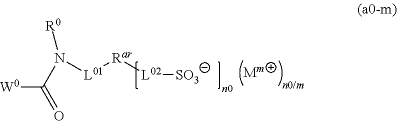

### TECHNICAL FIELD

The present invention relates to a resist composition, a resist pattern formation method, a compound, and a polymer compound.

Priority is claimed on Japanese Patent Application No. 2023-027692, filed Feb. 24, 2023, the content of which is incorporated herein by reference.

### BACKGROUND ART

In recent years, in the manufacture of semiconductor elements and liquid crystal display elements, advances in lithography technologies have led to a rapid progress in the field of pattern fining. These pattern fining techniques typically involve shortening the wavelength (increasing the energy) of the exposure light source.

Resist materials are required to have lithography characteristics such as sensitivity to these exposure light sources and resolution that enables reproduction of patterns with minute dimensions.

As a resist material that satisfies these requirements, a chemically amplified resist composition containing a base material component whose solubility in a developing solution is changed by an action of an acid and an acid generator component that generates an acid upon light exposure has been used in the related art.

In the chemically amplified resist composition, a resin having a plurality of constitutional units is typically used as the base material component in order to improve lithography characteristics and the like. A wide variety of acid generators have been suggested as the acid generator. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an imino sulfonate-based acid generator, and a disulfone-based acid generator.

In addition, in the chemically amplified resist composition, a polymer compound into which a constitutional unit containing an acid generating group that generates an acid upon light exposure is introduced is suggested as the acid generator component (for example, see Patent Document 1). Such a polymer compound has both a function as an acid generator and a function as a base material component.

### CITATION LIST

### Patent Document

[Patent Document 1] Japanese Unexamined Patent Application. First Publication No. 2014-197168

### SUMMARY OF INVENTION

### Technical Problem

With the further progress of the lithography technologies and resist pattern miniaturization, for example, the goal in lithography using extreme ultraviolet (EUV) rays or electron beams (EB) is to form a minute pattern with a size of several tens of nanometers. As the pattern dimension is smaller, both the lithography characteristics such as high sensitivity to an exposure light source and dimensional uniformity are required. In addition, it is also required that the process margin is wide, such as the influence on the pattern size due to the fluctuation of the exposure amount.

However, in the resist composition in the related art as described in Patent Document 1, further improvement is required in terms of these required characteristics.

The present invention has been made in view of the above circumstances, and an object thereof is to provide a resist composition having a wide process margin, in which sensitivity in a case of forming a resist pattern and lithography characteristics such as dimensional uniformity are improved, a resist pattern formation method using the resist composition, a polymer compound useful for the resist composition, and a compound that can be used for synthesis of the polymer compound.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a resin component (A1) whose solubility in a developing solution is changed by the action of the acid, in which the resin component (A1) has a constitutional unit (a0) derived from a compound represented by General Formula (a0-m).

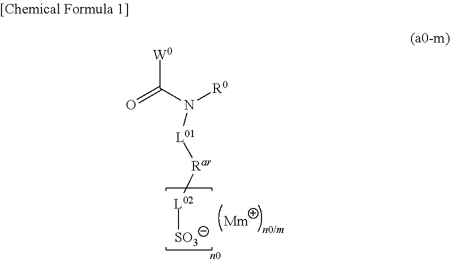

[In the formula, W⁰ represents a polymerizable group-containing group. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, m represents an integer of 1 or more, and (M^{m+})_{n0/m} represents a counter cation.

According to a second aspect of the present invention, there is provided a resist pattern formation method, including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (a0-m).

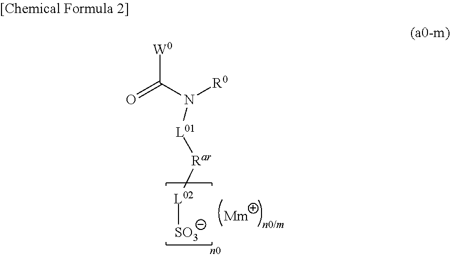

[In the formula, W⁰ represents a polymerizable group-containing group. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, n represents an integer of 1 or more, and (M^{m+})_{n0/m} represents a counter cation.

According to a fourth aspect of the present invention, there is provided a polymer compound which has a constitutional unit derived from the compound according to the third aspect.

### Advantageous Effects of Invention

According to the present invention, it is possible to provide a resist composition in which sensitivity in a case of forming a resist pattern and lithography characteristics such as dimensional uniformity are improved and a process margin is wide, a resist pattern formation method using the resist composition, a polymer compound useful for the resist composition, and a compound that can be used for synthesis of the polymer compound.

### DESCRIPTION OF EMBODIMENTS

In the present specification and the scope of the present claims, the term “aliphatic” is a relative concept used with respect to “aromatic” and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid decomposable group” indicates a group having acid decomposability in which at least a pat of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers typically non-polymers having a molecular weight of 500 or greater and less than 4000 (hereinafter, referred to as “low-molecular-weight compounds”) are used. Hereinafter, the term “resin”. “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{ax}) that substitutes the hydrogen atom bonded to the carbon atom at the α-position is an atom other than the hydrogen atom or a group. Further, itaconic acid diester in which the substituent (R^{ax}) has been substituted with a substituent having an ester bond or α-hydroxyacyl ester in which the substituent (R^{ax}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group; and those obtained by bonding a substituent other than a hydroxyl group to a target compound in which the hydrogen atom at the α-position may be substituted with a substituent. In addition, the α-position denotes the first carbon atom adjacent to the functional group unless otherwise specified.

Examples of the substituent that substitutes a hydrogen atom at the α-position of hydroxystyrene include the same groups as those for R^{ax}, such as an alkyl group and a halogenated alkyl group.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

The resist composition contains a base material component (A) (hereinafter, also referred to as “component (A)”) whose solubility in a developing solution is changed by the action of the acid. In the resist composition of the present embodiment, the component (A) includes a resin component which generates an acid upon light exposure and whose solubility in a developing solution is changed by the action of the acid. This resin component has a constitutional unit (a0) derived from a compound represented by General Formula (a0-m) described later.

In a case where a resist film is formed using the resist composition according to the present embodiment and the formed resist film is subjected to selective exposure, an acid is generated at least from the component (A) at exposed portions of the resist film, and the generated acid acts on the component (A) to change the solubility of the component (A) in a developing solution, whereas the solubility of the component (A) in a developing solution is not changed at unexposed portions of the resist film, and thus a difference in solubility in the developing solution occur between the exposed portions and the unexposed portions of the resist film. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative-tone.

The resist composition of the present embodiment may be a positive-tone resist composition or a negative-tone resist composition. Further, in the formation of a resist pattern, the resist composition according to the present embodiment may be used for an alkali developing process using an alkali developing solution in the developing treatment, or used for a solvent developing process using a developing solution containing an organic solvent (organic developing solution) in the developing treatment.

### <Base Material Component (A)>

In the resist composition according to the present embodiment, the component (A) contains a resin component (A1) (hereinafter, also referred to as “component (A1)”) whose solubility in a developing solution is changed by the action of an acid.

By using the component (A1), it is possible to obtain a good development contrast not only in an alkali developing process but also in a solvent developing process.

As the component (A), at least one of other polymer compounds or low-molecular-weight compounds may be used in combination with the component (A1).

In the resist composition according to the present embodiment, the component (A) may be used alone or in combination of two or more kinds thereof.

### In Regard to Resin Component (A1)

The component (A1) is a resin component which generates an acid upon exposure and whose solubility in a developing solution changes by the action of the acid. The component (A1) has a constitutional unit (a0) derived from a compound represented by General Formula (a0-m).

Further, the component (A1, may have other constitutional units as necessary in addition to the constitutional unit (a0).

<<Constitutional Unit (a0)>>

The constitutional unit (a0) is a constitutional unit derived from a compound represented by General Formula (a0-m). This constitutional unit (a0) generates an acid by exposure in the formation of a resist pattern.

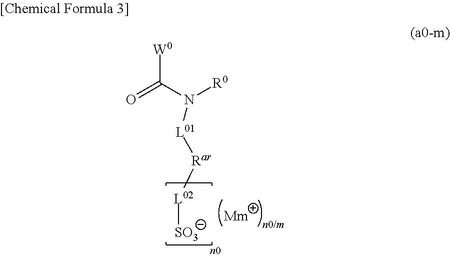

[In the formula, W⁰ represents a polymerizable group-containing group. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, n represents an integer of 1 or more, and (M^{m+})_{n0/m} represents a counter cation.

### [Anion Moiety]

In Formula (a0-m), the term “polymerizable group” in the polymerizable group-containing group as W⁰ is a group that enables polymerization of a compound containing a polymerizable group by radical polymerization or the like and, for example, is a group having multiple bonds between carbon atoms such as an ethylenic double bond.

Examples of the polymerizable group include a vinyl group, an α-methylvinyl group, an allyl group, an acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonyliluorobutylacryloyl group, a vinyl ether group, a fluorine-containing vinyl ether group, an allyl ether group, a fluorine-containing allyl ether group, a styryl group, a vinyinaphthyl group, a fluorine-containing styryl group, a fluorine-containing vinylnaphthyl group, a norbornyl group, a fluorine-containing norbornyl group, a silyl group, and a maleimide group.

The polymerizable group-containing group may be a group formed of only a polymerizable group, or a group formed of a polymerizable group and a group other than the polymerizable group. Examples of the group other than the polymerizable group include a divalent hydrocarbon group which may have a substituent and a divalent linking group having a heteroatom.

Suitable examples of the polymerizable group-containing group include a group represented by Formula: C(R^{X11})(R^{X12})═C(R^{X13})-Ya^{x0}-, in the formula, R^{X11}, R^{X12}, and R^{X13} each independently represent an alkyl group having 1 to 5 carbon atoms, a halogenated alkyl group having 1 to 5 carbon atoms, a halogen atom, or a hydrogen atom, and Ya^{x0} represents a single bond or a divalent linking group. Examples of the divalent linking group as Ya^{x0} include a divalent hydrocarbon group which may have a substituent and a divalent linking group having a heteroatom. Examples of the divalent linking group as Ya^{x0} include an ester bond (—C(═O)—O—), an oxycarbonyl group (—O—C(═O)—), an ether bond (—O—), a linear or branched alkylene group, an arylene group and a combination thereof.

In Formula (a0-m), examples of the hydrocarbon group in R⁰ include a chain-like hydrocarbon group and a cyclic hydrocarbon group.

The chain-like hydrocarbon group in R⁰ may be a saturated aliphatic hydrocarbon group or an unsaturated aliphatic hydrocarbon group. Regarding the saturated aliphatic hydrocarbon group, the linear one (linear alkyl group) preferably has 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group and ethyl group is preferable, and a methyl group is particularly preferable. The branched chain (branched chain alkyl group) preferably has 3 to 10 carbon atoms, and more preferably 3 to 5 carbon atoms. Specific examples thereof include an isopropyl group, an isobutyl group, a ten-butyl group, an isopentyl group, a neopentyl group, a 1,1-diethylpropyl group, and a 2:2-dimethylbutyl group. Examples of the unsaturated aliphatic hydrocarbon group include an alkenyl group and an alkynyl group.

The cyclic hydrocarbon group in R⁰ may be an aliphatic hydrocarbon group, an aromatic hydrocarbon group, a polycyclic group, or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group formed by removing one hydrogen atom from a polycycloalkane is preferable. The number of carbon atoms in the polycycloalkane is preferably 7 to 12, and specifically, adamantane, norborane, isobornane, tricyclodecane, and tetracyclododecane are exemplary examples.

As the aromatic hydrocarbon group, a group in which one or more hydrogen atoms have been removed from an aromatic hydrocarbon ring having 6 to 15 carbon atoms is preferable, a group in which one or more hydrogen atoms have been removed from benzene, naphthalene, anthracene, or phenanthrene is more preferable, a group in which one or more hydrogen atoms have been emmoved from benzene, naphthalene, or anthracene is still more preferable, a group in which one or more hydrogen atoms have been removed from benzene or naphthalene is particularly preferable, and a group in which one or more hydrogen atoms have been removed from benzene is most preferable. Alternatively, examples of the aromatic hydrocarbon group include a group obtained by removing one or more hydrogen atoms from an aromatic compound (for example, biphenyl or fluorene) having two or more aromatic rings; and a group (for example, an arylalkyl group such as a benzyl group, a phenethyl group, a 1-naphthylmethyl group, a 2-naphthylmethyl group, a 1-naphthylethyl group, or a 2-naphthylethyl group) in which one hydrogen atom of a hydrogen atom of an aryl group (an aromatic hydrocarbon ring having 6 to 15 carbon atoms) is substituted with an alkylene group. The number of carbon atoms in the alkylene group bonded to the aryl group is preferably 1 to 4, more preferably 1 or 2, and particularly preferably 1.

The acyl group in R⁰ is preferably an acyl group having 1 to 5 carbon atoms, more preferably an acyl group having 1 to 3 carbon atoms, and particularly preferably an acetyl group.

The alkoxyalkyl group in R⁰ is preferably an alkoxyalkyl group having 2 to 10 carbon atoms, more preferably an alkoxyalkyl group having 2 to 6 carbon atoms, and particularly preferably a methoxymethyl group.

R⁰ is preferably a linear alkyl group or a hydrogen atom, and among these, a hydrogen atom is more preferable.

In Formula (a0-m), L⁰¹ represents a divalent linking group or a single bond.

Examples of the divalent linking group in L⁰¹ include a divalent hydrocarbon group which may have a substituent and a divalent linking group having a heteroatom.

Divalent Hydrocarbon Group which May have Substituent:

The divalent hydrocarbon group which may have a substituent as L⁰¹ may be an aliphatic hydrocarbon group or may be an aromatic hydrocarbon group.

### Aliphatic Hydrocarbon Group

The aliphatic hydrocarbon group indicates a hydrocarbon group that has no aromaticity. The aliphatic hydrocarbon group may be saturated or may be unsaturated (for example, an alkenylene group, an alkynylene group, or the like).

Examples of the aliphatic hydrocarbon group include a chain-like aliphatic hydrocarbon group, and an aliphatic hydrocarbon group containing a ring in the structure thereof. The chain-like aliphatic hydrocarbon group may be a linear aliphatic hydrocarbon group or may be a branched aliphatic hydrocarbon group.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₂CH₂—, and —C(CH₂CH₃)₂—CH₂—; alkyltrimethylene groups such as —CH—(CH₃)CH₂CH₂, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may have or may not have a substituent.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof in group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane is preferably a monocycloalkane having 3 to 6 carbon atoms, and specific examples thereof include cyclopropane, cyclopentane, and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxy group, and a carbonyl group.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxy group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom

Examples of the divalent linking group having a heteroatom as L⁰¹ include —O—, —C(═O)—O—, —O—C(═O)—, —C(═O)—, —O—C(═O)—O—, —C(═O)—NH—, —C(═O)—NH—C(═O)—, —NH—, —NH—C(═NH)— (H may be substituted with a substituent such as a hydrocarbon group, an acyl group, or an alkoxyalkyl group), —S—, —S(═O)₂—, —S(═O)₂—O—, and a group represented by General Formula —Y²¹-Y²²—, —Y²¹—O—, —Y²¹—C(═O)—, —C(═U)—(—Y²¹—, —[Y²¹—C(═O)—O]ₘ—Y22-, —Y²¹—O—C(═O)—Y²²—, or —Y²¹—S(═O)₂—O—Y²²— [in the formulae. Y²¹ and Y²² each independently represent a divalent hydrocarbon group which may have a substituent, O represents an oxygen atom, and m″ represents an integer of 1 to 3].

In a case where the divalent linking group having a heteroatom is —C(═O)—NH—, —C(═O)—NH—C(═O)—, —NH—, or —NH—C(═NH)—, H of the divalent linking group may be substituted with a substituent such as a hydrocarbon group, an acyl group, or an alkoxyalkyl group. The number of carbon atoms in the substituent is preferably in a range of 1 to 10, more preferably in a range of 1 to 8, and particularly preferably in a range of 1 to 5.

In General Formula —Y²¹—O—Y²²—, —Y²²—O—, —Y²¹—C(═O)—O—, —C(═O)—O—Y²¹—, —[Y²¹—C(═O)—O]ₘ—Y²²—, —Y²²— —C(═O)—Y²²—, or —Y²¹—S(═O)₂—O—Y²²—, Y²¹ and Y²² each independently represent a divalent hydrocarbon group which may have a substituent. Examples of the divalent hydrocarbon group include the same groups as described above.

Aa Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable. The alkyl group in the alkylmethylene group is preferably a linear alkyl group having 1 to 5 carbon atoms, more preferably a linear alkyl group having 1 to 3 carbon atoms, and most preferably a methyl group.

As L⁰¹, an ester bond (—C(═O)—O—), an oxycarbonyl group (—O—C(═O)—), —C(═O)—, an ether bond (—O—), a linear or branched alkylene group, or a combination thereof is preferable; and a combination of a chain-like alkylene group and an ester bond, or —C(═O)— is more preferable.

L⁰¹ is preferably a single bond.

In Formula (a0-m), the aromatic ring in R^{ar} is not particularly limited as long as it is a cyclic conjugated system having 4n+2 π electrons, and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring in R^{ar} include aromatic hydrocarbon rings such as benzene, naphthalene, anthracene, and phenanthrene, and benzene or naphthalene is preferable and benzene is more preferable.

The number of iodine atoms bonded to the aromatic ring in R^{ar} is appropriately determined depending on the structure of the aromatic ring, and from the viewpoint of sensitivity and lithography characteristics, it is preferably 2 or more.

For example, in a case where the aromatic ring in R^{ar} is benzene, the number of iodine atoms bonded to the benzene is preferably 1 to 3, more preferably 2 or 3, and still more preferably 2.

Examples of the substituent other than the iodine atom, which may be bonded to the aromatic ring in R^{ar}, include an alkyl group, an alkoxy group, a halogen atom other than the iodine atom, a halogenated alkyl group, and a hydroxy group. The number of carbon atoms in each of the alkyl group, the alkoxy group, and the halogenated alkyl group is preferably 1 to 5. Examples of the halogen atom other than the iodine atom preferably include a fluorine atom. Examples of the halogenated alkyl group preferably include a fluorinated alkyl group.

In Formula (a0-m), examples of the divalent linking group in L⁰² include a divalent linking group having a heteroatom. Examples of the divalent linking group having a heteroatom in L⁰² include a divalent linking group obtained by combining a linking group having an oxygen atom and a divalent hydrocarbon group which may have a substituent.

Examples of the linking group having an oxygen atom include —O—, —C(═O)—O—, —O—C(═O)—, —C(═O)—, —O—C(═O)—O—, —C(═O)—NH—, —C(—=)—NH—C(═O)—, —S(═O)₂, and —S(═O)₂—O—. Examples of the divalent hydrocarbon group which may have a substituent include the same ones a, those of the “divalent hydrocarbon group which may have a substituent in L⁰¹” described above.

Examples of the preferred divalent linking group in L⁰² include a linking group represented by General Formula (L⁰²-1), in the formula, * represents a side bonded to R^{ar} in Formula (a0-m). ** represents a side bonded to a sulfur atom (S) in SO₃⁻ in Formula (a0-m).

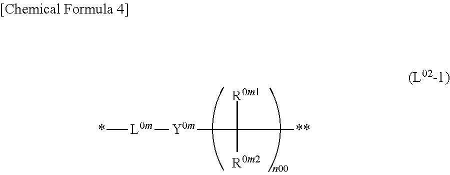

[In the formula, L^{0m}, is a divalent linking group. Y^{0m} represents a divalent linking group which may have a heteroatom or a single bond. R^{0m1} and R^{0m2} each independently represent a fluorine atom, a fluorinated alkyl group, or a hydrogen atom, n00 represents an integer of 0 to 5].

In Formula (L⁰²-1), examples of the divalent linking group as L^{0m} include a non-hydrocarbon-based oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and a combination of the non-hydrocarbon-based oxygen atom-containing linking group and an alkylene group. Further, a sulfonyl group (—SO₂—) may be further linked to the combination.

Examples of the divalent linking group in L^{0m} include linking groups each represented by General Formulae (L-al-1) to (L-al-9).

Further, in General Formulae (L-al-1) to (L-al-9). V′¹⁰¹ in General Formulae (L-al-1) to (L-al-9) is bonded to R^{ar} in Formula (a0-m).

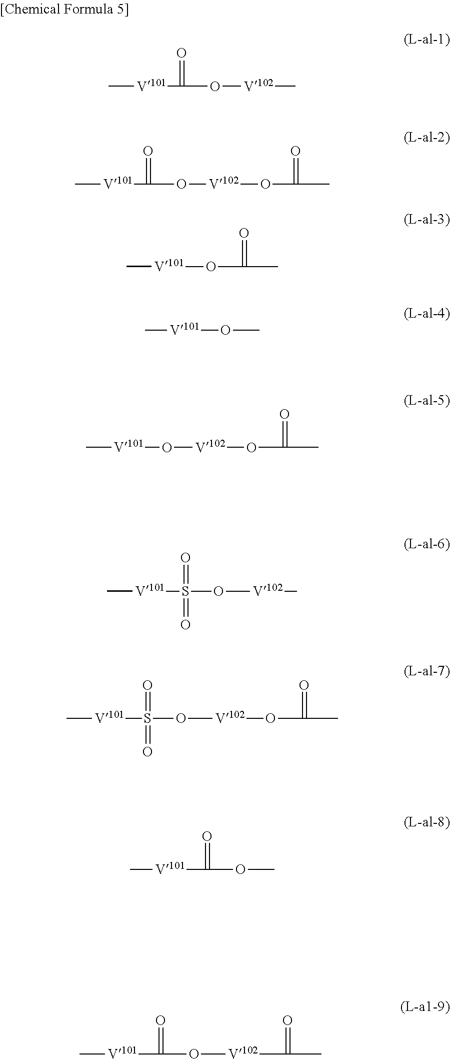

[in the formula, V′¹⁰¹ represents an alkylene group having 1 to 5 carbon atoms or a single bond. V′¹⁰² represents a divalent saturated hydrocarbon group having 1 to 30 carbon atoms].

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable. Alternatively, the divalent saturated hydrocarbon group in V′¹⁰² may be a divalent aliphatic cyclic group. This divalent aliphatic cyclic group may be a polycyclic group or a monocyclic group. The aliphatic cyclic group which is a monocyclic group is preferably a group in which two hydrogen atoms have been removed from a monocycloalkane. The monocycloalkane has preferably 3 to 6 carbon atoms, and specific examples thereof include cyclopentane and cyclohexane. As the aliphatic cyclic group which is a polycyclic group, a group formed by removing two hydrogen atoms from a polycycloalkane is preferable. The number of carbon atoms in the polycycloalkane is preferably 7 to 12, and specifically, adamantane, norbornane, isobornane, tricyclodecane, and tetracyclododecane are exemplary examples.

The alkylene group as V′¹⁰¹ and V′¹⁰² may be a linear alkylene group or a branched chain-like alkylene group, and a linear alkylene group is preferable.

Further, a pan of methylene groups in the alkylene group as V′¹⁰¹ and V′¹⁰² may be substituted with a divalent aliphatic cyclic group having 5 to 10 carbon atoms. The aliphatic cyclic group is preferably a divalent group obtained by removing a hydrogen atom from an aliphatic hydrocarbon ring (a monocyclic aliphatic hydrocarbon ring or a polycyclic aliphatic hydrocarbon ring), and examples thereof include a cyclohexylene group, a 1,5-adamantylene group, and a 2,6-adamantylene group.

As L^{0m}, a divalent linking group including an ester bond or a divalent linking group including an ether bond is preferable, and the divalent linking groups represented by each of Formulae (L-al-1) to (L-al-5), (L-al-8), and (L-al-9) are more preferable.

In Formula (L⁰²-1), Y^{0m} is a divalent linking group which may have a heteroatom or a single bond.

The divalent linking group as Y^{0m} is not particularly limited, and suitable examples thereof include a divalent hydrocarbon group which may have a substituent, and a divalent linking group having a heteroatom. In Y^{0m}, the divalent hydrocarbon group which may have a substituent and the divalent linking group which has a heteroatom are the same as the divalent hydrocarbon group which may have a substituent and the divalent linking group which has a heteroatom exemplified as the divalent linking group in L⁰¹ in Formula (a0-m).

Among the above, as Y^{0m}, a linear or branched alkylene group, an aromatic hydrocarbon group which may have a substituent, or a single bond is preferable, and a linear alkylene group, a phenylene group having a fluorine atom as a substituent, or a single bond is more preferable.

In Formula (L⁰²-1), the fluorinated alkyl group in each of R^{0m1} and R^{0m2} is preferably a linear or branched fluorinated alkyl group having 1 to 5 carbon atoms, and more preferably a trifluoromethyl group.

In Formula (a0-m), it is preferable that at least one of R^{0m1} and Ra^{0m2} bonded to the carbon atom adjacent to SO₃⁻ represents a fluorine atom from the viewpoint of acid strength.

In Formula (L⁰²-1), n00 is an integer of 0 to 5, preferably an integer of 1 to 4, and more preferably 1, 2, or 3.

In Formula (a0-m), n0 represents an integer of 1 or more as long as the atomic valence is allowed, and is appropriately determined according to the structure of the aromatic ring in R^{ae} and for example, in a case where the aromatic ring in R^{ar} is benzene, it is preferably 1 or 2, and more preferably 1.

Preferred examples of the constitutional unit (a0) include a constitutional unit represented by General Formula (a0-u1), a constitutional unit represented by General Formula (a0-u2), and a constitutional unit represented by General Formula (a0-u3), which will be described later.

Regarding Constitutional Unit Represented by General Formula (a0-u1)

Examples of the preferred constitutional unit (a0) include a constitutional unit represented by General Formula (a0-u1).

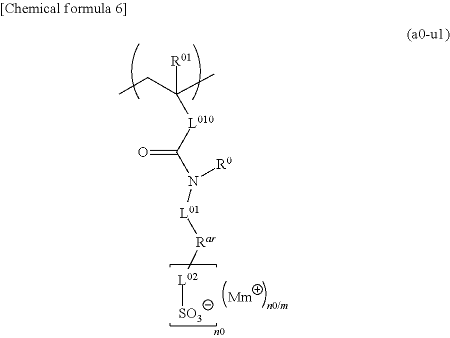

[In the formula. R⁰¹ represents an alkyl group having 1 to 5 carbon atoms, a halogenated alkyl group having 1 to 5 carbon atoms, a halogen atom, or a hydrogen atom. L⁰¹⁰ represents a divalent linking group or a single bond. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, m represents an integer of 1 or more, and (M^{m+})_{n0/m} represents a counter cation.

In Formula (a0-u1), as the alkyl group having 1 to 5 carbon atoms as R⁰¹, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. A fluorine atom is particularly preferable as the halogen atom in the halogenated alkyl group. Examples of the halogen atom as R⁰¹ include a fluorine atom, a chlorine atom, a bromine atom, and an iodine atom.

R⁰¹ represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

Examples of the divalent linking group in L⁰¹¹ in Formula (a0-u1) include a divalent hydrocarbon group which may have a substituent, a divalent linking group which includes a heteroatom, and the like, and examples thereof include the same ones as the divalent hydrocarbon group which may have a substituent and the divalent linking group which includes a heteroatom, which are exemplified in the description of the divalent linking group in L⁰¹ in Formula (a0-m) described above.

L⁰¹⁰ may be a single bond.

Alternatively, L⁰¹⁰ may be a divalent linking group, and among these, an ester bond (—C(═O)—O—), an oxycarbonyl group (—O—C(═O)—), an ether bond (—O—), a linear or branched alkylene group, an arylene group, or a combination thereof is preferable; and a combination of an ester bond and a chain-like alkylene group or an arylene group is particularly preferable.

In Formula (a0-u1). R⁰, L⁰¹, R^{ar}, L⁰², and n0 are the same as R⁰, L⁰¹, R^{ar}, L⁰², and n0 in Formula (a0-m).

Hereinafter, preferred specific examples of the anionic moiety of the constitutional unit represented by General Formula (a0-u1) will be shown. In the formulae shown below, R^{α} represents a hydrogen atom, a methyl group, or a trifluoromethyl group.

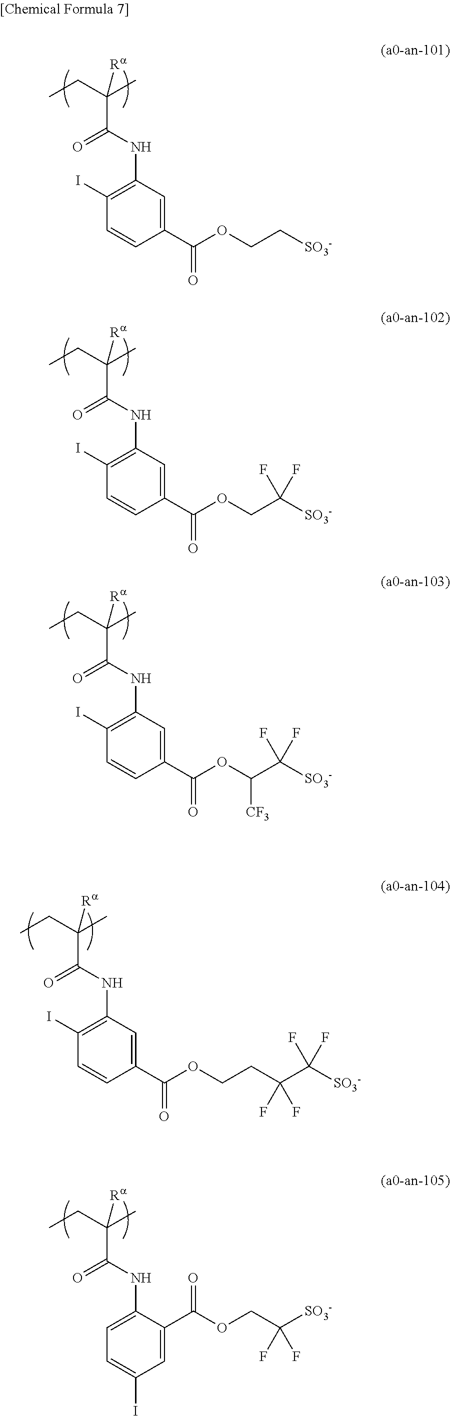

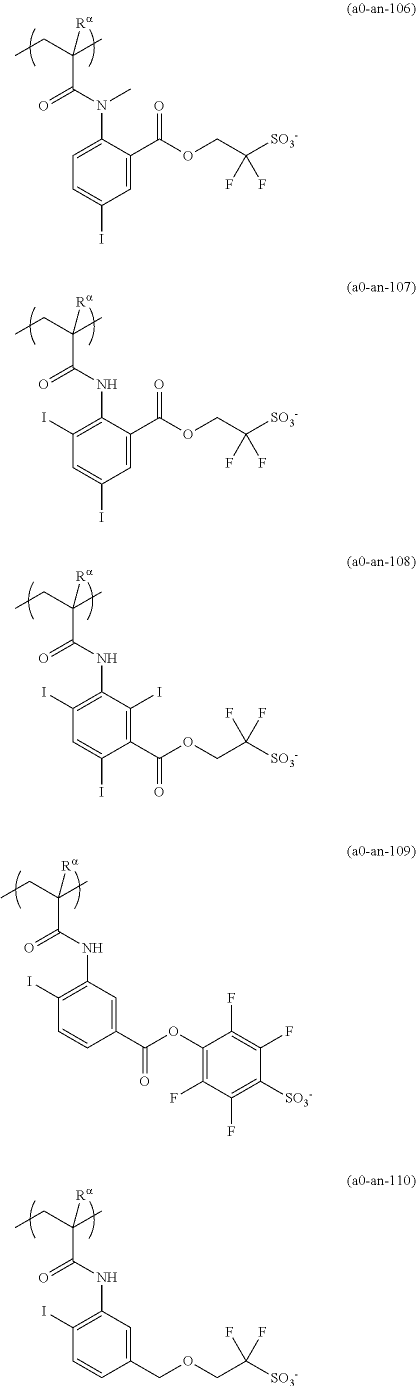

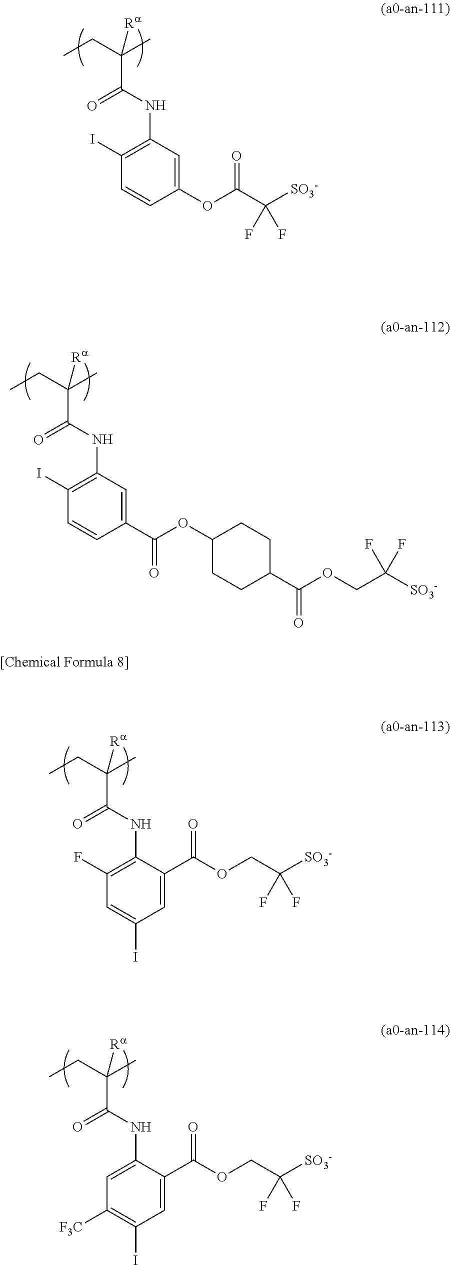

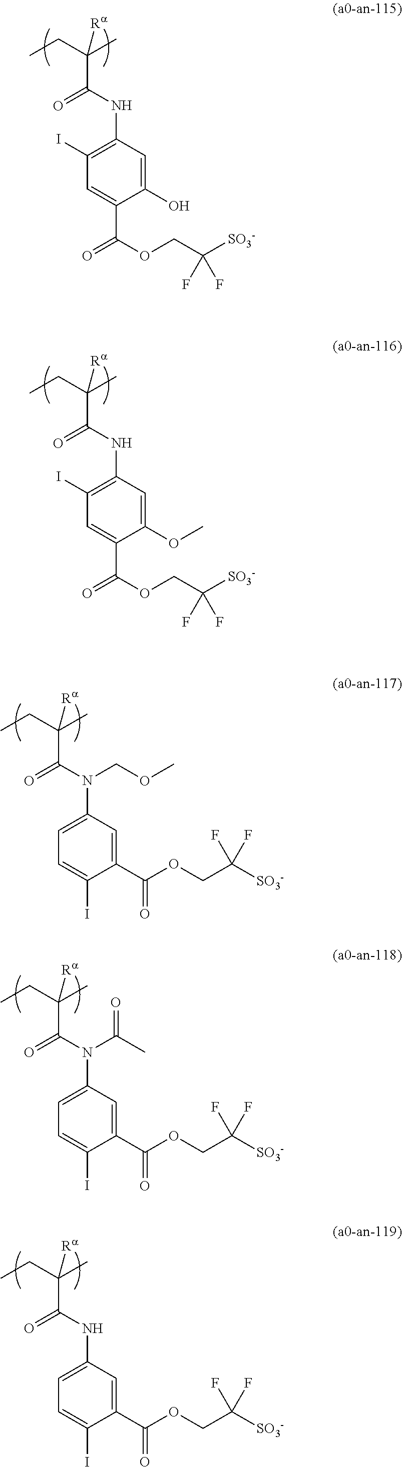

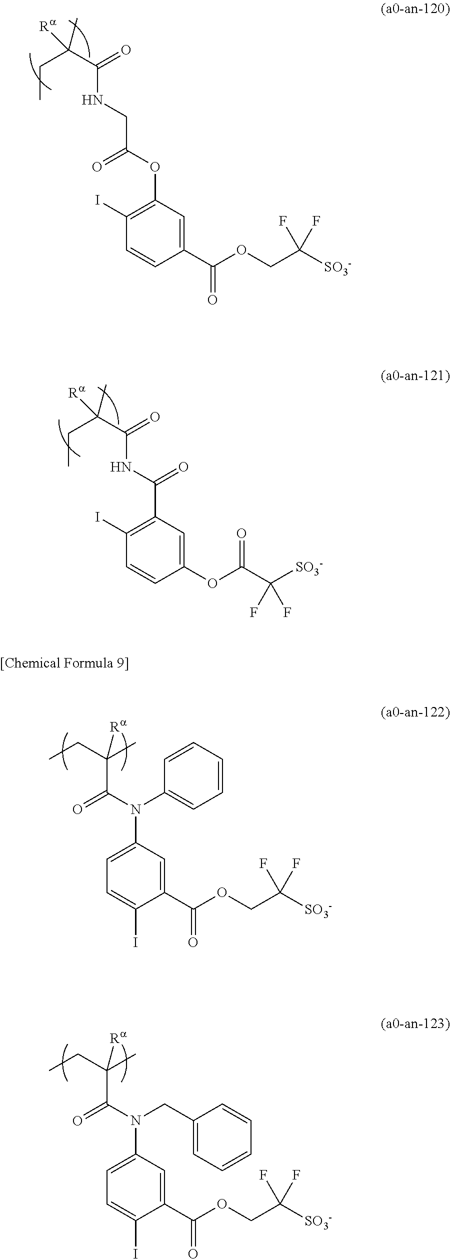

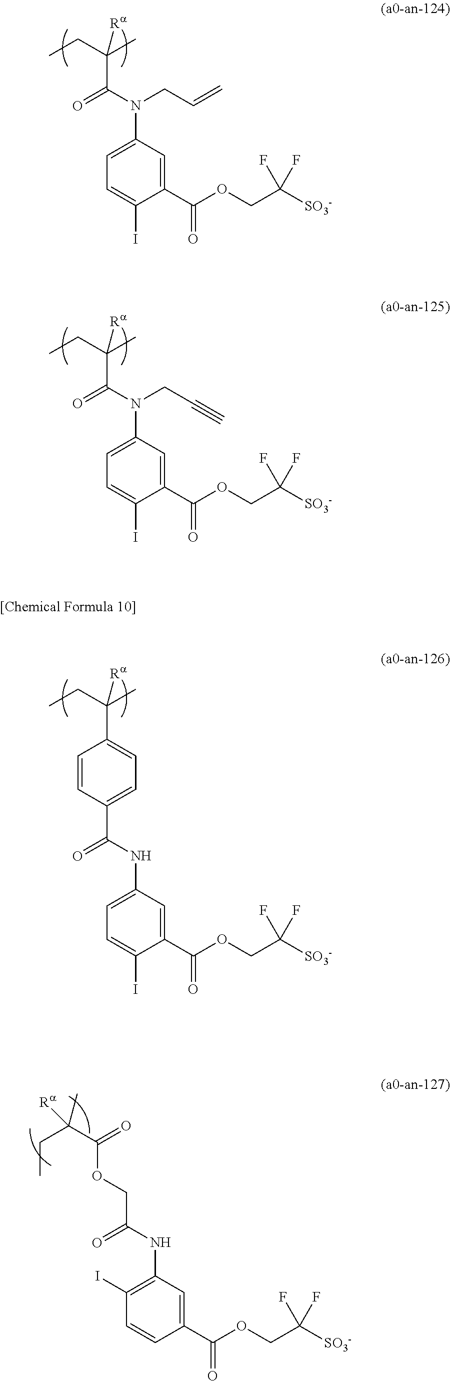

Aa the anionic moiety of the constitutional unit represented by General Formula (a0-u1), at least one kind selected from the group consisting of anions represented by each of Chemical Formulae (a0-an-101) to (a0-an-127) is preferable, and at least one kind selected front the group consisting of anions represented by each of Chemical Formulae (a0-an-101) to (a0-an-116), (a0-an-119), and (a0-an-126) is more preferable.

Regarding Constitutional Unit Represented by General Formula (a0-u2)

Examples of the preferred constitutional unit (a0) include a constitutional unit represented by General Formula (a0-u2).

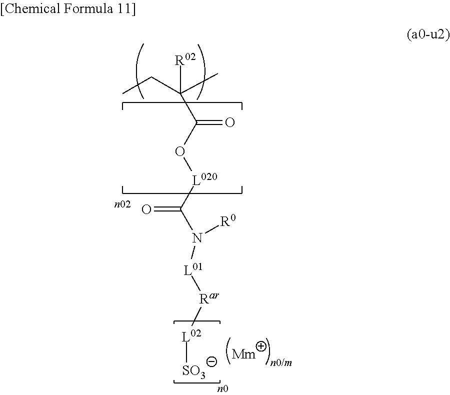

In the formula, R⁰² represents an alkyl group having 1 to 5 carbon atoms, a halogenated alkyl group having 1 to 5 carbon atoms, a halogen atom, or a hydrogen atom, L⁰²⁰ represents a divalent linking group, n02 represents an integer of 0 to 2. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring, L⁰² represents a divalent linking group, n0 represents an integer of 1 or more as long as an atomic valence allows, m represents an integer of 1 or more, and (M^{m+})_{n0/m} represents a counter cation.

In Formula (a0-u2), the alkyl group having 1 to 5 carbon atoms, the halogenated alkyl group having 1 to 5 carbon atoms, and the halogen atom in R⁰² are each the same as the alkyl group having 1 to 5 carbon atoms, the halogenated alkyl group having 1 to 5 carbon atoms, and the halogen atom in R⁰¹ of Formula (a0-u1).

R⁰² represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a0-u2), examples of the divalent hydrocarbon group which may have a substituent as the divalent linking group in L⁰²⁰ include the divalent hydrocarbon group which may have a substituent, which is exemplified in the description of the divalent linking group in L⁰¹ of Formula (a0-m).

As the divalent linking group in L⁰²⁰, an aliphatic hydrocarbon group is preferable, a linear or branched alkylene group is more preferable, a linear aliphatic hydrocarbon group is still more preferable, and a linear alkylene group having 1 to 10 carbon atoms is particularly preferable.

In Formula (a0-u2), n02 is an integer of 0 to 2, preferably 0 or 1 and more preferably 0.

In Formula (a0-u2), R⁰, L⁰¹, R^{ar}, L⁰², and n0 are the same as R⁰, L⁰¹, R^{ar}, L⁰², and n0 in Formula (a0-m).

Preferred specific examples of the anionic moiety of the constitutional unit represented by Formula (a0-u2) include anions represented by each of the above-described Formulae (a0-an-101) to (a0-an-125) and (a0-an-127). Among these, at least one kind of anion selected from the group consisting of anions represented by each of the above-described Chemical Formulae (a0-an-101) to (a0-an-116) and (a0-an-119) is preferable, and at least one kind of anion selected from the group consisting of anions represented by each of the above-described Chemical Formulae (a0-an-107) and (a0-an-108) is more preferable.

Regarding Constitutional Unit Represented by General Formula (a0-u3)

Examples of the preferred constitutional unit (a0) include a constitutional unit represented by General Formula (a0-u3).

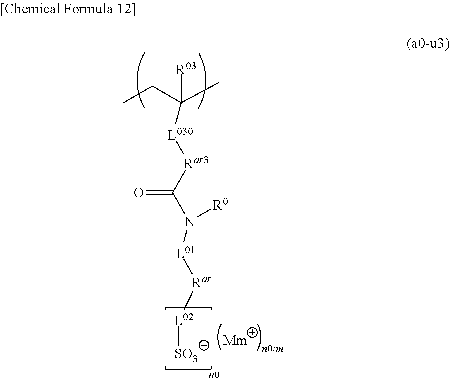

[in the formula, R⁰³ represents an alkyl group having 1 to 5 carbon atom, a halogenated alkyl group having 1 to 5 carbon atoms, a halogen atom, or a hydrogen atom. L⁰³⁰ represents a divalent linking group or a single bond. R^{ar3} represents an aromatic ring, which may have a substituent. R⁶ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, in represents an integer of 1 or more, and (M^{m+})_{n0/m} represents a counter cation.

In Formula (a0-u3), the alkyl group having 1 to 5 carbon atoms, the halogenated alkyl group having 1 to 5 carbon atoms, and the halogen atom in R⁰³ are each the same as the alkyl group having 1 to 5 carbon atoms, the halogenated alkyl group having 1 to 5 carbon atoms, and the halogen atom in R⁰¹ of Formula (a0-u1).

R⁰³ represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a0-u3), examples of the divalent linking group in L⁰³⁰ include a divalent hydrocarbon group which may have a substituent and a divalent linking group which includes a heteroatom, and the same ones as the divalent hydrocarbon group which may have a substituent and the divalent linking group which includes a heteroatom, which are exemplified in the description of the divalent linking group in L⁰¹ in Formula (a0-m), can be mentioned.

L⁰³⁰ may be a single bond.

Alternatively. L⁰³⁰ may be a divalent linking group, and among these, an ester bond (—C(═O)—O—), an oxycarbonyl group (—O—C(═O)—), an ether bond (—O—), a linear or branched alkylene group, or a combination thereof is preferable.

L⁰³⁰ is preferably a single bond.

In Formula (a0-u3), the description for the aromatic ring in R^{ar3} is the same as the description for the aromatic ring in R^{ar} in Formula (a0-m), and examples thereof include an aromatic hydrocarbon ring such as benzene, naphthalene, anthracene, and phenanthrene, where benzene or naphthalene is preferable and benzene is more preferable.

Examples of the substituent which may be contained in the aromatic ring in R^{ar3} include an alkyl group, an alkoxy group, a halogen atom, a halogenated alkyl group, and a hydroxy group. The number of carbon atoms in each of the alkyl group, the alkoxy group, and the halogenated alkyl group is preferably 1 to 5.

In Formula (a0-u3). R⁰, L⁰¹, R^{ar}, L⁰², and n0 are the same as R⁰, L⁰¹, R^{ar}, L⁰², and n0 in Formula (a0-m).

Preferred specific examples of the anionic moiety of the constitutional unit represented by Formula (a0-u3) include the anion represented by the above-described Formula (a0-an-126).

As the anionic moiety of the constitutional unit (a0), at least one kind selected from the group consisting of anions represented by each of Chemical Formulae (a0-an-101) to (a0-an-127) is preferable, at least one kind selected from the group consisting of anions represented by each of Chemical Formulae (a0-an-101) to (a0-an-116), (a0-an-119), and (a0-an-126) is more preferable, and at least one kind selected from the group consisting of anions represented by each of Chemical Formulae (a0-an-107) to (a0-an-108) and (a0-an-126) is still more preferable.

### [Cation Moiety]

In Formula (a0-m), nm represents an integer of 1 or more, and (M^{m+})_{n0/m} represents a counter cation, n0 represents an integer of 1 or more as long as an atomic valence allows.

As the counter cation represented by (M^{m+})_{n0/m}, an onium cation is preferable, and among the examples, a sulfonium cation or an iodonium cation is more preferable.

Preferred examples of the cation moiety (M^{m+})_{n0/m} include onium cations each represented by General Formulae (ca-1) to (ca-3).

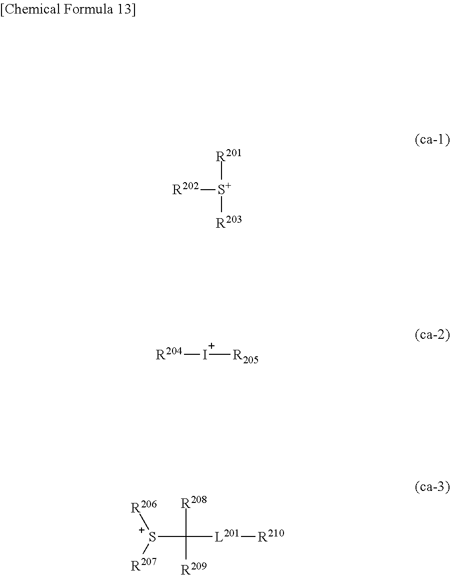

[In the formula, R²⁰¹ to R²⁰⁷ each independently represent an aryl group which may have a substituent, an alkyl group which may have a substituent, or an alkenyl group which may have a substituent. R²⁰¹ to R²⁰³, and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—].

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent which may be contained in R²⁰¹ to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

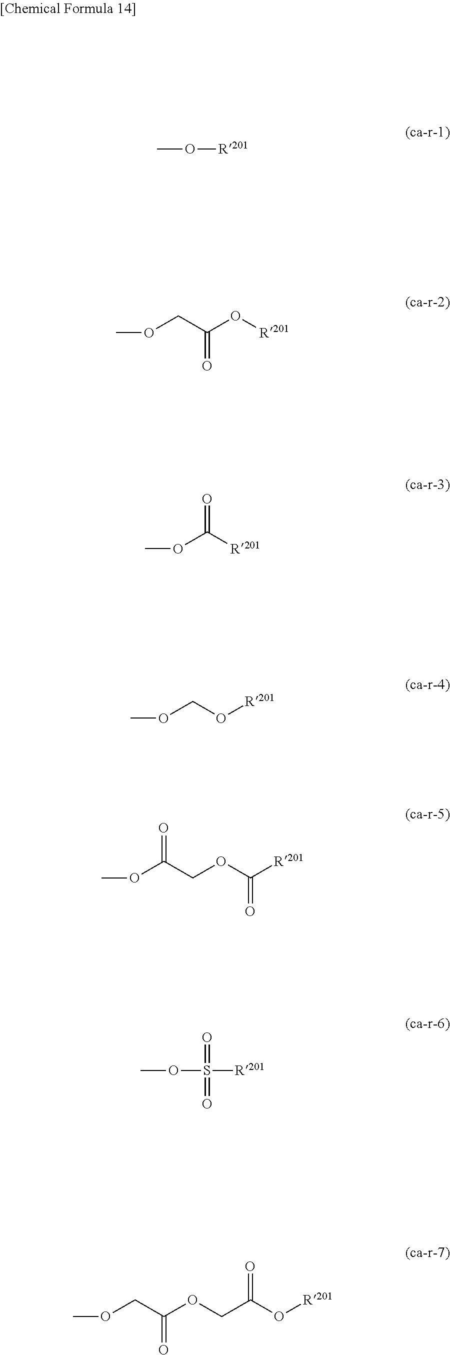

[In the formulae, R′²⁰¹'s each independently represents a hydrogen atom, a cyclic group which may have a substituent, a chain-like alkyl group which may have a substituent, or a chain-like alkenyl group which may have a substituent].

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring-based polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃—), —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16).

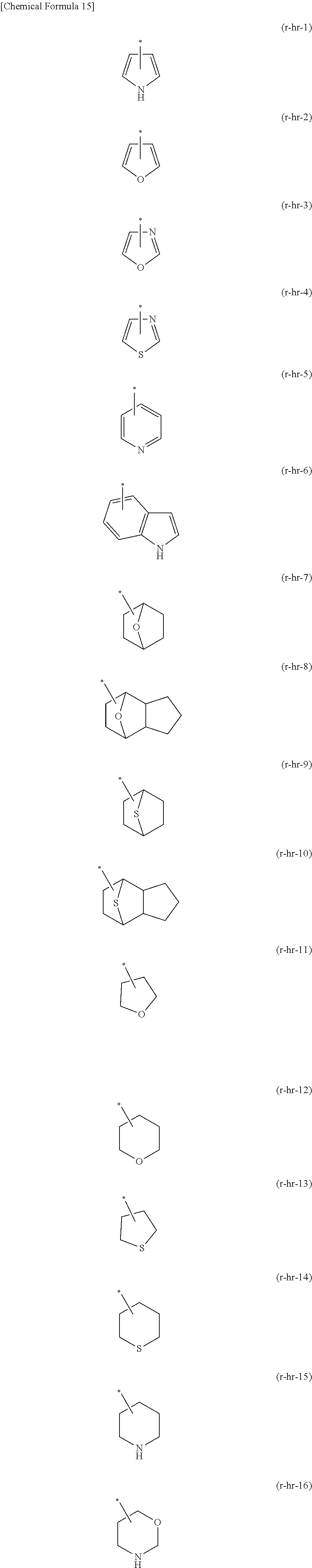

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such a, a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-like alkyl group which may have substituent:

The chain-like alkyl group as R′²⁰¹ may be linear or branched.

The linear alkyl group has preferably 1 to 20 carbon atoms, more preferably 1 to carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include the same groups as those for the acid dissociable group represented by Formula (a1-r-2) described as the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁶ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group. R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms, and a phenyl group or a naphthyl group is preferable.

As the alkyl group represented by R²¹⁰, a chain-like or cyclic alkyl group having 1 to 30 carbon atoms is preferable.

The alkenyl group as R²¹⁰ preferably has 2 to 10 carbon atoms.

The —SO₂-containing cyclic group as R²¹⁰ is not particularly limited, and any —SO₂-containing cyclic group can be used. Specific examples thereof include groups each represented by General Formulae (b5-r-1) to (b5-r-4), where “—SO₂-containing polycyclic group” is preferable, and a group represented by General Formula (b5-r-1) is more preferable.

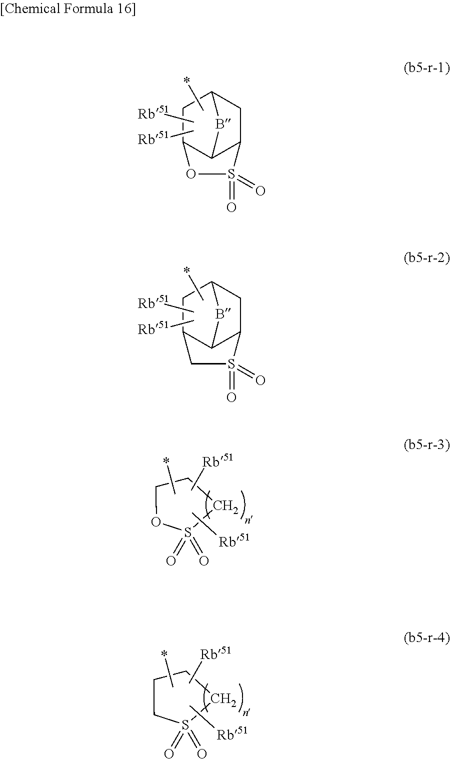

[In the formulae, each Rb′⁵¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an oxygen atom, a sulfur atom, or an alkylene group having 1 to 5 carbon atoms, which may contain an oxygen atom or a sulfur atom; and n′ represents an integer of 0 to 2. ″ represents a bonding site].

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —(OC(═O)R″, a hydroxyalkyl group, or a cyano group, and among the examples, it is preferable that Rb′⁵¹'s each independently represent a hydrogen atom or a cyano group.

Specific examples of the groups each represented by General Formulae (b5-r-1) to (b5-r-4) are shown below. In the formulae shown below. “Ac” represents an acetyl group.

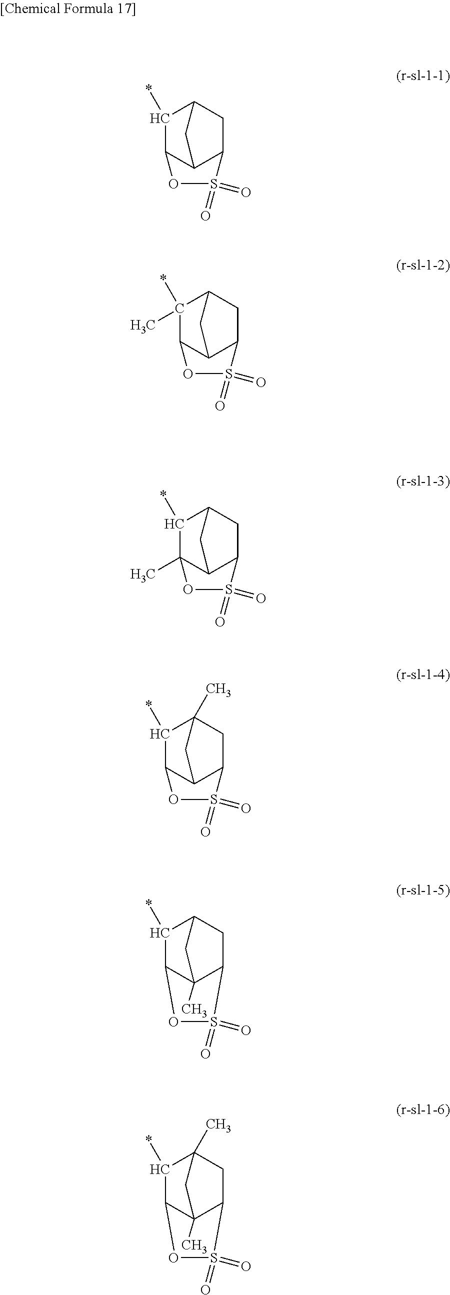

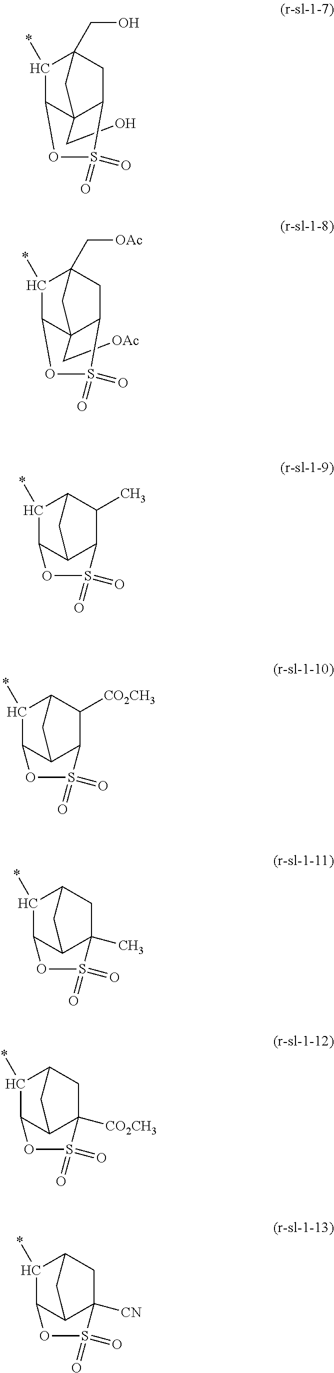

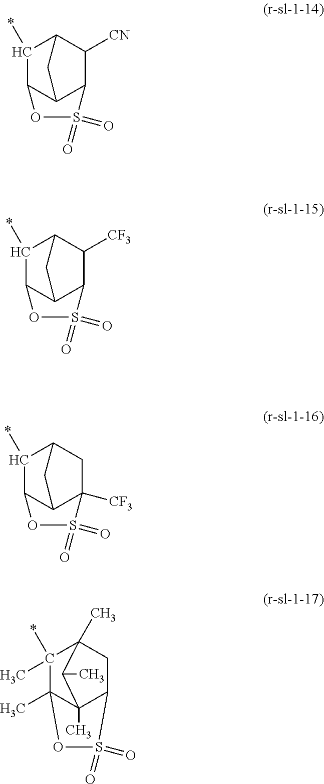

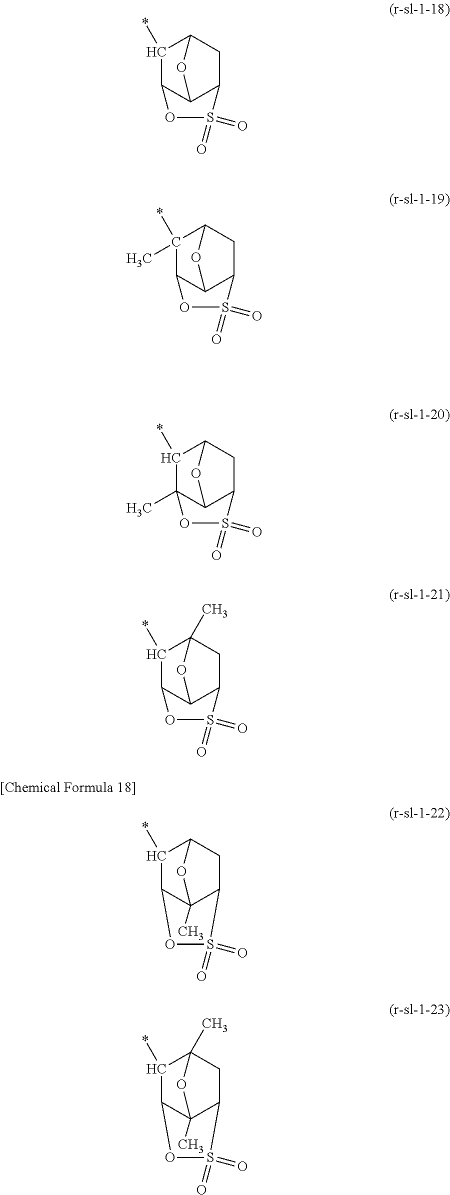

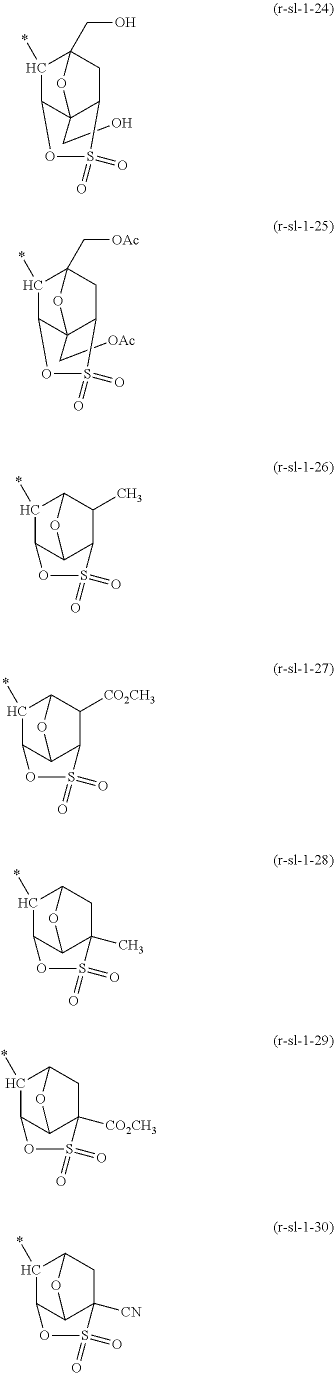

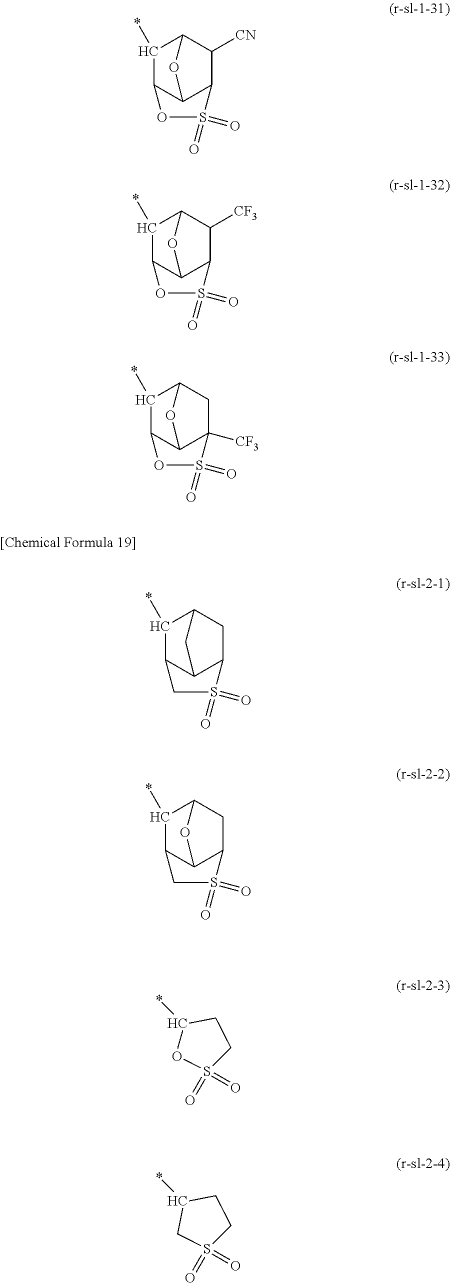

Specific examples of the suitable cation represented by Formula (ca-1) include cations each represented by the following chemical formulae.

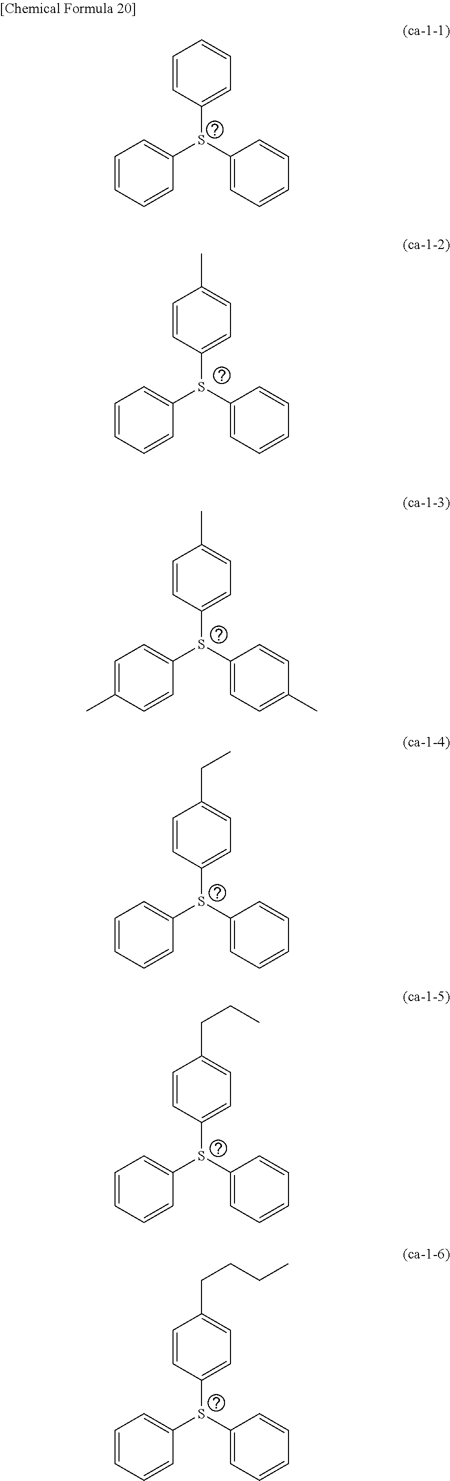

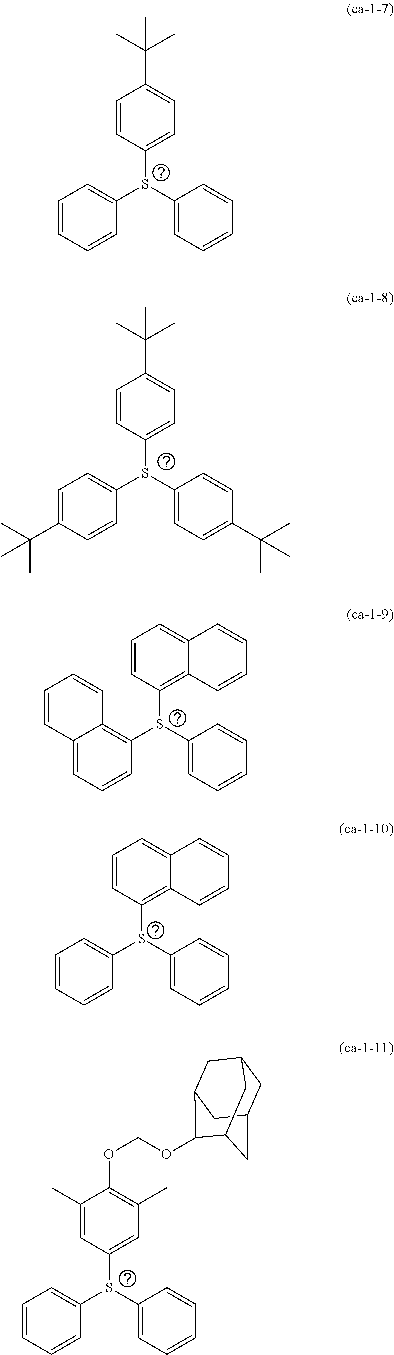

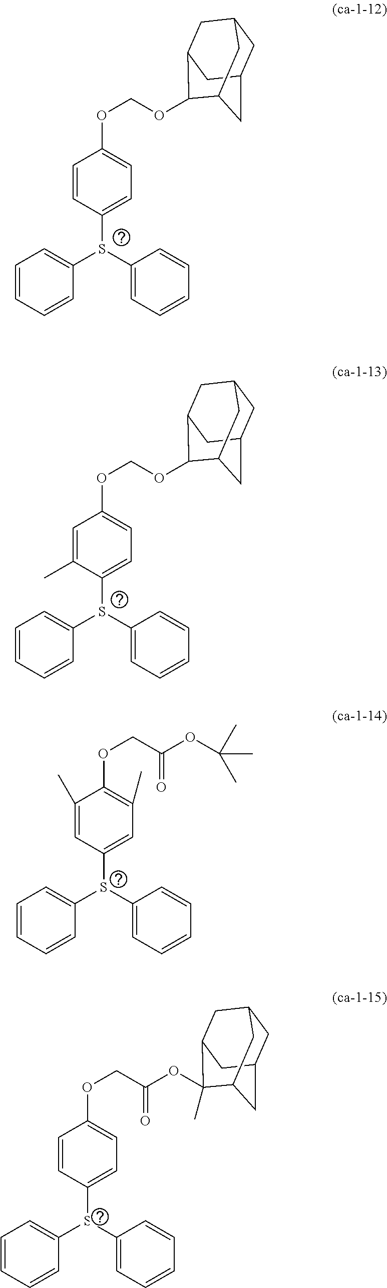

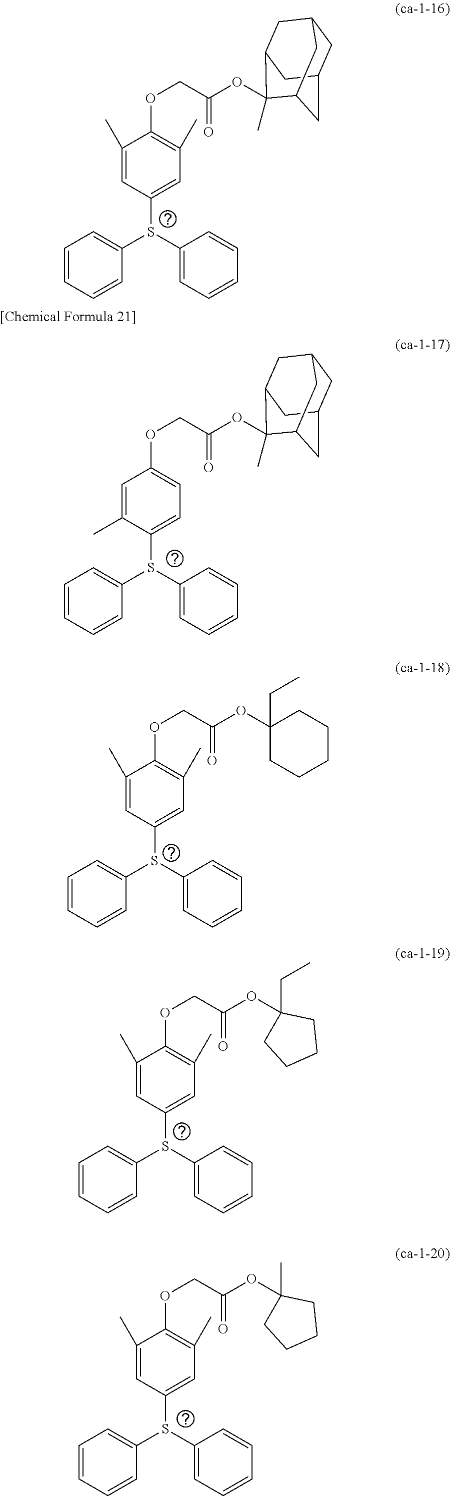

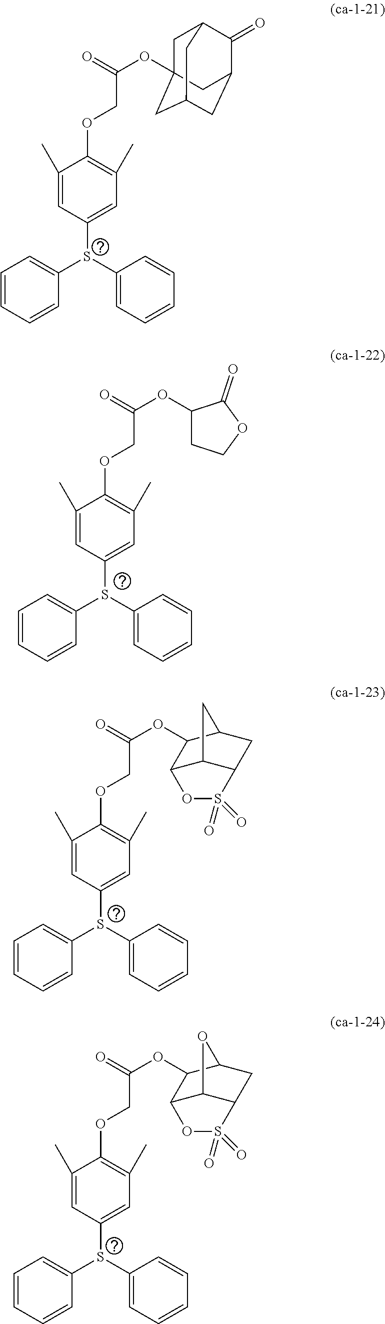

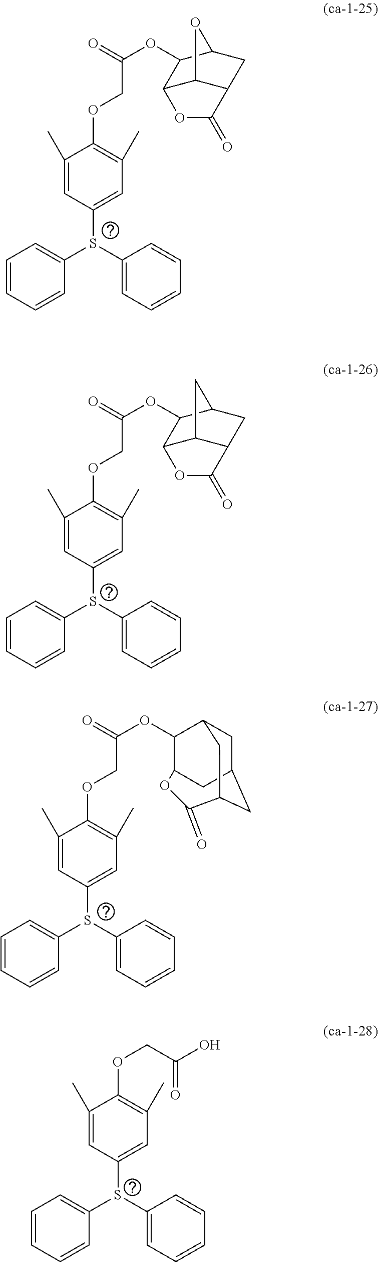

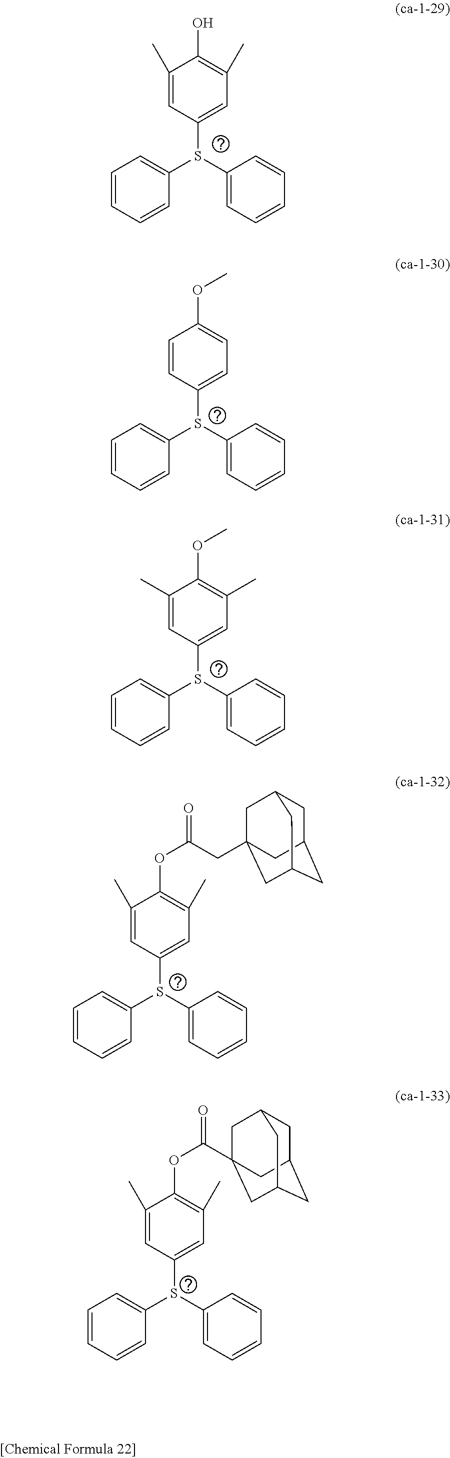

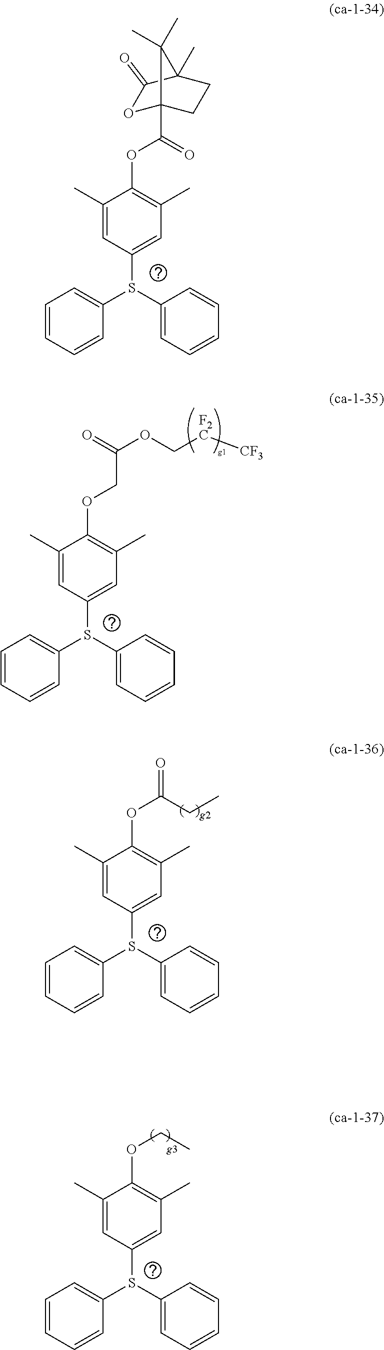

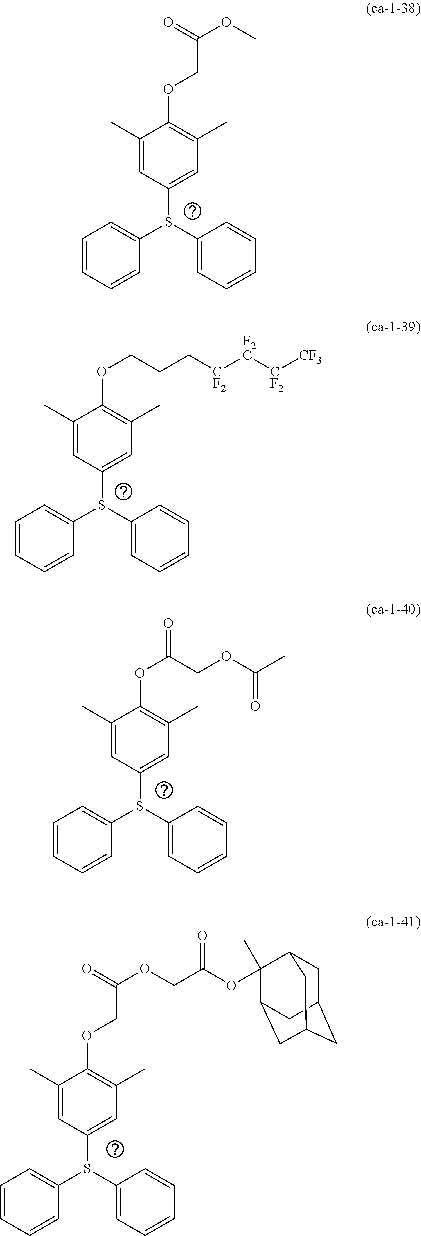

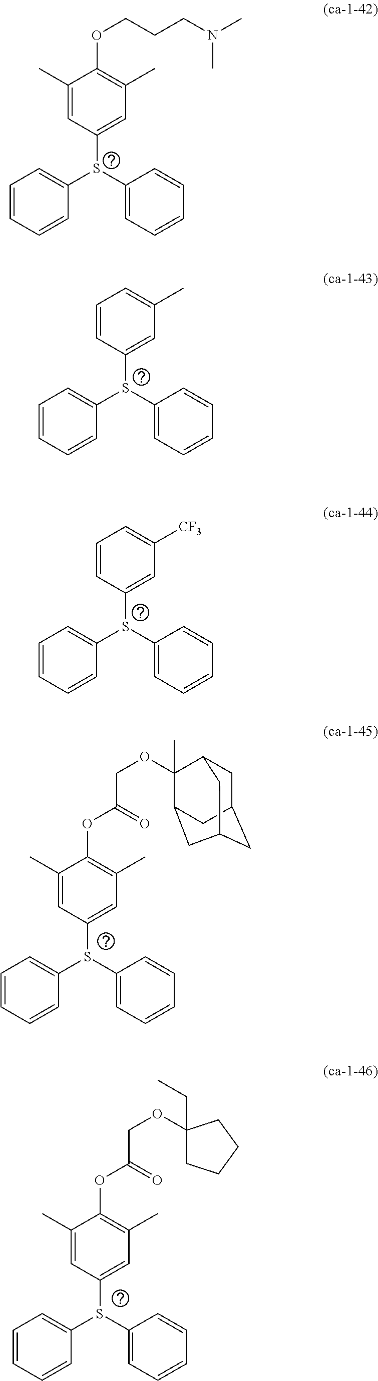

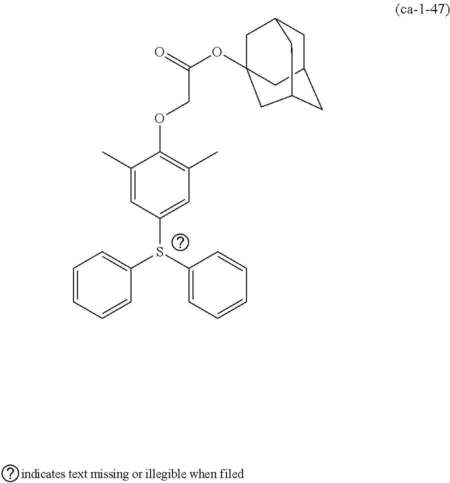

[In the formulae, g1, g2, and g3 represent a repeating number, g1 represents an integer of 1 to 5, g2 represents an integer of 0 to 20, and g3 represents an integer of 0 to 20].

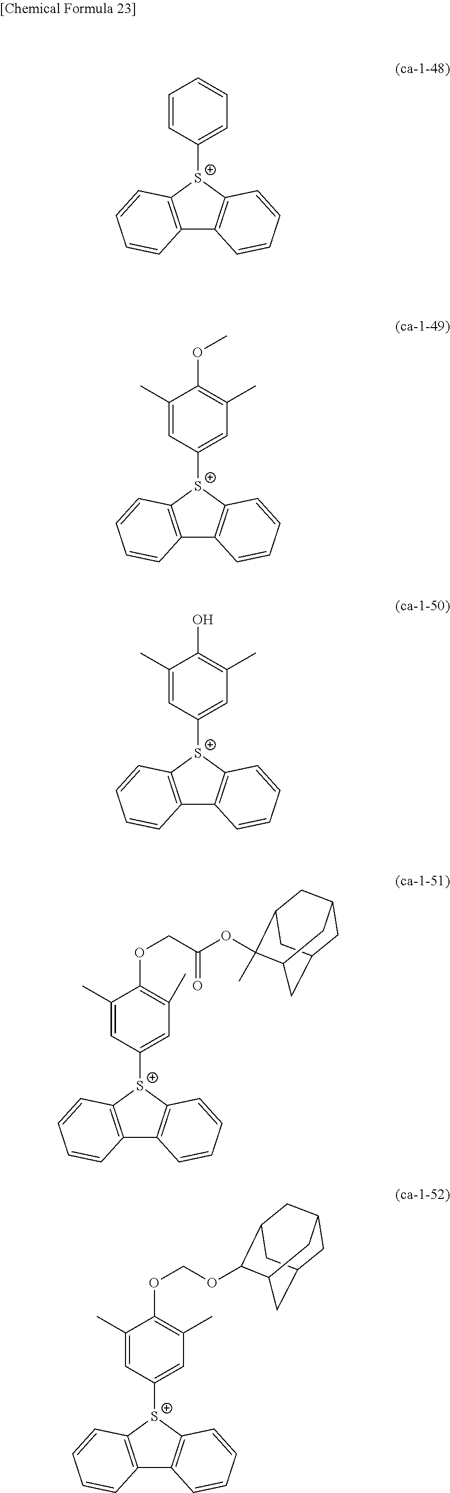 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

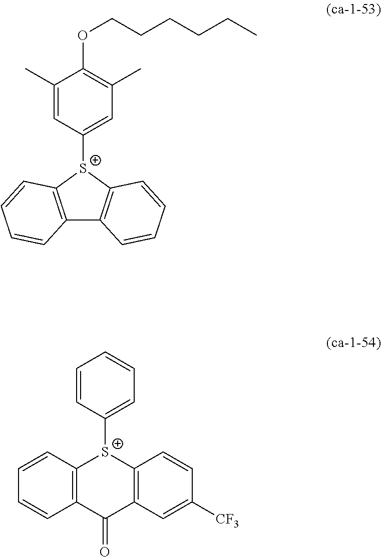 [SMILES: CCCCCCOc1c(C)cc(-[s+]2c3ccccc3c3ccccc32)cc1C ; O=c1c2ccccc2[s+](-c2ccccc2)c2ccc(C(F)(F)F)cc12]

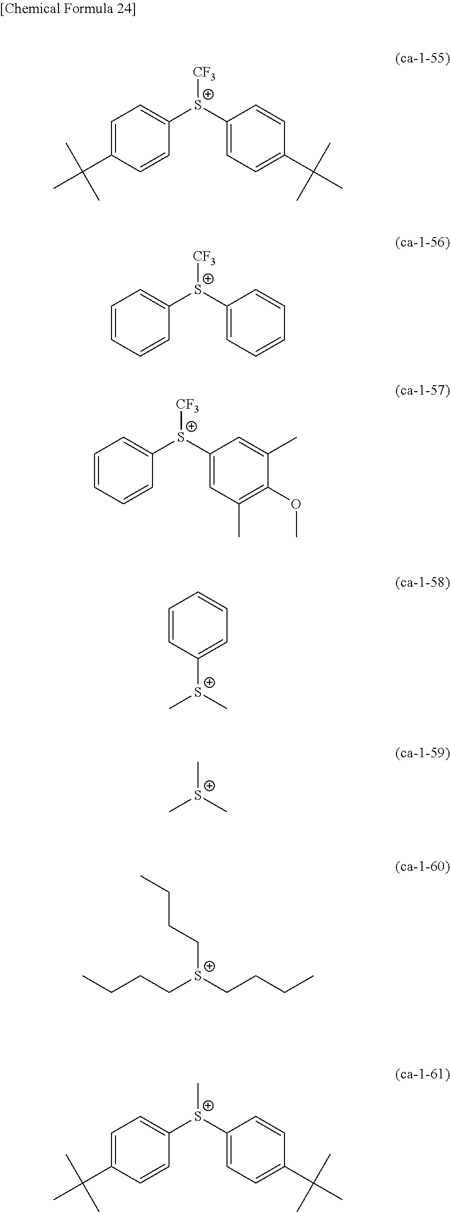

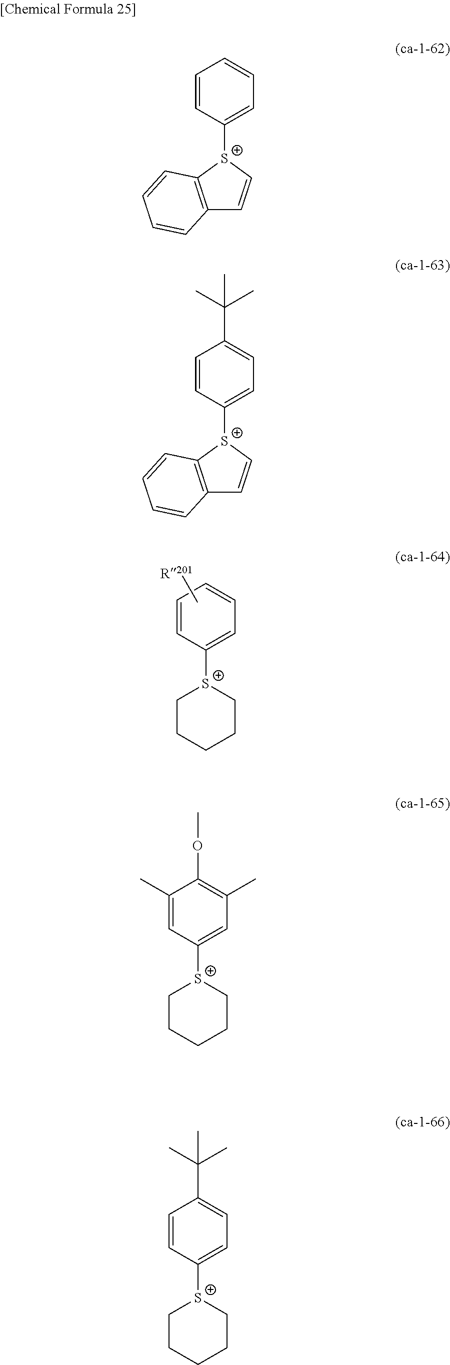

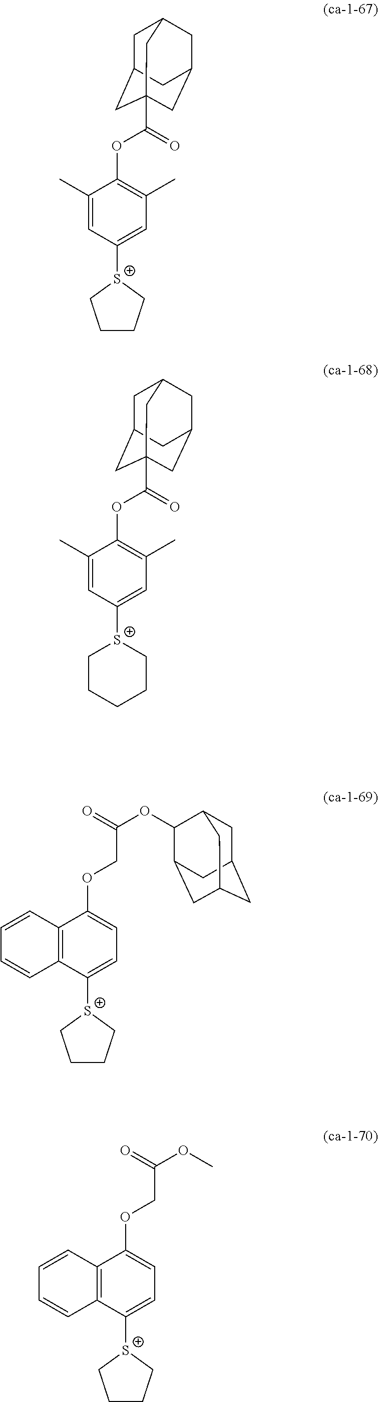 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same group as those for the substituents which may be included in R²⁰¹ to R²⁰⁷ and R²¹⁰ to R²¹²].

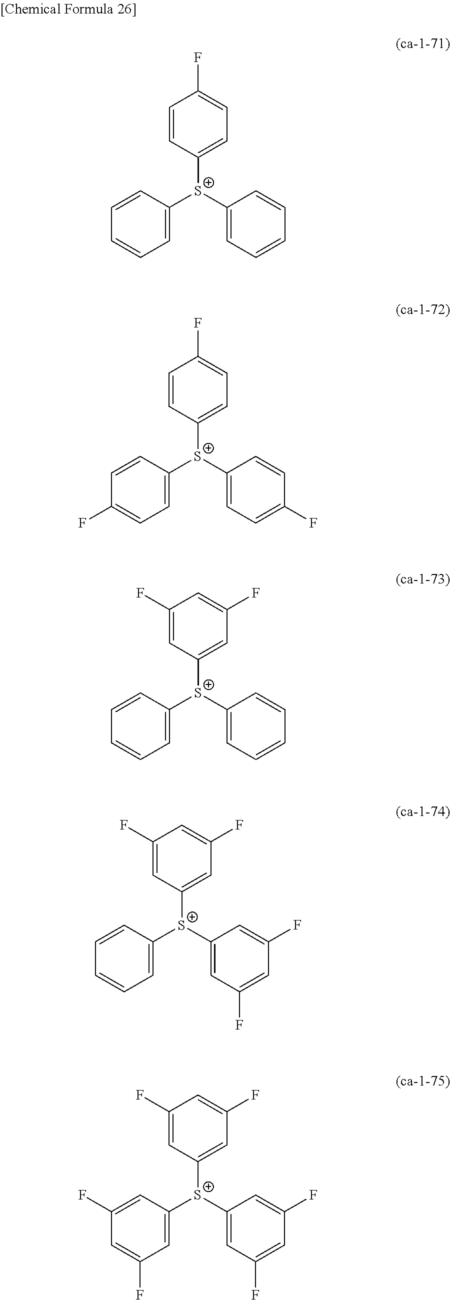 [SMILES: Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1]

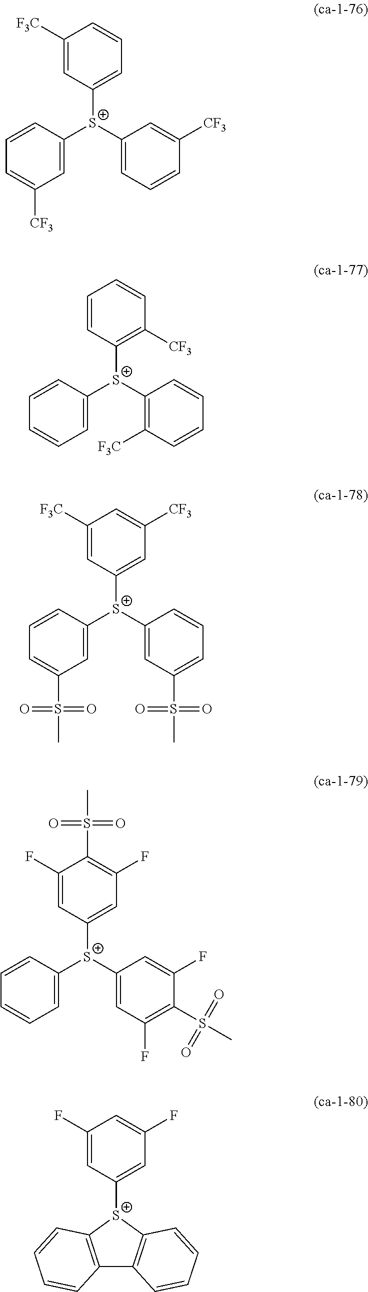 [SMILES: FC(F)(F)c1cccc([S+](c2cccc(C(F)(F)F)c2)c2cccc(C(F)(F)F)c2)c1 ; FC(F)(F)c1ccccc1[S+](c1ccccc1)c1ccccc1C(F)(F)F ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F ; Fc1cc(F)cc(-[s+]2c3ccccc3c3ccccc32)c1]

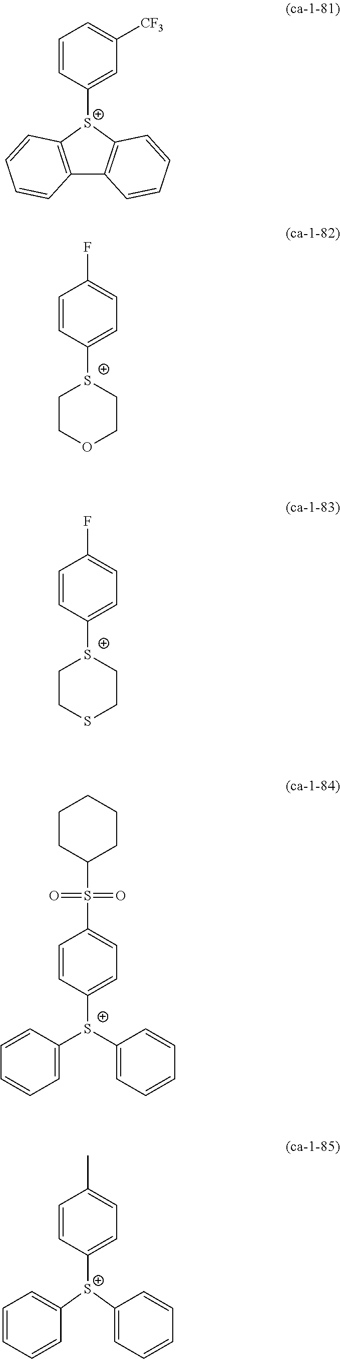 [SMILES: FC(F)(F)c1cccc(-[s+]2c3ccccc3c3ccccc32)c1 ; Fc1ccc([S+]2CCOCC2)cc1 ; Fc1ccc([S+]2CCSCC2)cc1 ; O=S(=O)(c1ccc([S+](c2ccccc2)c2ccccc2)cc1)C1CCCCC1 ; Cc1ccc([S+](c2ccccc2)c2ccccc2)cc1]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

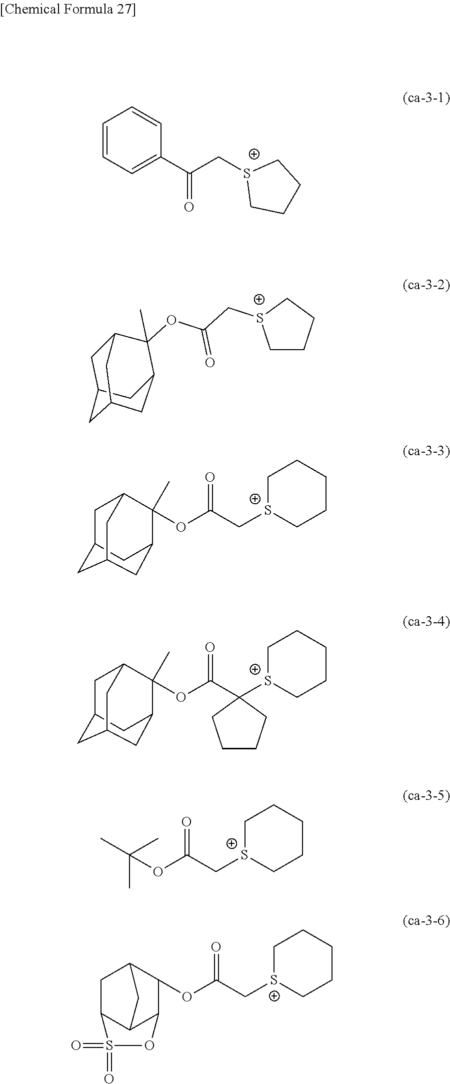 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

As the cation moiety ((M^{m+})_{n0/m}) in Formula (a0-m), a sulfonium cation is preferable, a cation represented by any of Formulae (ca-1) to (ca-3) are more preferable, a cation represented by Formula (ca-1) is still more preferable, and a cation represented by any of Formulae (ca-1-1) to (ca-1-85) is particularly preferable.

In particular, from the viewpoint of increasing the sensitivity, it is preferable that the suitable cation represented by Formula (ca-1) has, as a substituent, an electron-withdrawing group such as a fluorine atom, a fluorinated alkyl group, a sulfonyl group, or an iodine atom, and for example, a cation selected from the group consisting of cations each represented by Chemical Formulae (ca-1-44), (ca-1-71) to (ca-1-84), and (ca-1-85) is s particularly preferable.

Specific examples of the constitutional unit (a0) will be listed below; however, the examples are not limited to these.

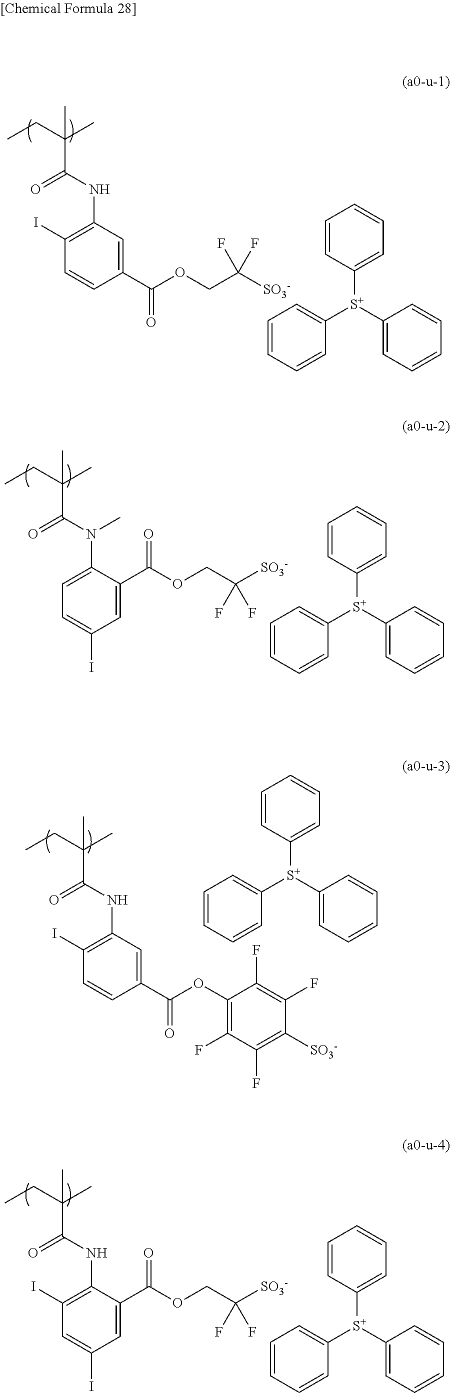

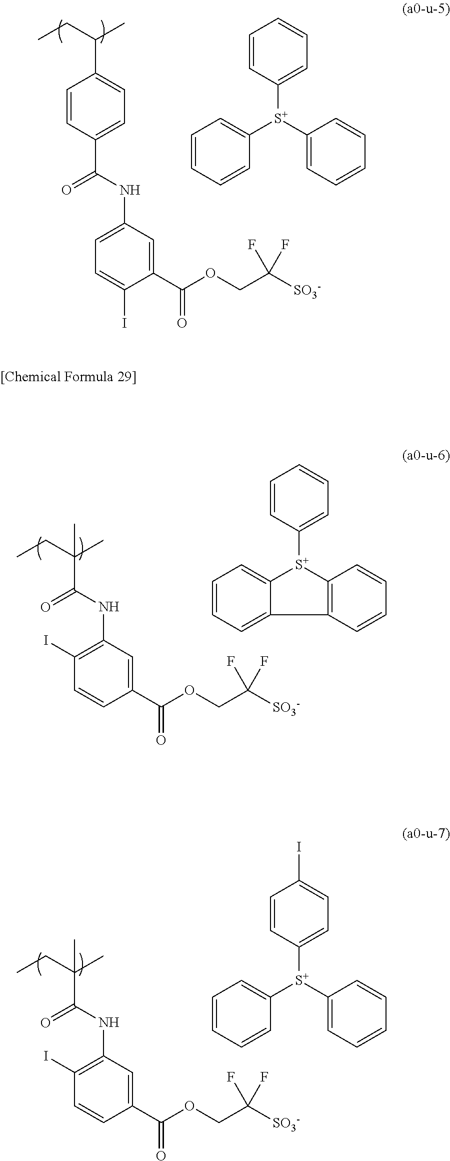

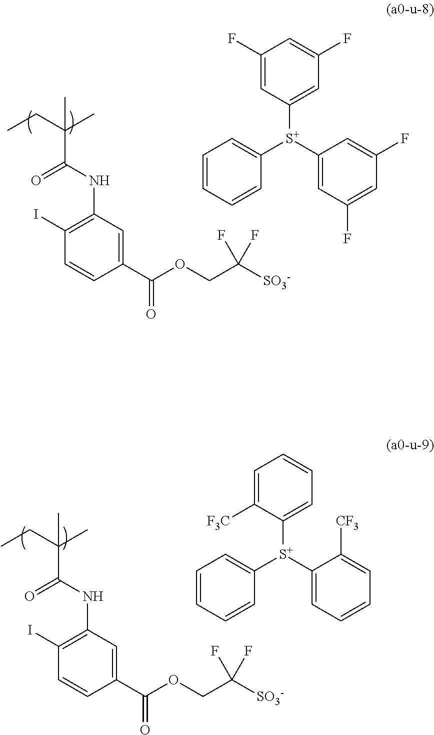

As the constitutional unit (a0), a constitutional unit selected from the group consisting of the constitutional unit represented by General Formula (a0-u1), the constitutional unit represented by General Formula (a0-u2), and the constitutional unit represented by General Formula (a0-u3) is preferable.

Alternatively, from the viewpoint of improving the sensitivity and the lithography characteristics, examples of the preferred constitutional unit (a4) include a constitutional unit in which R^{ar} in General Formula (a0-m) is a benzene ring to which two or more iodine atoms are bonded as substituents.

The constitutional unit (a0) is preferably a constitutional unit represented by any of Formulae (a0-u-1) to (a0-u-9), and from the viewpoint of improving lithography characteristics such as dimensional uniformity, more preferably a constitutional unit represented by any of Formulae (a0-u-4), (a0-u-5), or (a0-u-8).

The constitutional unit (a0) contained in the component (A1) may be one kind or may be two or more kinds.

The content proportion of the constitutional unit a0) in the component (A1) is preferably in a range of 1% to 40% by mole, more preferably in a range of 2% to 30% by mole, still more preferably in a range of 5% to 25% by mole, and particularly preferably in a range of 10% to 20% by mole with respect to the total amount (100% by mole) of all constitutional units constituting the component (A1).

In a case where the proportion of the constitutional unit (a0) is equal to or higher than the lower limit of the above-described preferred range, the lithography characteristics such as sensitivity and dimensional uniformity, and the process margin such as light exposure margin are more easily increased. On the other hand, in a case where the proportion of the constitutional unit. (a0) is equal to or lower than the upper limit of the preferred range described above, balancing with other constitutional units can be easy.

### <<Other Constitutional Units>>

The component (A1) may have other constitutional units as necessary in addition to the constitutional unit (a0) described above.

Examples of the other constitutional units include a constitutional unit (a1) containing an acid decomposable group whose polarity is increased by the action of an acid, a constitutional unit (a10) represented by General Formula (a10-1), a constitutional unit (a5) that generates an acid upon light exposure (here, a constitutional unit corresponding to the constitutional unit (a0) is excluded); a constitutional unit (a6) having acid diffusion controllability; a constitutional unit (a2) containing a lactone-containing cyclic group; and a constitutional unit (a8) derived front a compound represented by General Formula (a8-1).

In regard to constitutional unit (a1):

The constitutional unit (a1) is a constitutional unit that contains an acid decomposable group whose polarity is increased by the action of an acid.

Examples of the acid dissociable group are the same as those which have been suggested as the acid dissociable groups of the base resin for a chemically amplified resist composition.

Specific examples of the suggested acid dissociable group of the base resin for a chemically amplified resist composition include “acetal type acid dissociable group”, “tertiary alkyl ester type acid dissociable group”, “tertiary alkyloxycarbonyl acid dissociable group”, and “secondary alkyloxycarbonyl acid dissociable group” described below.

Acetal type acid dissociable group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

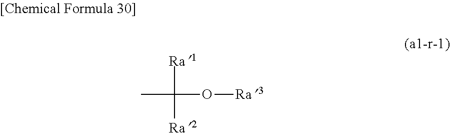

[In the formula, Ra′¹ and Ra′² represent a hydrogen atom or an alkyl group. Ra′³ represents a hydrocarbon group, and Ra′³ may be bonded to any of Ra′¹ and Ra′² to form a ring].

In Formula (a1-r-1), it is preferable that at least one of Ra′¹ and Ra′² represents a hydrogen atom and more preferable that both Ra′¹ and Ra′² represent a hydrogen atom.

In a case where Ra′¹ or Ra′² represents an alkyl group, examples of the alkyl group include the same alkyl groups described as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene), and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—, OH, —R^{P2}—CN, and —R^{P2}—COOH (hereinafter, these substituents will also be collectively referred to as “Ra′⁵”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02.6] decanyl group, a tricyclo[3.3.1.13,7] decanyl group, a tetracyclo[6.2.1.13.6.02.7] dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

Tertiary alkyl ester type acid dissociable group:

Examples of the acid dissociable group that protects a carboxy group among the polar groups include an acid dissociable group represented by General Formula (a1-r-2).

Among examples of the acid dissociable group represented by Formula (a1-r-2), hereinafter, a group formed of an alkyl group will also be referred to as “tertiary alkyl ester type acid dissociable group” for convenience.

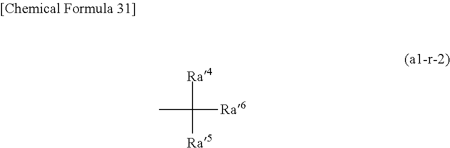

[In the formula, Ra′⁴ to Ra′⁶ beach represent a hydrocarbon group, and Ra′⁵ and Ra′⁶ may be bonded to each other to form a ring].

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to each other, suitable examples thereof include a group represented by General Formula (a1-r2-4).

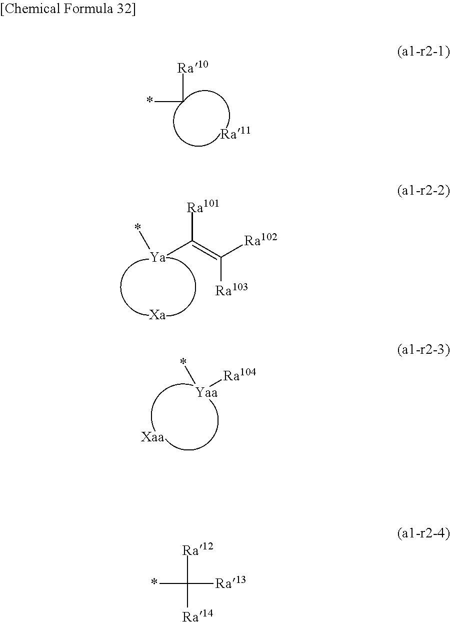

[In Formula (a1-r2-1). Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2). Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms. Some or all hydrogen atoms contained in the chain-like saturated hydrocarbon group may be substituted. Ra′¹⁴ represents a hydrocarbon group which may have a substituent. * represents a bonding site (the same applies hereinafter)].

In Formula (a1-r2-1), Ra′¹⁹ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 cardon atoms, and particularly preferably 1 to 5 carbon atoms.

Examples of the branched alkyl group as Ra′¹⁰ include the same groups as those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O), —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02.6] decanyl group, a tricyclo[3.3.1.13,7] decanyl group, a tetracyclo[6.2.1.13,6.02.7]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same groups as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methyleyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1, is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms. Examples of the chain-like monovalent saturated hydrocarbon group having 1 to 10 carbon atoms as Ra′¹² and Ra′¹³ include the same one as the chain-like monovalent saturated hydrocarbon group having 1 to 10 carbon atoms as Ra¹⁰¹ to Ra¹⁰³ as described above. Some or all hydrogen atoms contained in the chain-like saturated hydrocarbon group may be substituted.

Ra′¹³ and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent include the same groups as those for Ra^{x5} described above.

In Formula (a1-r24), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atom, have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same group, as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be any of the 1-position or the 2-position of the naphthyl group.

In a case where Ra′¹⁴ in Formula (a1-r2-4) represents an anthryl group, the position bonded to the tertiary carbon atom in Formula (a1-r2-4) may be any of the 1-position, the 2-position, or the 9-position of the anthryl group.

Specific examples of the group represented by Formula (a1-r2-1) are shown below.

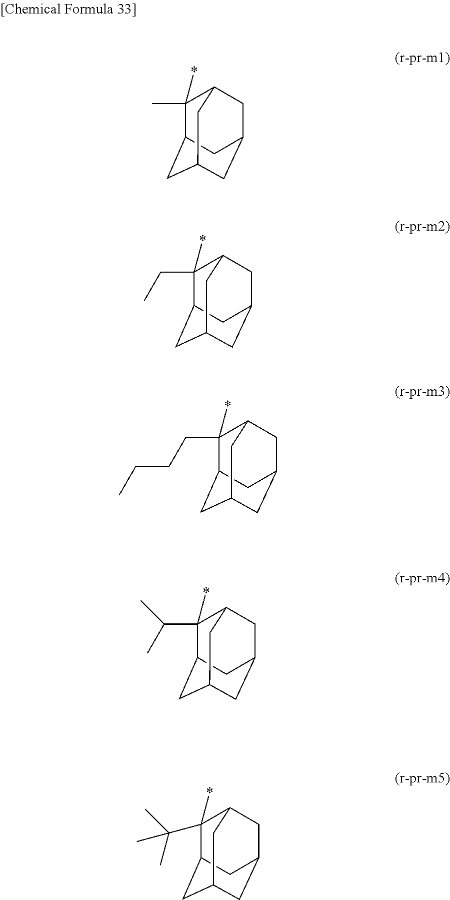 [SMILES: *C1(C)C2CC3CC(C2)CC1C3 ; *C1(CC)C2CC3CC(C2)CC1C3 ; *C1(CCCC)C2CC3CC(C2)CC1C3 ; *C1(C(C)C)C2CC3CC(C2)CC1C3 ; *C1(C(C)(C)C)C2CC3CC(C2)CC1C3]

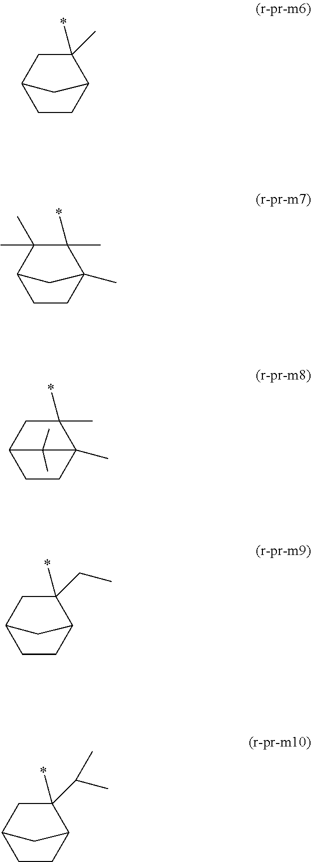

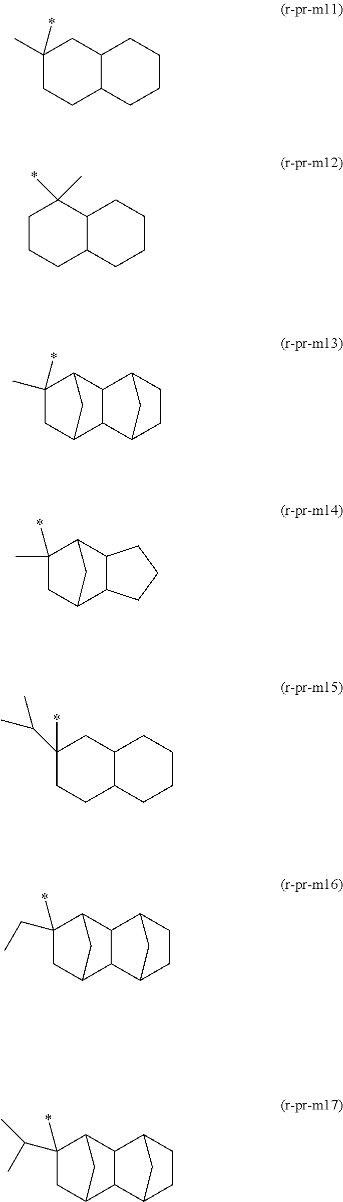

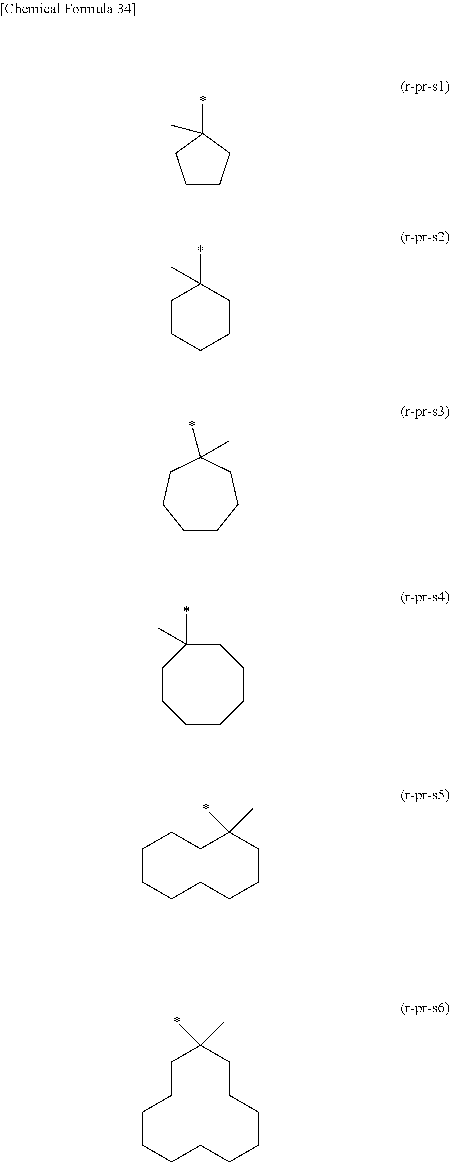

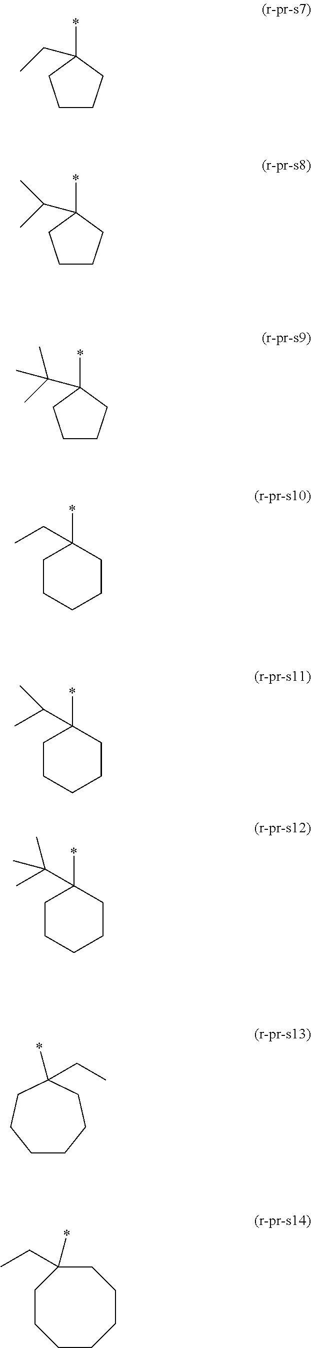

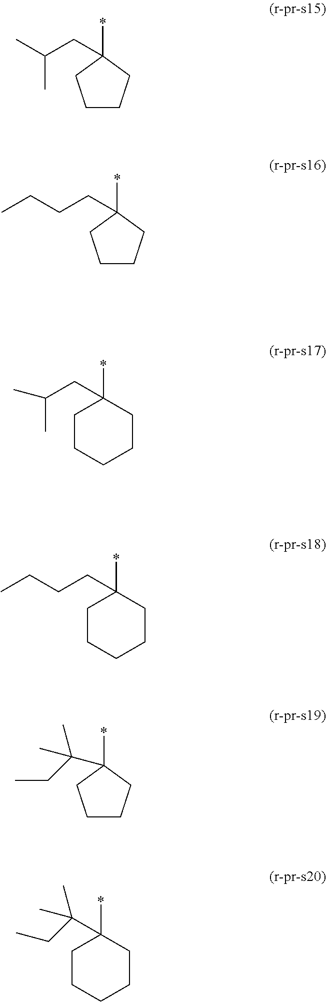

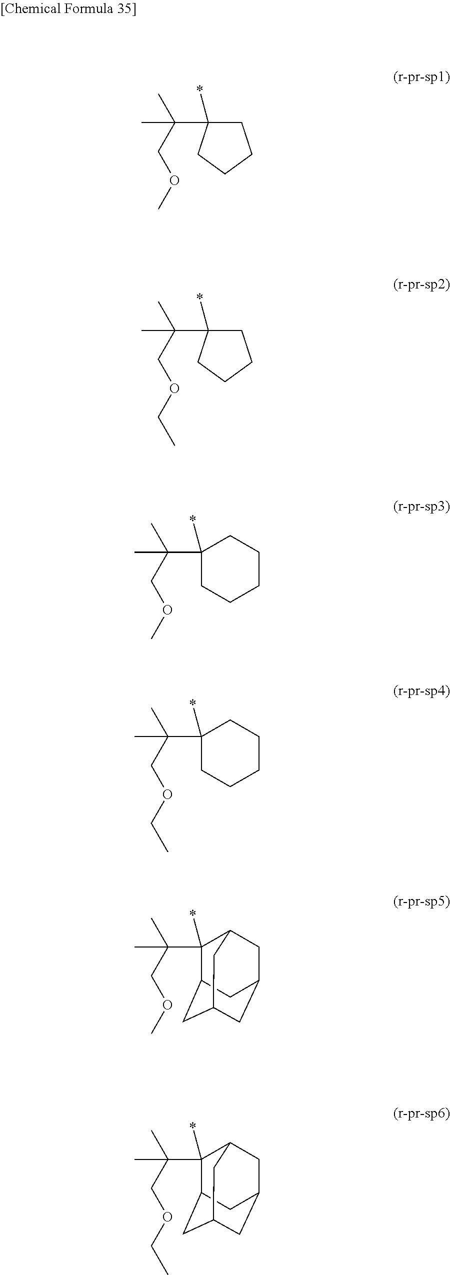 [SMILES: *C1(C(C)(C)COC)CCCC1 ; *C1(C(C)(C)COCC)CCCC1 ; *C1(C(C)(C)COC)CCCCC1 ; *C1(C(C)(C)COCC)CCCCC1 ; *C1(C(C)(C)COC)C2CC3CC(C2)CC1C3 ; *C1(C(C)(C)COCC)C2CC3CC(C2)CC1C3]

Specific examples of the group represented by Formula (a1-r2-2) are shown below.

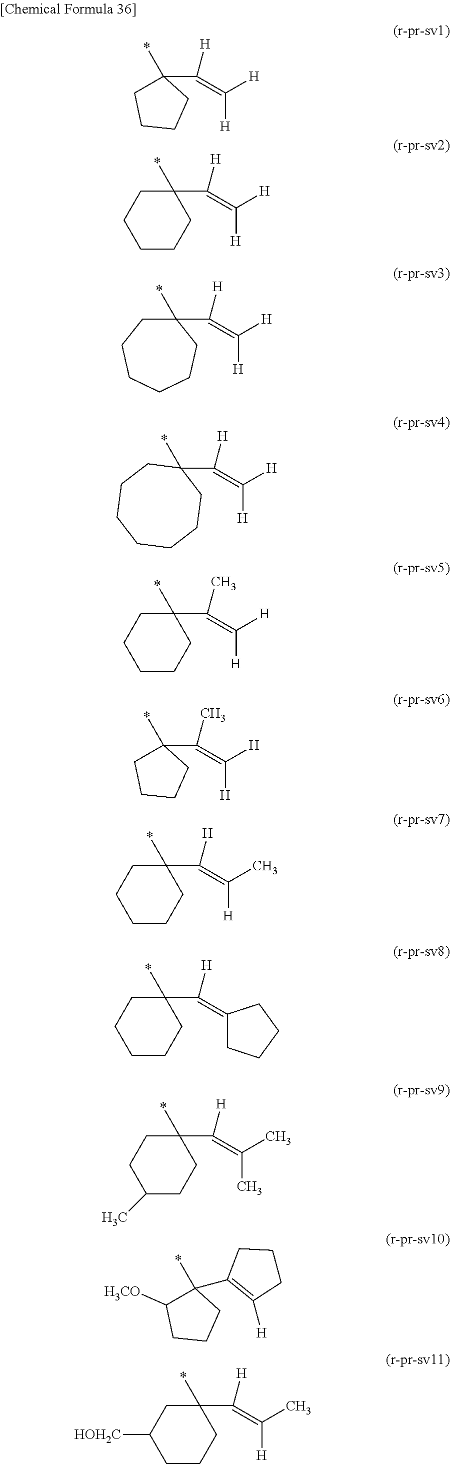

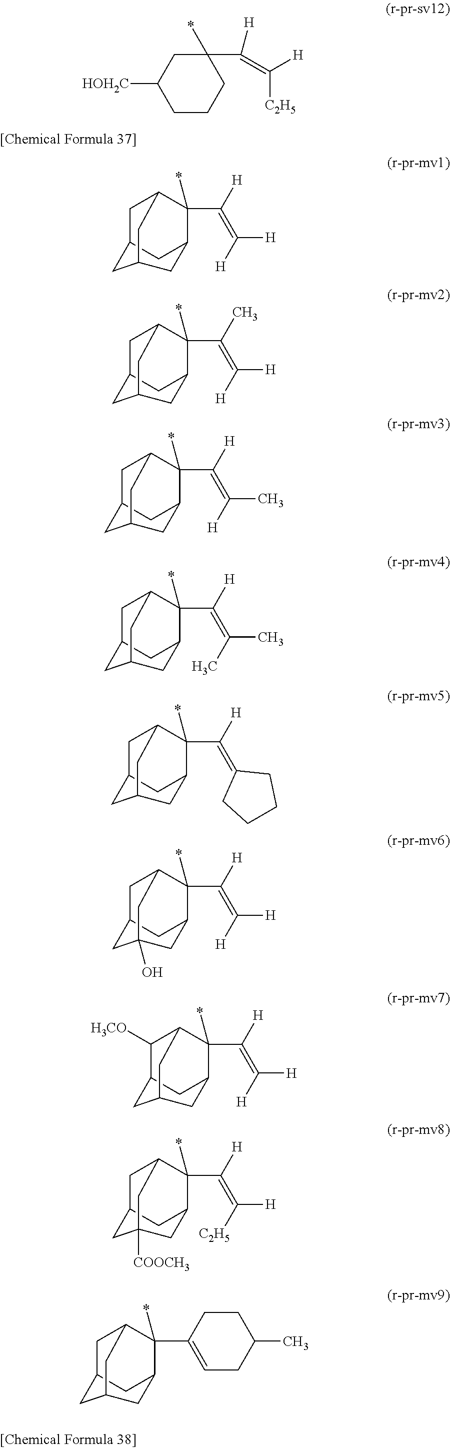

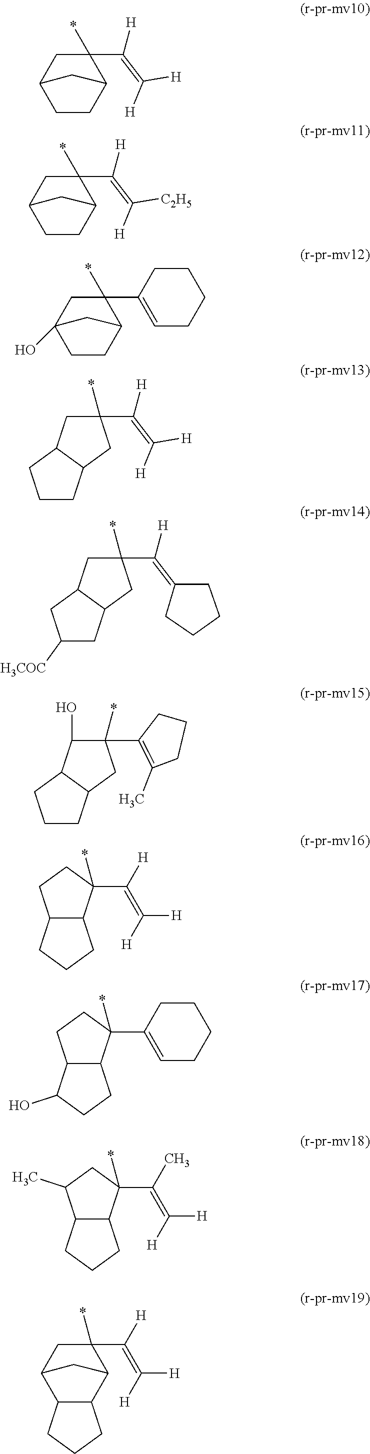

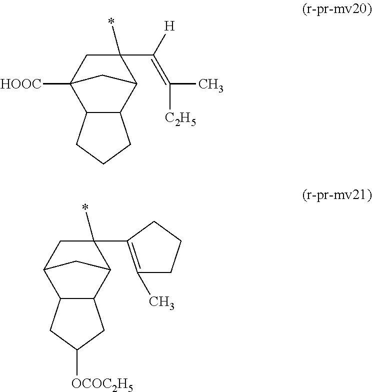

Specific examples of the group represented by Formula (a1-r2-3) are shown below.

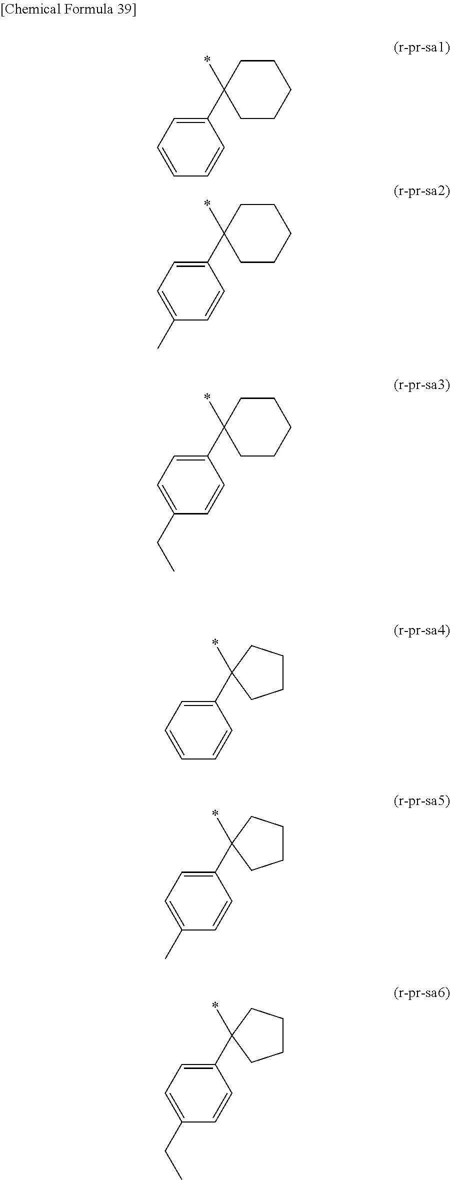

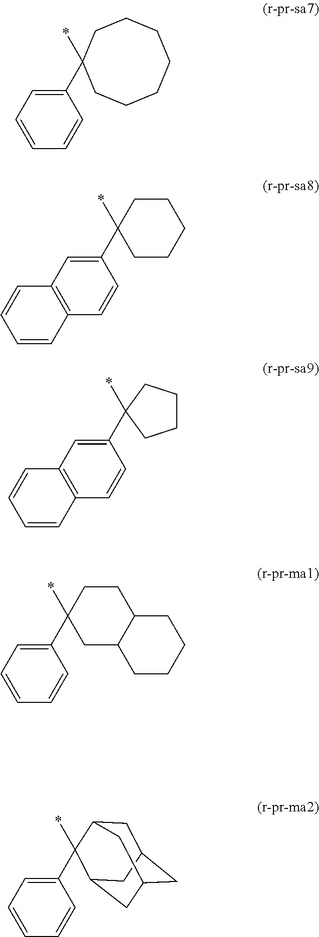 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

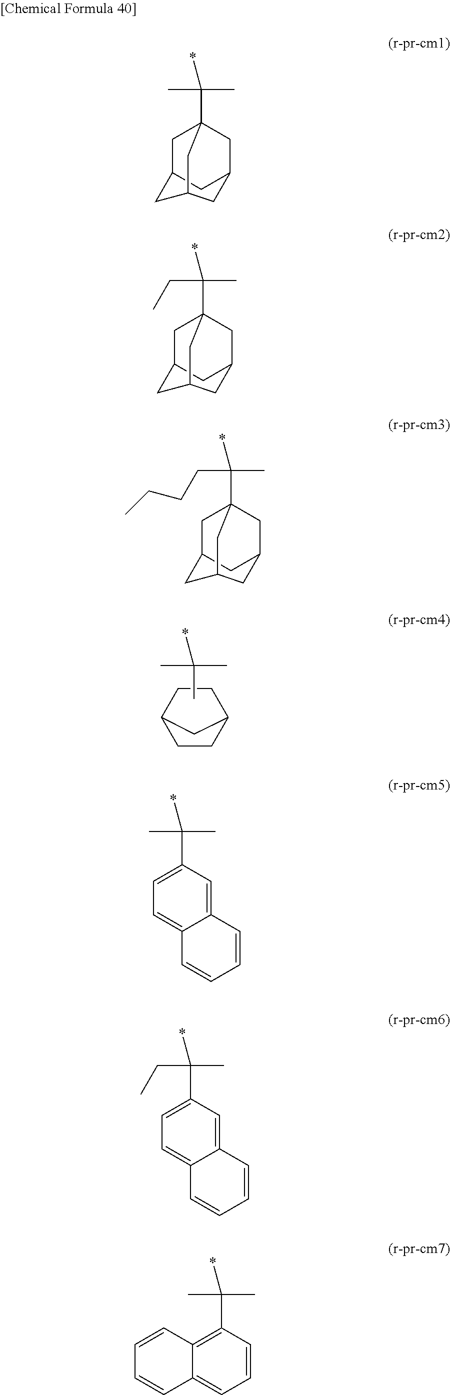

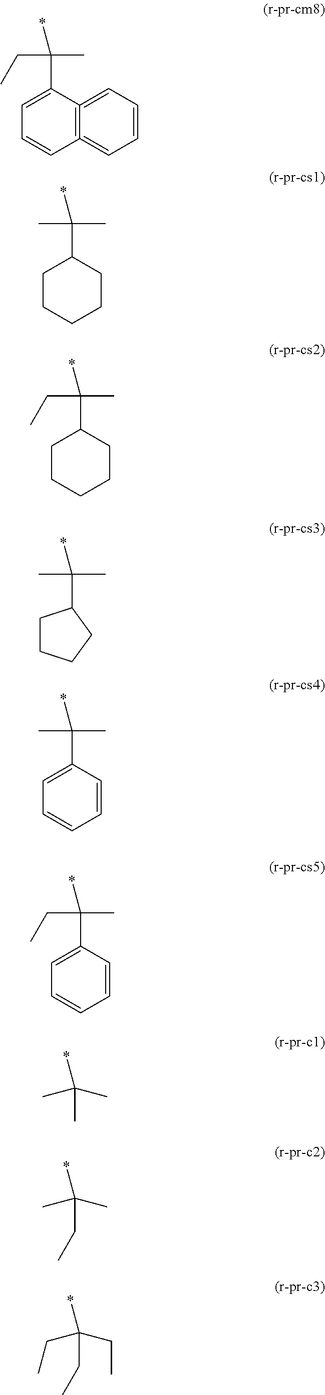

Tertiary alkyloxycarbonyl acid dissociable group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

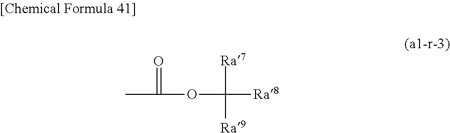

[In the formula, Ra′⁷ to Ra′⁹ each represent an alkyl group].

In Formula (a1-r-3). Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Secondary alkyl ester type acid dissociable group:

Examples of the acid dissociable group that protects a carboxy group among the polar groups include an acid dissociable group represented by General Formula (a1-r-4).

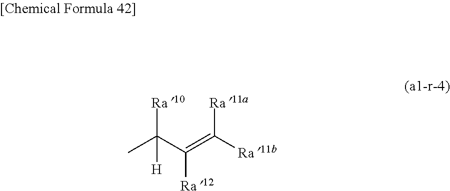

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may e bonded to each other to form a ring. Ra′^{11a} or Ra′^{11b} and Ra′¹² may be bonded to each other to form a ring].

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra′⁵ described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the same rings as those formed by Ra′¹⁰ and Ra′^{11a} or Ra′^{11b} being bonded to each other.

Specific examples of the group represented by Formula (a1-r-4) are shown below.

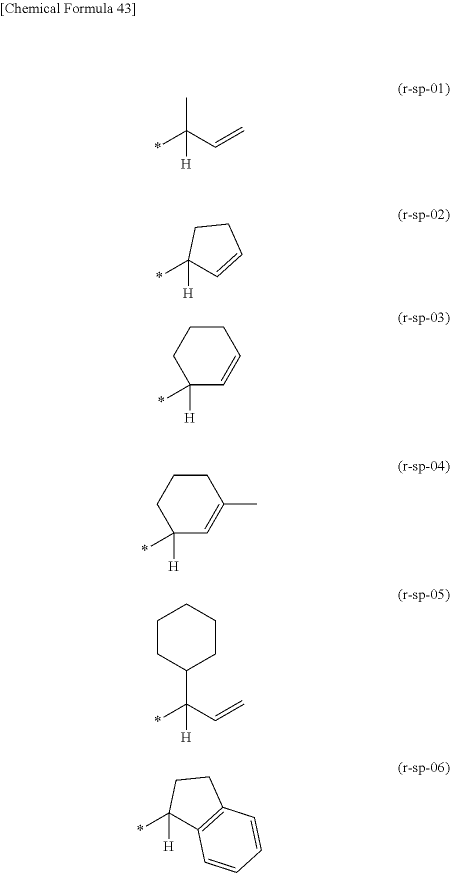

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Anong the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include a constitutional unit represented by General Formula (a1-1), (a1-2), or (a1-3).

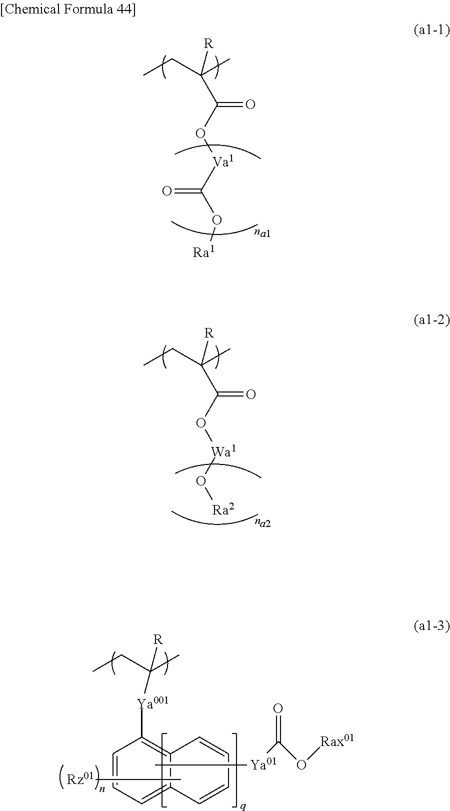

[In the formula, R represents a hydrogen atom, an alkyl group having 1 to 5 carbon atoms, or a halogenated alkyl group having 1 to 5 carbon atoms. Va¹ represents a divalent hydrocarbon group which may contain an ether bond, nₐ₁ represents an integer of 0 to 2. Ra¹ represents an acid dissociable group represented by General Formula (a1-r-1), (a1-r-2), or (a1-r-4). Wa¹ represents an (nₐ₂+1)-valent hydrocarbon group, nₐ₂ represents an integer of 1 to 3. Ra² represents an acid dissociable group represented by General Formula (a1-r-1) or (a1-r3). Ya⁰⁰¹ represents a single bond or a divalent linking group. Ya⁰¹ represents a single bond or a divalent linking group. Rax⁰¹ represents an acid dissociable group represented by General Formula (a1-r-1), (a1-r-2), or (a1-r-4). Rz⁰¹ represents an alkyl group, a halogen atom, a halogenated alkyl group, a hydroxy group, or an alkoxy group, q represents an integer of 0 to 3, n represents an integer of 0 or greater. Here, n≤q×2+4 is satisfied].

In Formulae (a1-1) to (a1-3), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂, —C(CH₃)(CH₂CH₃)—, —C(CH₃)(CH₂CH₂CH₃)—, and —C(CH₂CH₃)₂; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group), and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents preferably an acid dissociable group represented by General Formula (a1-r-2) or (a1-r-4) and, among these, more preferably a group represented by General Formula (a1-r2-1) or an acid dissociable group represented by General Formula (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), it is preferable that Ra² represents an acid dissociable group represented by General Formula (a1-r-1).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰⁰¹ represents an ester bond [—C(═O)—O— or —O—C(═O)—], an ether bond (—O—), a linear or branched alkylene group, an aromatic hydrocarbon group, a combination thereof, or a single bond. The alkylene group has preferably 1 to 10 carbon atoms, more preferably 1 to 6 carbon atoms, still more preferably 1 to 4 carbon atoms, and particularly preferably 1 to 3 carbon atoms.

Among these, Ya⁰⁰¹ represents more preferably a combination of an ester bond [—C(═O)—O— or —O—C(═O)—] and a linear alkylene group, or a single bond and still more preferably a single bond.

In Formula (a1-3), the divalent linking group as Ya⁰¹ is not particularly limited, and suitable examples thereof include a divalent hydrocarbon group which may have a substituent and a divalent linking group having a heteroatom.

Among these, it is preferable that Ya⁰¹ represents an ester bond [—C(═O)—O— or —O—C(═O)—], an ether bond (—O—) a linear or branched alkylene group, an aromatic hydrocarbon group, a combination thereof, or a single bond. Among these, Ya⁰¹ is more preferably a combination of an ester bond [—C(═O)—O— or —O—C(═O))—] and a linear alkylene group, or a single bond, and still more preferably a single bond.

In Formula (a1-3), Rax⁰¹ represents preferably an acid dissociable group represented by General Formula (a1-r-2) or (a1-r-4) and, among these, more preferably an acid dissociable group represented by General Formula (a1-r-2) and still more preferably a group represented by General Formula (a1-r2-1).

In Formula (a1-3), the alkyl group, the halogenated alkyl group, and the alkoxy group as Rz⁰¹ have preferably 1 to 10 carbon atoms, more preferably 1 to 5 carbon atoms, still more preferably 1 to 3 carbon atoms, and particularly preferably 1 or 2 carbon atoms. The alkyl group, the halogenated alkyl group, and the alkoxy group may be linear or branched.

As the halogen atom as Rz⁰¹, an iodine atom or a bromine atom is preferable. As the halogen atom of the halogenated alkyl group represented by Rz⁰¹, a fluorine atom, an iodine atom, or a bromine atom is preferable, and a fluorine atom is more preferable.

Rz⁰¹ represents preferably an alkoxy group or a hydroxy group and more preferably a hydroxy group.

In Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In Formula (a1-3), n represents an integer of 0 or greater, preferably an integer of 0 to 5, more preferably an integer of 0 to 3, and still more preferably 1 or 2. In a case where n represents an integer of 2 or greater, two or more Rz⁰¹'s may be the same as or different from each other.

In Formula (a1-3), n≤q×2+4 is satisfied. For example, in a case where q represents 1 and a naphthalene structure is formed, all six hydrogen atoms of the naphthalene may be substituted with Rz⁰¹'s. In addition, in the naphthalene, the substitution positions of Ya⁰⁰¹, a -Ya⁰¹-C(═O)—O—Ra⁰¹ group, and Rz⁰¹ are not particularly limited.

Specific examples of the constitutional unit (a1) are shown below.

In the formulae shown below, R^{α} represents a hydrogen atom, a methyl group, or a trifluoromethyl group. Rz's each independently represent a hydrogen atom, an alkyl group, a halogen atom, a halogenated alkyl group, a hydroxy group, or an alkoxy group.

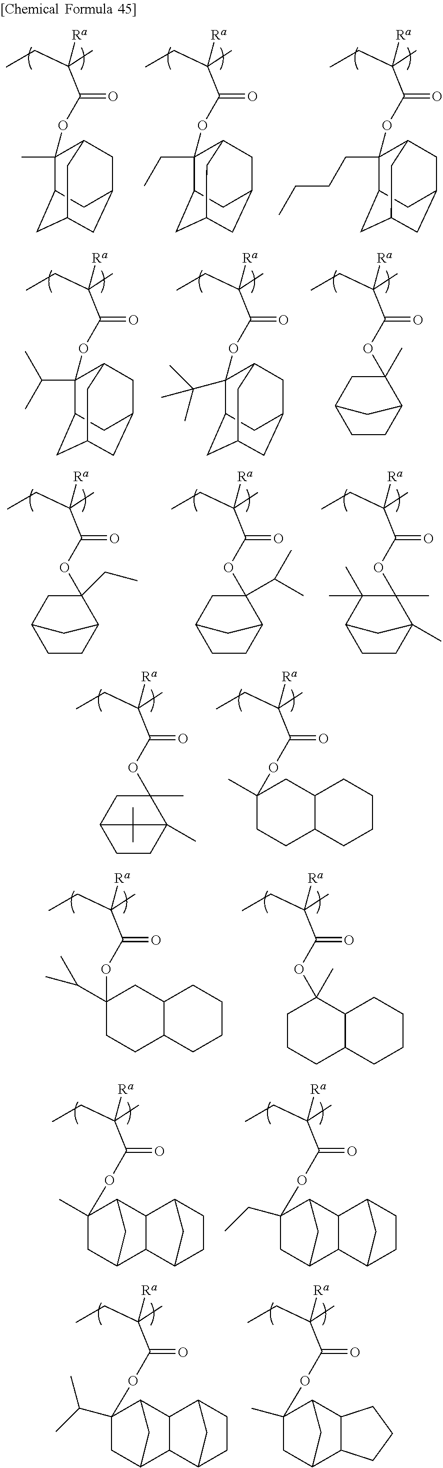

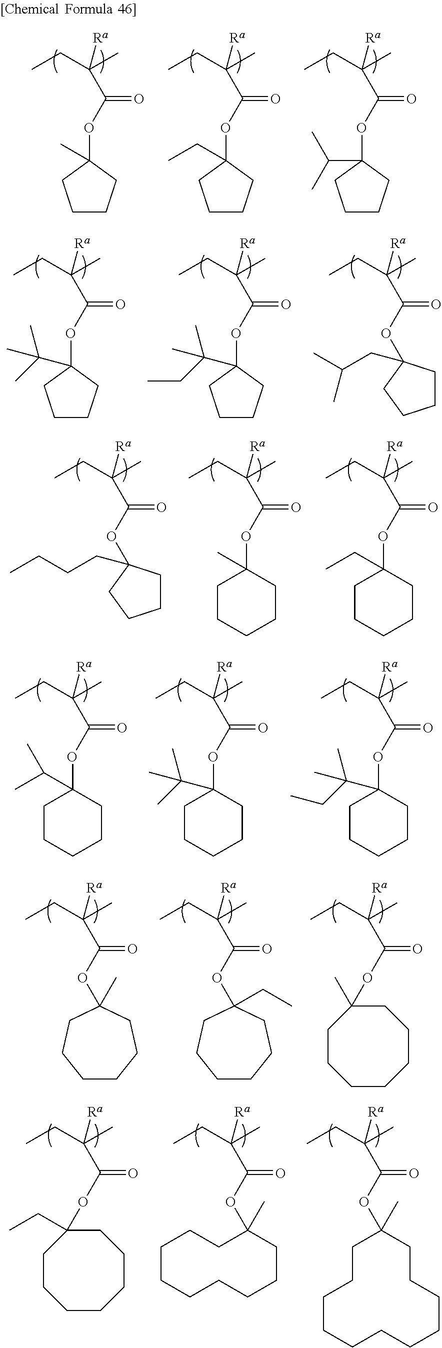

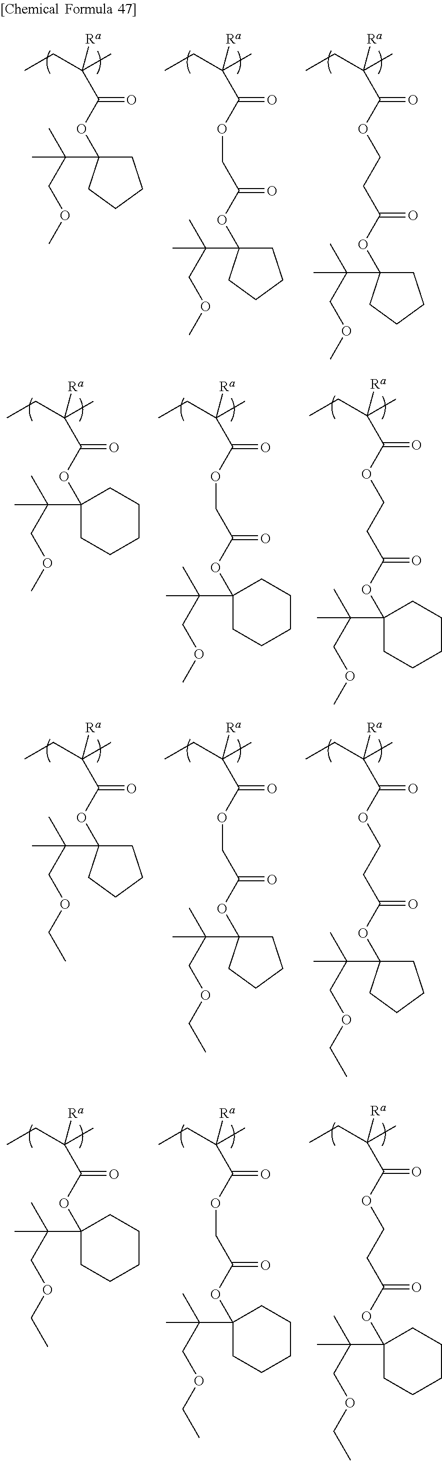

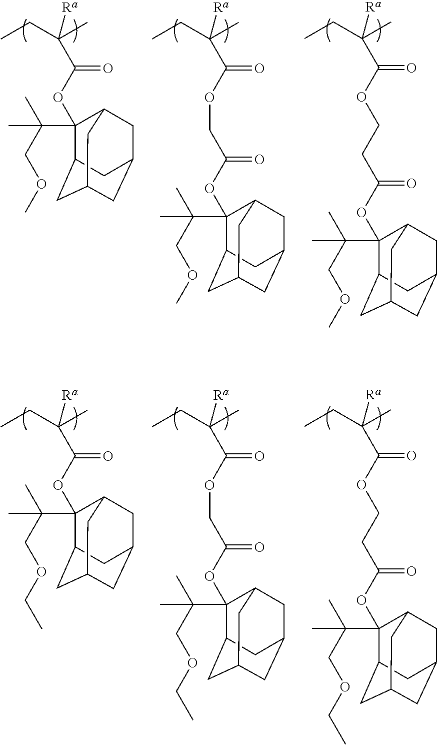

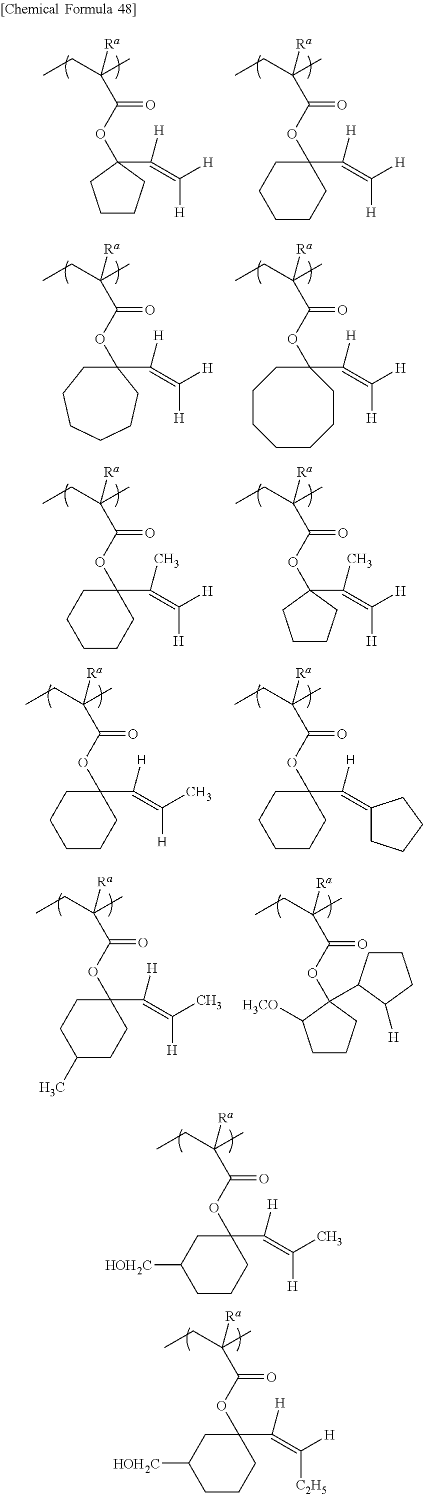

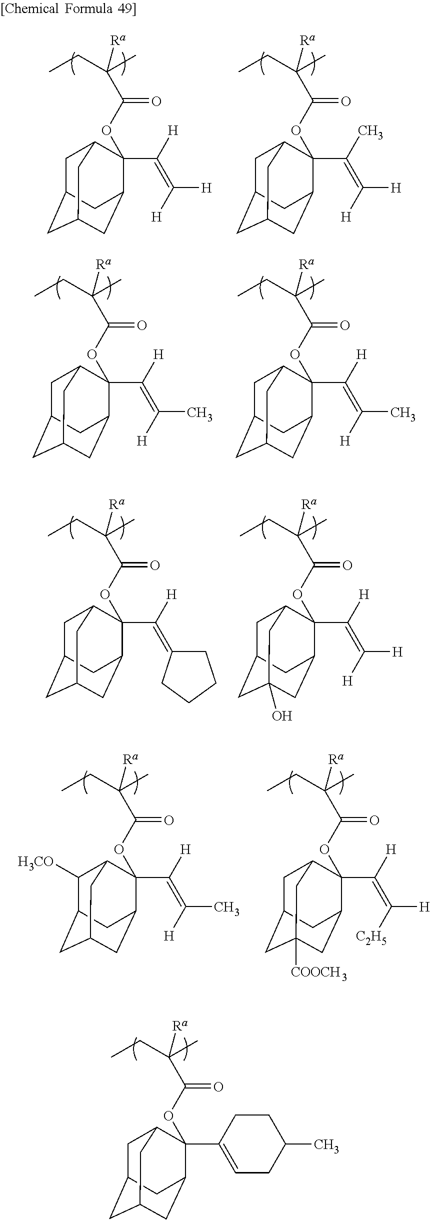

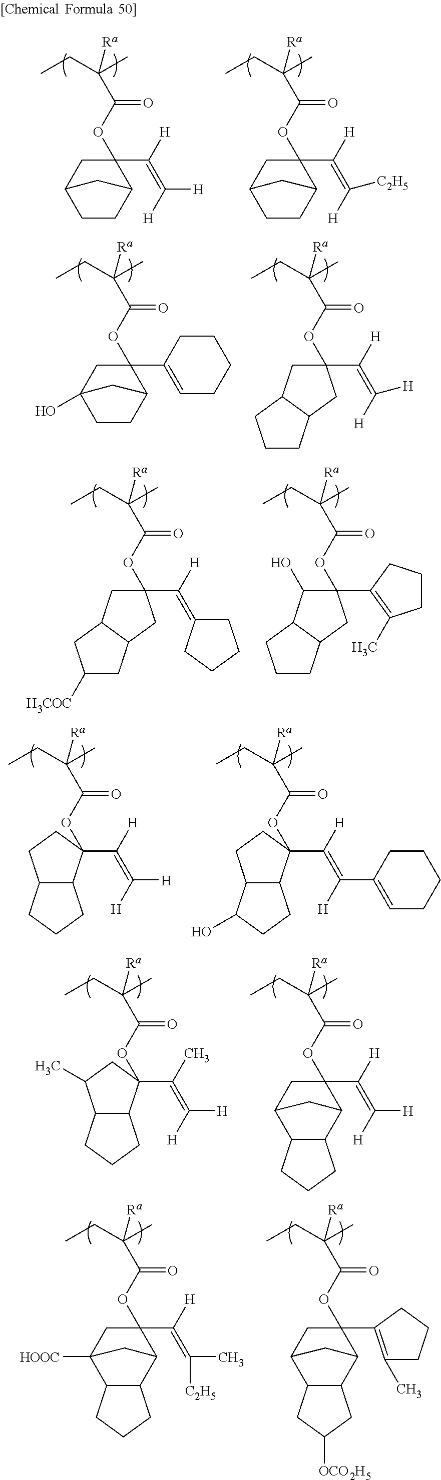

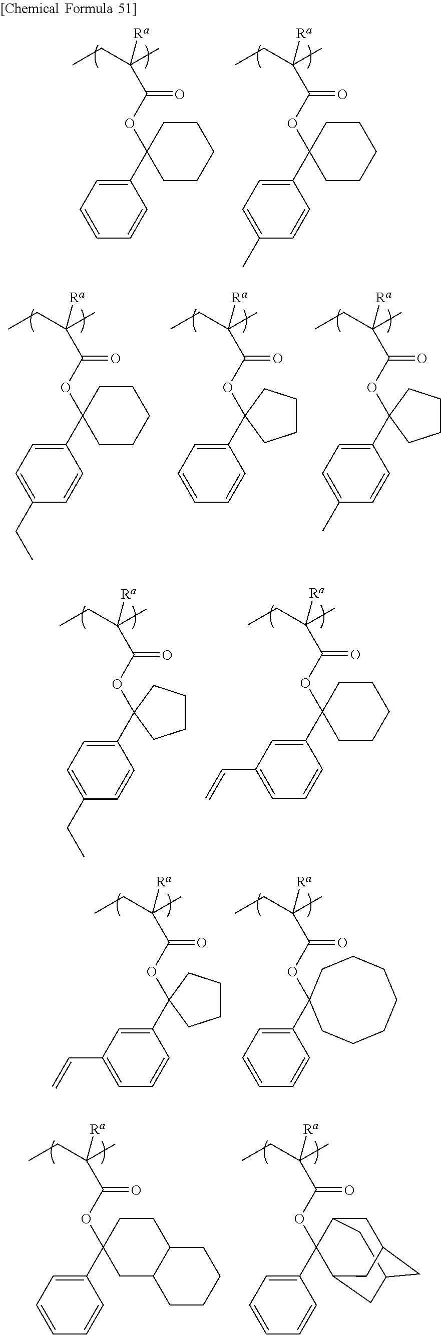

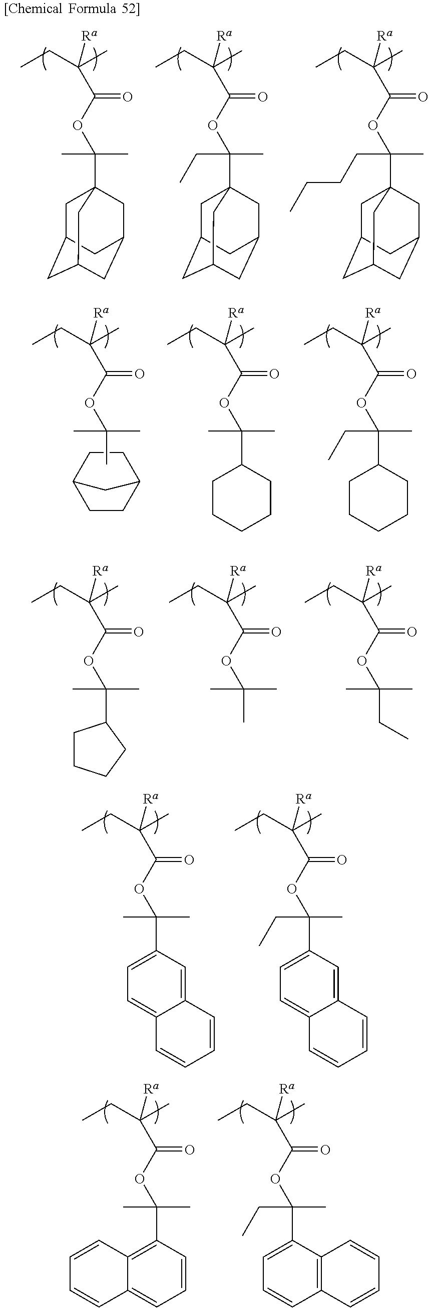

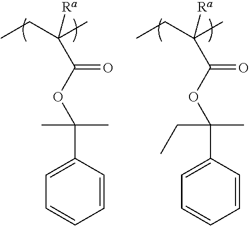

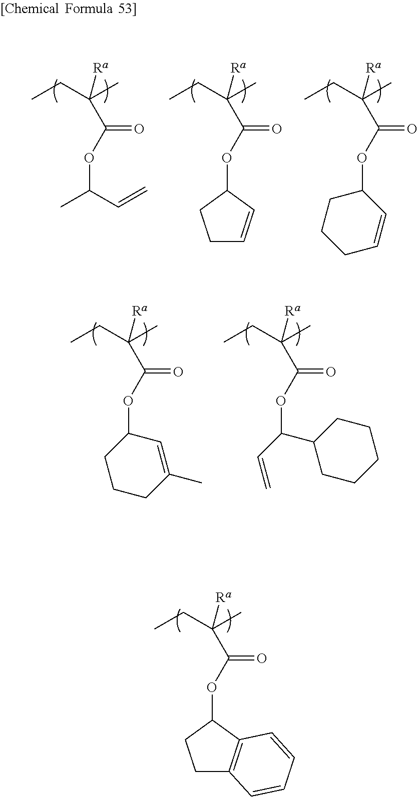

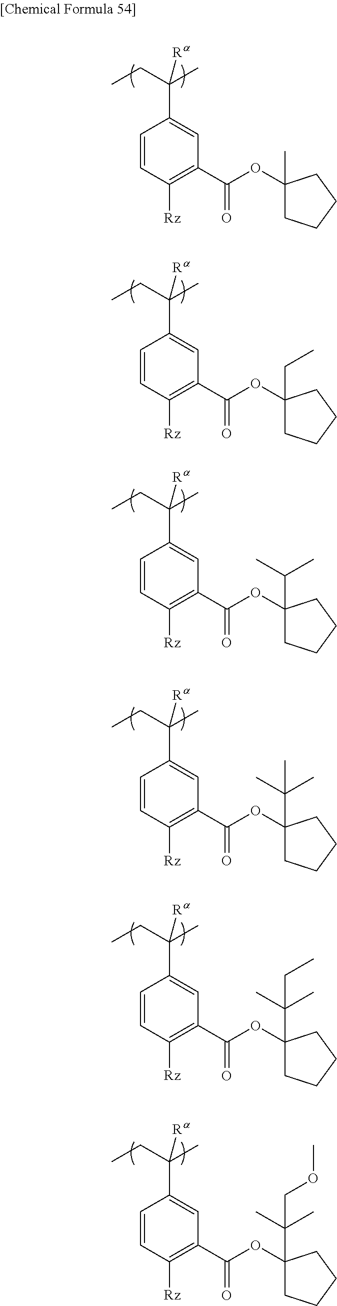

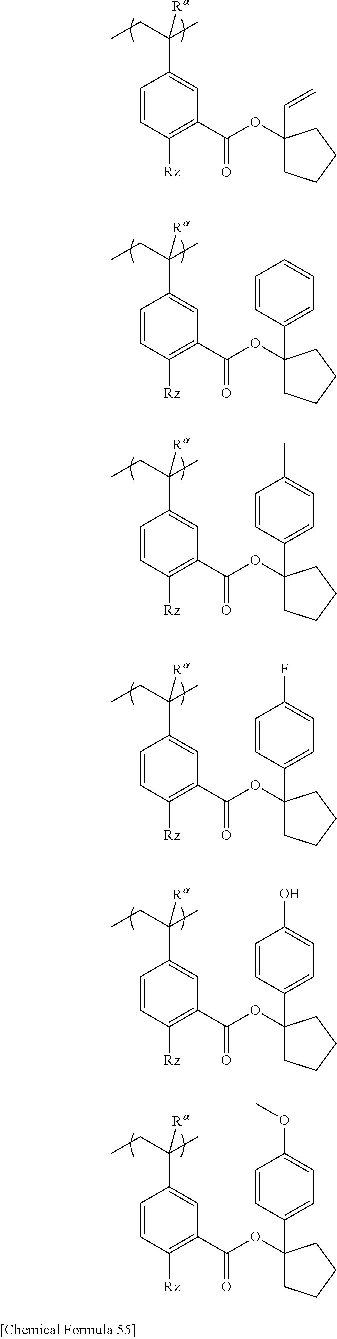

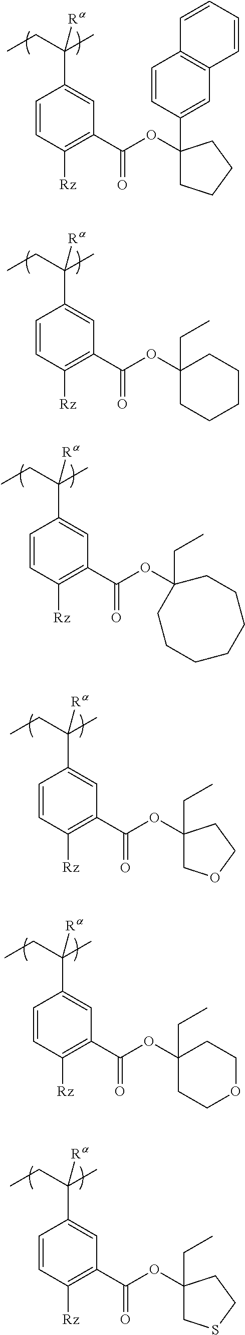

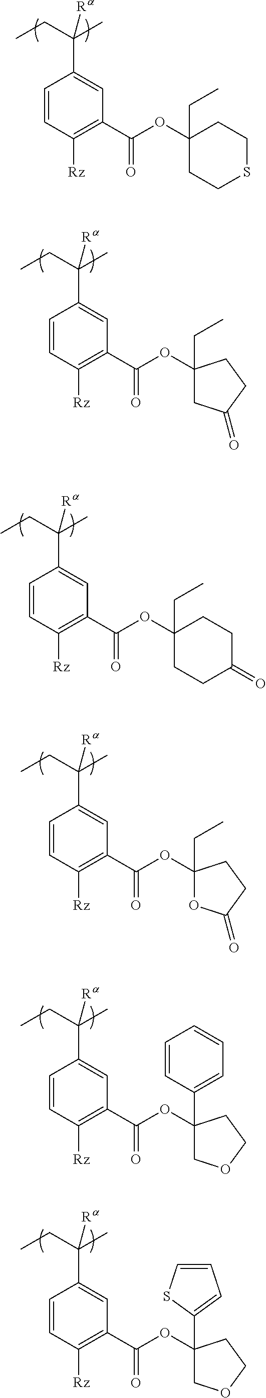

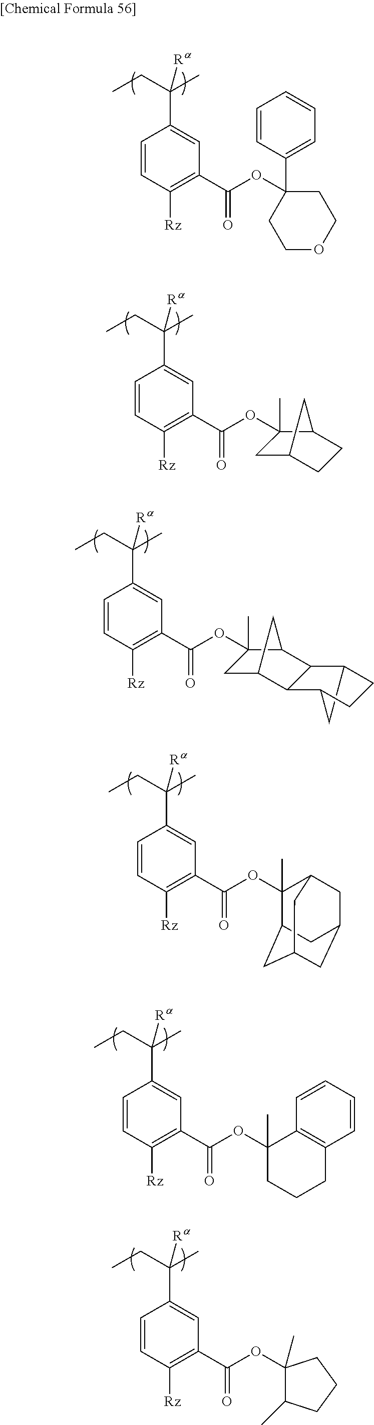

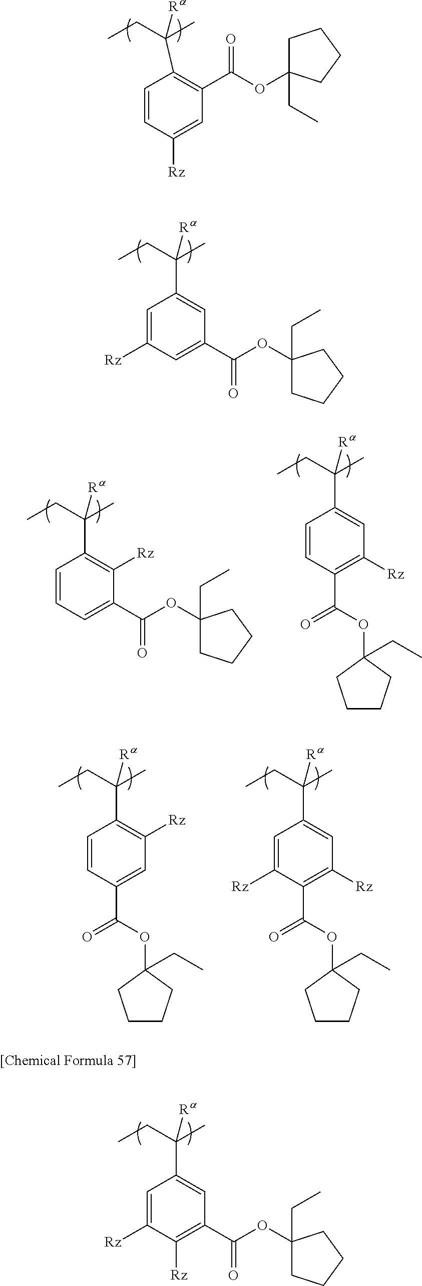

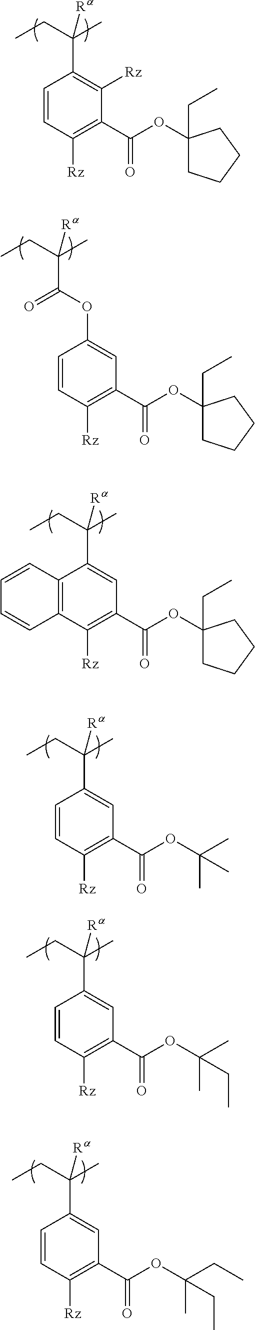

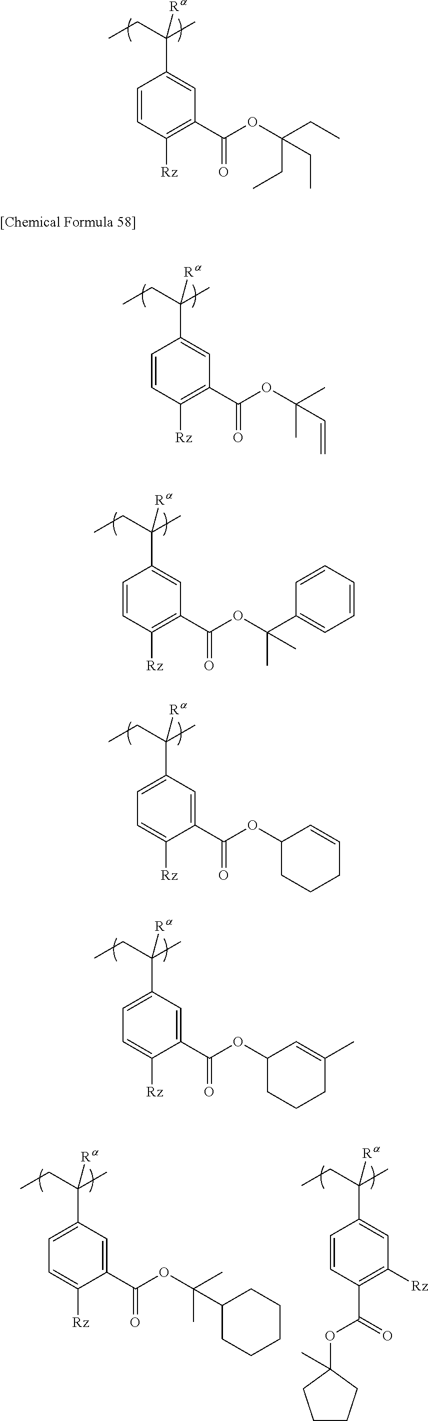

The constitutional unit (a1) included in the component (A1) may be used alone or two or more kinds thereof.

From the viewpoint of further easily enhancing the lithography characteristics (the sensitivity, the shape, and the like) using electron beams or EUV, a constitutional unit represented by Formula (a1-1) or a constitutional unit represented by Formula (a1-3) is more preferable as the constitutional unit (a1).

Among these, from the viewpoint of suitably increasing the reactivity for EB or EUV, the acid dissociable groups (Ra¹, Rax⁰¹) are each preferably an acid dissociable group represented by General Formula (a1-r2-1), (a1-r2-3), (a1-r2-4), or (a1-r-4) and, among these, particularly preferably a cyclic group.

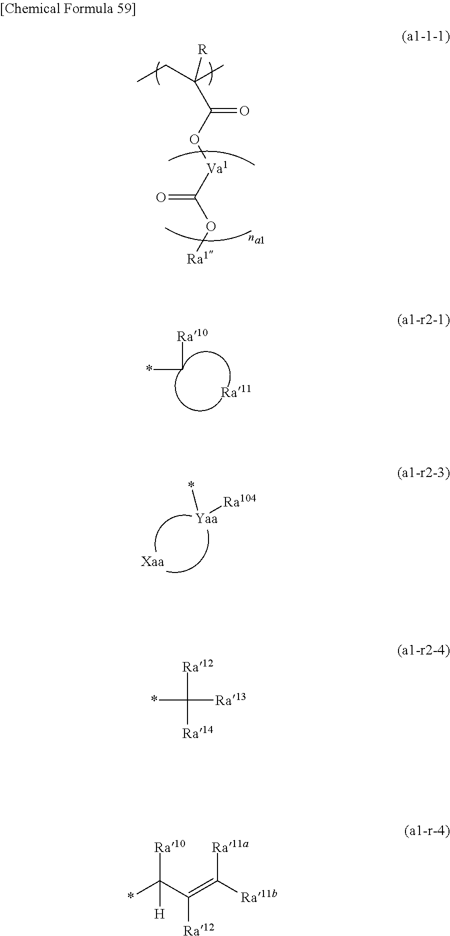

[In the formulae, Ra¹″ represents an acid dissociable group represented by General Formula (a1-r2-1), (a1-r2-3), (a1-r2-4), or (a1-r-4). * represents a bonding site].

In Formula (a1-1-1), R, Va¹, and nₐ₁ each have the same definition as that for R, Va¹, and not in Formula (a1-1).

The acid dissociable group represented by General Formula (a1-r2-1), (a1-r2-3), (a1-r2-4), or (a1-r-4) is as described above. Among these, it is preferable to select those in which the acid dissociable group is a cyclic group because the reactivity is enhanced for EB or EUV, which is suitable.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (10)% by mole) of all constitutional units constituting the component (A1).

In a case where the proportion of the constitutional unit (a1) is set to be greater than or equal to the lower limits of the above-described preferable ranges, high sensitivity and etching resistance are likely to be achieved, and the roughness and the degree of light exposure margin ae likely to be improved. Further, in a case where the proportion of the constitutional unit (a1) is less than or equal to the upper limits of the above-described preferable ranges, the constitutional unit (a1) and other constitutional units can be balanced, and various lithography characteristics are enhanced.

In regard to constitutional unit (a10):

The constitutional unit (a10) is a constitutional unit represented by General Formula (a10-1) (here, a constitutional unit corresponding to the constitutional unit (a1) is excluded).

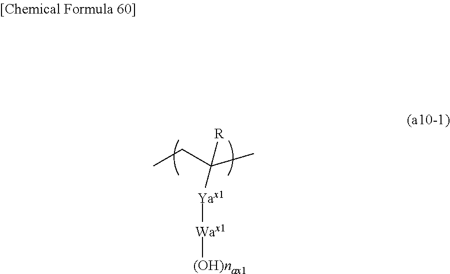

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent, nₐₓ₁ represents an integer of 1 or greater].

In Formula (a10-1), R has the same definition as that for R in General Formula (a1-1).

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Divalent Hydrocarbon Group which May have Substituent:

The divalent hydrocarbon group which may have a substituent may be an aliphatic hydrocarbon group or an aromatic hydrocarbon group.

### Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, mor preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₃—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —H(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atom have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group), a group in which two hydrogen atoms have been removed from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom

Examples of the divalent linking group having a heteroatom include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group or an acyl group), —S—, —S(═O)₂—, —S(═O)₂—O—, and a group represented by General Formula —Y²¹—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—[in the formulae, Y²¹ and Y²² each independently represent a divalent hydrocarbon group which may have a substituent, O represents an oxygen atom, and m″ represents an integer of 1 to 3].

In a case where the above-described divalent linking group having a heteroatom is —C(═O)—NH—, —C(═O)—NH—C(═O)—, —NH—, or —NH—C(═NH)—. H may be substituted with a substituent such as an alkyl group and an acyl group. The substituent (alkyl group, acyl group, and the like) preferably has 1 to 10 carbon atoms, more preferably has 1 to 8 carbon atoms, and particularly preferably has 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²², or —Y²¹—S(═O)₂—O—Y²²—, Y²¹ and Y²² each independently represents a divalent hydrocarbon group which may have a substituent. Examples of the divalent hydrocarbon group include the same groups as described above.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable. The alkyl group in the alkylmethylene group is preferably a linear alkyl group having 1 to 5 carbon atoms, more preferably a linear alkyl group having 1 to 3 carbon atoms, and most preferably a methyl group, in the group represented by Formula —[Y²¹—C(═O)—O]_{m″}—Y²², m″ represents an integer of 1 to 3, preferably an integer of 1 or 2, and more preferably 1. That is, a group represented by Formula —Y²¹—C(═O)—O—Y²²— is particularly preferable as the group represented by Formula —[Y²¹—C(═O)_{m″}—Y²²—. Among these, a group represented by Formula —(CH₂)_{a′}—C(═O)—O—(CH₂)_{b′}-2 is preferable, ln the formula, a′ represents an integer of 1 to 10, preferably an integer of 1 to 8, more preferably an integer of 1 to 5, still more preferably 1 or 2, and most preferably 1, b′ represents an integer of 1 to 10, preferably an integer of 1 to 8, more preferably an integer of 1 to 5, still more preferably 1 or 2, and most preferably 1.

Ya^{x1} represent, preferably a single bond, an ester bond (—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene, and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1 hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same groups as those for the substituent of the cyclic alicyclic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{e} represents a hydrogen atom, a methyl group, or a trifluoromethyl group.

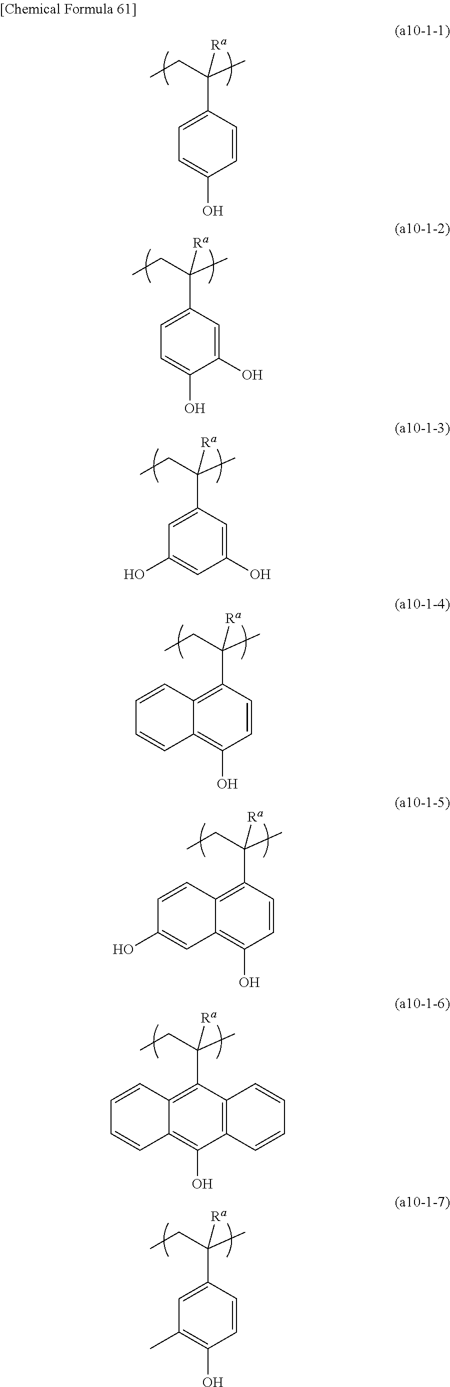

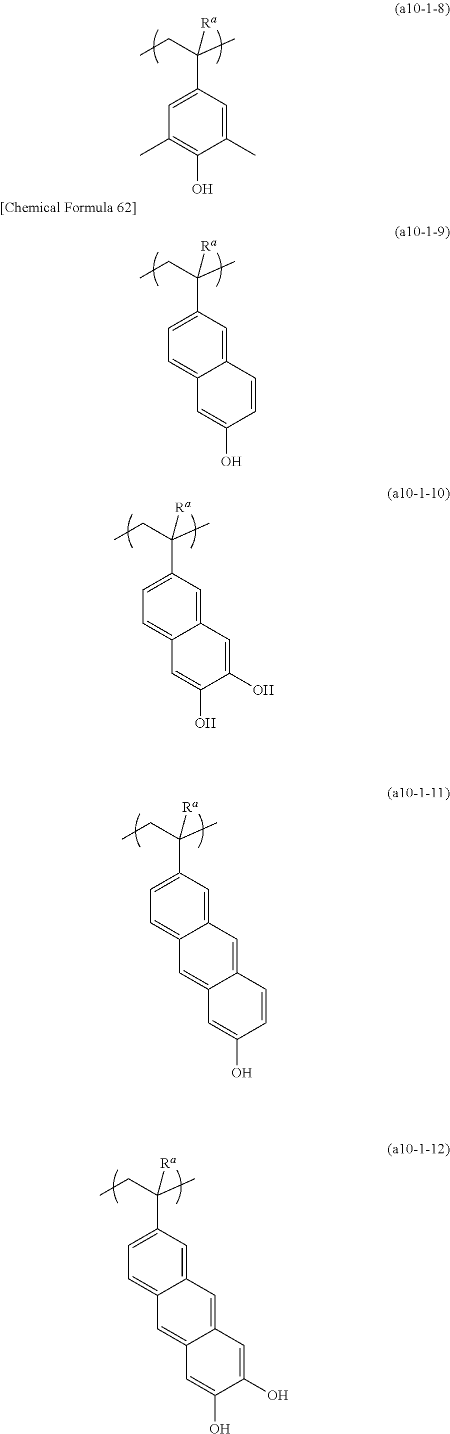

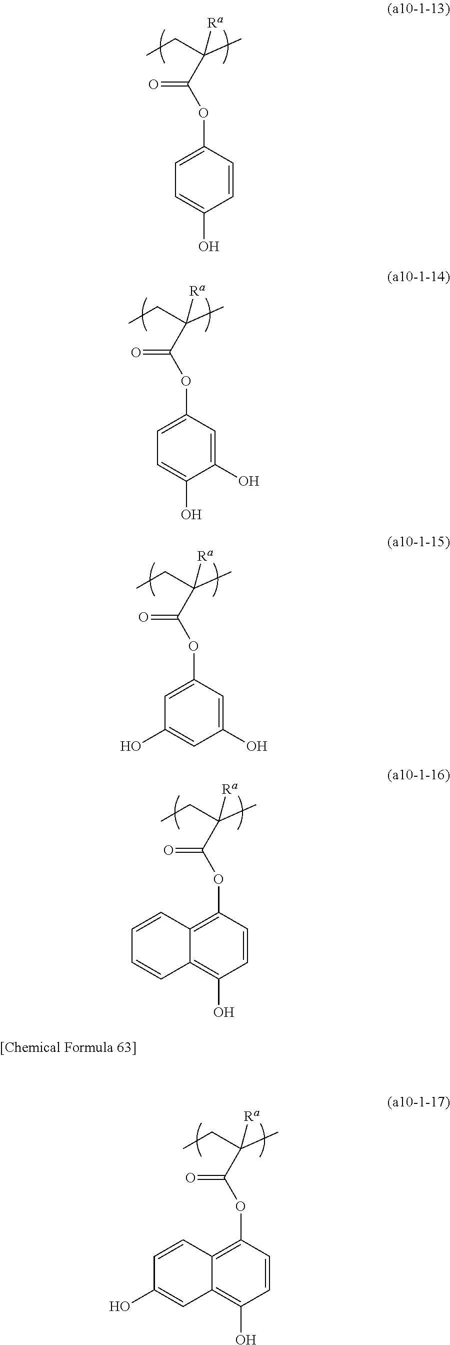

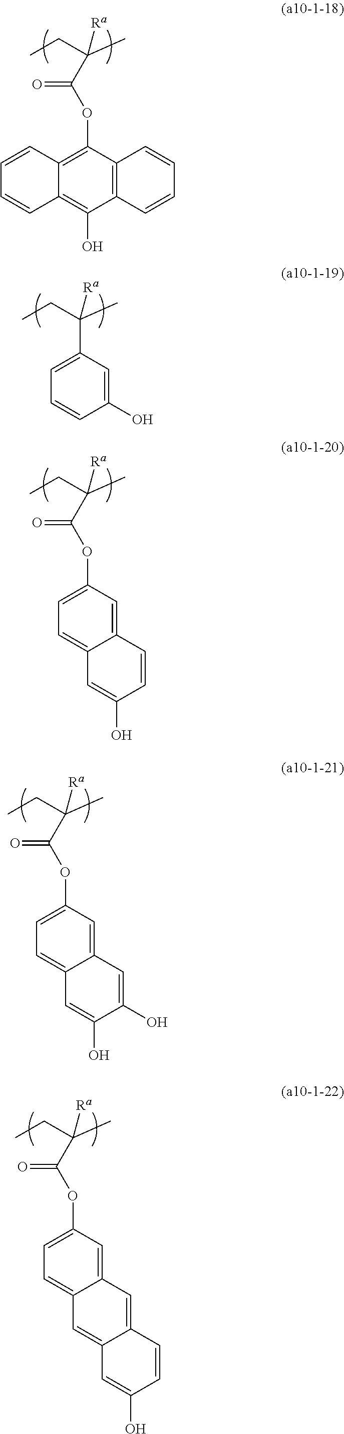

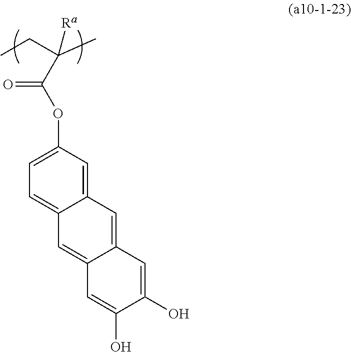

The constitutional unit (a10) that can be included in the component (A1) may be used alone or two or more kinds thereof.

The component (A1 may or may not have the constitutional unit a10), but it is preventable that the component (A1) has the constitutional unit (a10).

In a case where the component (A1) has the constitutional unit (a10), the proportion of the constitutional unit (a10) in the component (A1) is preferably in a range of 1% to 60% by mole, more preferably in a range of 15% to 50% by mole, and still more preferably in a range of 20% to 40% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the upper limits, the constitutional unit (a10) and other constitutional units are likely to be balanced.

In regard to constitutional unit (a5):

In the present embodiment, the constitutional unit (a5) is a constitutional unit which generates an acid upon light exposure, and a known constitutional unit can be used. In the constitutional unit (a5), those corresponding to the constitutional unit a0) are excluded. In a case where the component (A1) has the constitutional unit (a5), the acid generated upon light exposure is likely to be uniformly distributed in the resist film.

Suitable examples of the constitutional unit (a5) include a constitutional unit represented by General Formula (a5-1).

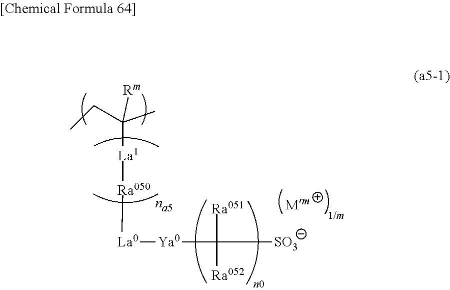

[In the formula, R^{m} represents an alkyl group having 1 to 5 carbon atoms, a halogenated alkyl group having 1 to 5 carbon atom, a halogen atom, or a hydrogen atom. La¹ represents a divalent linking group or a single bond. Ra⁰⁵⁰ represents a divalent hydrocarbon group which may have a substituent, nₐ₅ represents an integer of 0 to 2. La⁰ represents a divalent linking group. Ya⁰ represents a divalent linking group which may have a heteroatom or a single bond. Ra⁰⁵¹ and Ra⁰⁵² each independently represent a hydrogen atom, a fluorine atom, or a fluorinated alkyl group, n0 represents an integer of 1 to 4, m represents an integer of 1 or greater, and M′^{m+} represents an m-valent onium cation].

### {Anion Moiety}

In Formula (a5-1). R^{m} represents an alkyl group having 1 to 5 carbon atoms, a halogenated alkyl group having 1 to 5 carbon atoms, a halogen atom, or a hydrogen atom.

The alkyl group having 1 to 5 carbon atoms as R^{m} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms.

Examples of the halogen atom include a fluorine atom, a chlorine atom, a bromine atom, and an iodine atom. A fluorine atom is particularly preferable as the halogen atom in the halogenated alkyl group.

R^{m} represents preferably a hydrogen atom, an alkyl group having 1 to 5 carbon atoms, or a fluorinated alkyl group having 1 to 5 carbon atoms and, from the viewpoint of industrial availability, most preferably a hydrogen atom or a methyl group.

In Formula (a5-1), 1 represents a divalent linking group or a single bond.

The divalent linking group as La¹ is not particularly limited, and suitable examples thereof include a divalent hydrocarbon group which may have a substituent and a divalent linking group having a heteroatom, each of which has the same definition as the divalent hydrocarbon group which may have a substituent and the divalent linking group having a heteroatom, described as the divalent linking group represented by Ya^{x1}.

Among these, it is preferable that La¹ represents an ester bond [—C(═O)—O— or —O—C(═O)—], an ether bond (—O—), a linear or branched alkylene group, an aromatic hydrocarbon group, or a combination thereof, or a single bond. Among these. La¹ represents more preferably an ester bond [—C(═O)—O— or —O—C(═O)—] or a single bond and still more preferably an ester bond [—C(═O)—O— or —O—C(═O—], in Formula (a5-1), Ra⁰⁵⁰ represents a divalent hydrocarbon group which may have a substituent.

The divalent hydrocarbon group as Ra⁰⁵⁰ may be an aliphatic hydrocarbon group or an aromatic hydrocarbon group.

### Aliphatic Hydrocarbon Group as Ra⁰⁵⁰

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, mor preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—), a tetramethylene group —(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₂CH₂—, and —C(CH₂CH₃)₂—CH₂—; alkyltrimethylene groups such as —CH—(CH₃)CH₂CH₂, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The above linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable. Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂— is preferable.

### Aromatic Hydrocarbon Group as Ra⁰⁵⁰

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to carbon atoms, still more preferably 6 to 15 carbon atoms, and particularly preferably 6 to 12 carbon atoms. Here, the number of carbon atoms in a substituent is not included in the number of carbon atoms. Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The above-described alkylene group bonded to the aryl group or heteroaryl group has preferably 1 to 4 carbon atoms, more preferably 1 or 2 carbon atoms, and particularly preferably 1 carbon atom.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and most preferably a methyl group, an ethyl group, a propyl group, an n-butyl group, or a tert-butyl group.

Examples of the alkoxy group, the halogen atom, and the halogenated alkyl group as the substituents include those described as the substituent that substitutes a hydrogen atom in the cyclic aliphatic hydrocarbon group.

nₐ₅ represents an integer of 0 to 2.

Among these, Ra⁰⁵⁰ represents preferably an aliphatic hydrocarbon group having a ring in the structure, more preferably a cyclic aliphatic hydrocarbon group which may have a substituent having a heteroatom in the ring structure, and still more preferably an alicyclic hydrocarbon group which may have a substituent, which is a polycyclic group or a monocyclic group.

Alternatively, among the examples, it is preferable that Ra⁰⁵⁰ represents an aromatic hydrocarbon group.

In a case where nₐ₅ represents 2, both two Ra⁰⁵⁰'s may represent an alicyclic hydrocarbon group which may have a substituent, both two Ra⁰⁵⁰'s may represent an aromatic hydrocarbon group, or both two Ra⁰⁵⁰'s may represent a combination of an alicyclic hydrocarbon group which may have a substituent and an aromatic hydrocarbon group.

In Formula (a5-1), La⁰ represents a divalent linking group.

Examples of the divalent linking group as La⁰ include a non-hydrocarbon-based oxygen atom-containing linking group such as an oxygen atom (ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(—═O)—O—); and combinations of the above-described non-hydrocarbon-based oxygen atom-containing linking groups with an alkylene group. Further, a sulfonyl group (—SO₂—) may be further linked to the combination.

Examples of such a divalent linking group include linking groups each represented by General Formulae (L-al-1) to (L-al-9) described above. Further, in General Formulae (L-al-1) to (L-al-9). V′¹⁰¹ in General Formulae (L-al-1) to (L-al-9) is bonded to Ra⁰⁵⁰ in Formula (a5-1) described above.

La⁰ represents preferably a divalent linking group having an ester bond or a divalent linking group having an ether bond, more preferably a linking group represented by any of Formulae (L-al-1) to (L-al-5), (L-al-8), and (L-al-9), and still more preferably a linking group represented by (L-al-3) or (L-al-8).

In Formula (a5-1), Ya⁰ represents a divalent linking group which may have a heteroatom or a single bond.

The divalent linking group as Ya⁰ is not particularly limited, and suitable examples thereof include a divalent hydrocarbon group which may have a substituent, and a divalent linking group having a heteroatom.

The divalent hydrocarbon group which may have a substituent and the divalent linking group having a heteroatom as Ya⁰ each have the sane definition as the divalent hydrocarbon group which may have a substituent and the divalent linking group having a heteroatom, described as the divalent linking group represented by Ya^{x1}.

Among the examples, Ya⁰ represents preferably a linear or branched alkylene group or a single bond and more preferably a single bond.

In Formula (a5-1). Ra⁰⁵¹ and Ra⁰⁵² each independently represent a hydrogen atom, a fluorine atom, or a fluorinated alkyl group.

The fluorinated alkyl groups as Ra⁰⁵¹ and Ra⁰⁵² are each preferably a linear or branched fluorinated alkyl group having 1 to 5 carbon atoms and more preferably a trifluoromethyl group.

In Formula (a5-1), it is preferable that at least one of Ra⁰⁵¹ and Ra⁰⁵² bonded to the carbon atom adjacent to SO₃⁻ represents a fluorine atom from the viewpoint of acid strength.

In Formula (a5-1), n0 represents an integer of 1 to 4 and preferably 1, 2, or 3.

### {Cation Moiety}

In Formula (a5-1). M′^{m+} represents an in-valent onium cation, m and M′^{m+} each have the same definition as that for in and M^{m+} in General Formula (a0-m).

Specific preferred examples of the constitutional unit (a5) are shown below.

In the formulae shown below, R^{α} represents a hydrogen atom, a methyl group, or a trifluoromethyl group, m and M′^{m+} each have the sane definition as that for m and M′^{m+} in General Formula (a5-1).

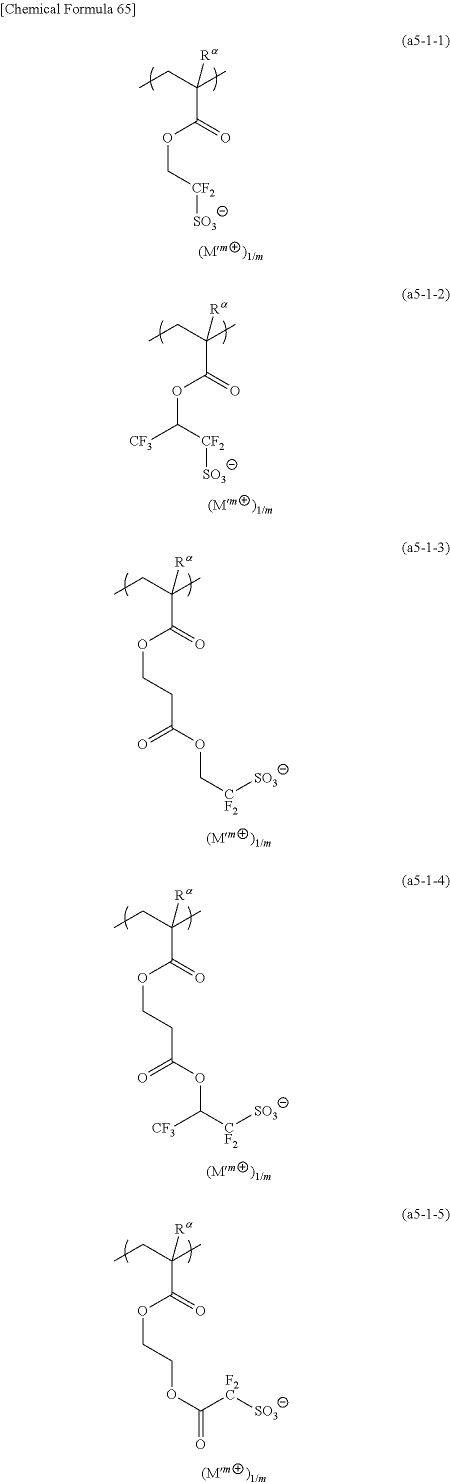

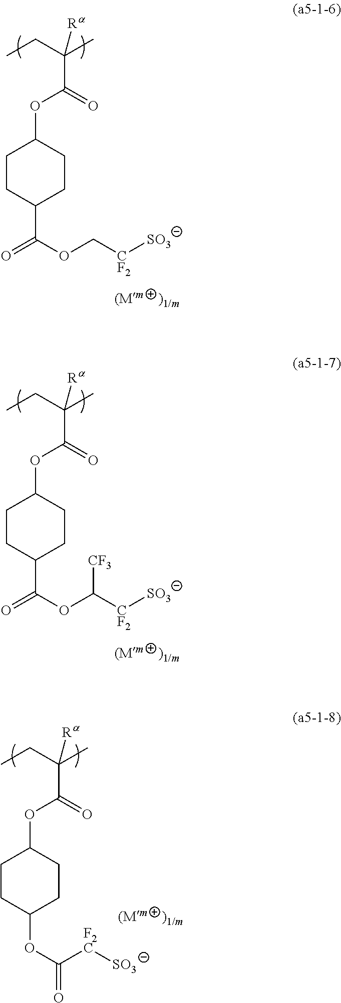

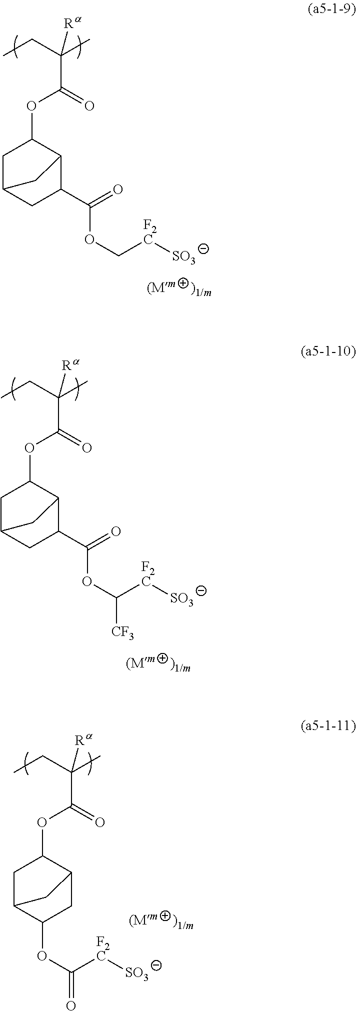

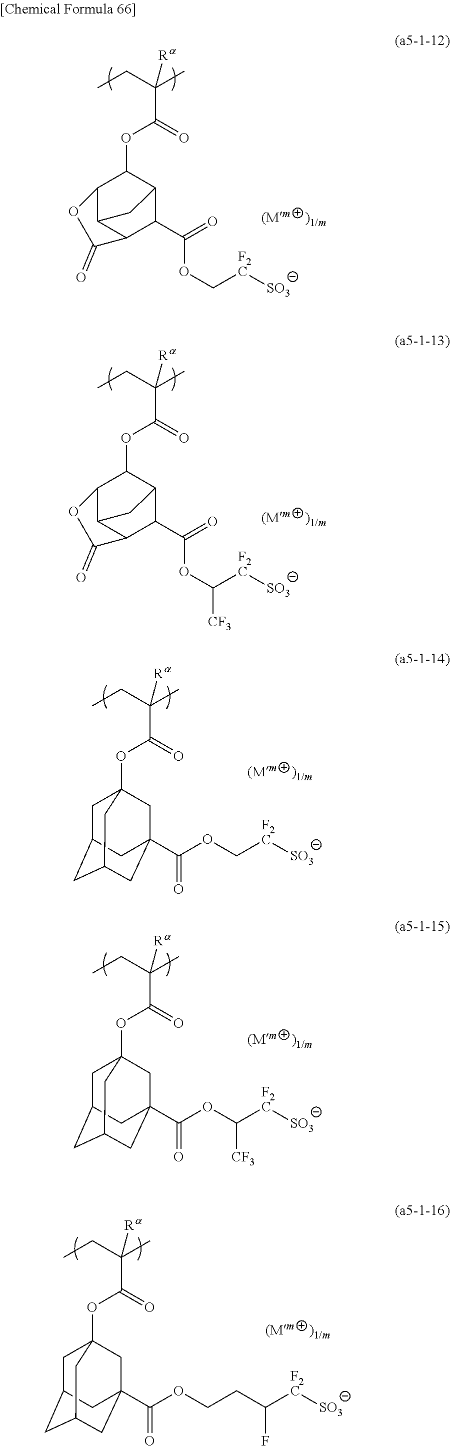

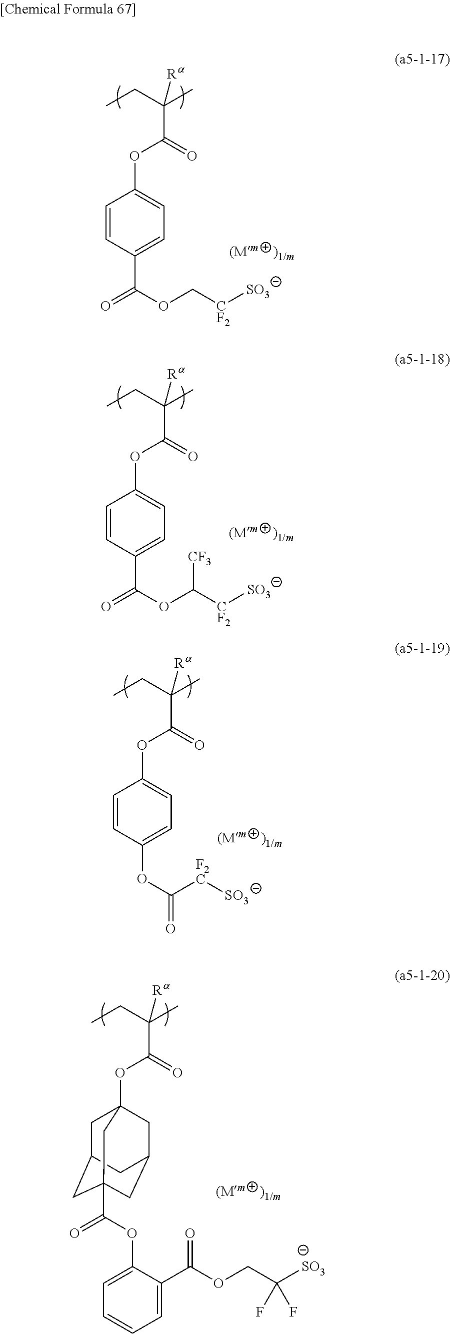

The constitutional unit (a5) of the component (A1) may be used alone or two or more kinds thereof.

In a case where the component (A1) has the constitutional unit (a5), the proportion of the constitutional unit (a5) in the component (A1) is preferably in a range of 5% to 25% by mole, more preferably in a range of 10% to 20% by mole, and still more preferably in a range of 15% to 20% by mole with respect to the total amount (100% by mole) of all constitutional units constituting the component (A1).

In a case where the proportion of the constitutional unit (a5) is equal to or greater than the lower limit of the above-described preferred range, it is easy to realize further improvement in sensitivity and reduction in roughness. Meanwhile, in a case where the proportion thereof is less than or equal to the upper limits of the above-described preferable ranges, the constitutional unit (a5) and other constitutional units are likely to be balanced.

Regarding constitutional unit (a6) having acid diffusion controllability:

The constitutional unit (a6) is a constitutional unit having acid diffusion controllability.

The component (A1) may or may not have the constitutional unit (a6). As the constitutional unit (a6), a known constitutional unit can be used. Examples of the constitutional unit (a6) include a constitutional unit having a structure described in the section of the component (D1) and the component (D2) below. Examples thereof include a constitutional unit having a structure represented by any of General Formulae (d1-1) to (d1-3).

The constitutional unit (a6) contained in the component (A1) may be used alone or two or more kinds thereof.

In a case where the component (A1) has the constitutional unit (a6), the proportion of the constitutional unit (a6) in the component (A1) is preferably in a range of 1% to 20% by mole, more preferably in a range of 2% to 15% by mole, and still more preferably in a range of 3% to 10% by mole with respect to the total amount (100% by mole) of all constitutional units constituting the component (A1).

In a case where the proportion of the constitutional unit (a6) is greater than or equal to the lower limits of the above-described preferable ranges, higher sensitivity is likely to be realized. Meanwhile, in a case where the proportion thereof is less than or equal to the tipper limits of the above-described preferable ranges, the constitutional unit (a5) and other constitutional units are likely to be balanced.

In regard to constitutional unit (a2):

The component (A1) may further have a constitutional unit (a2) (here, a constitutional unit corresponding to the constitutional unit (a1) is excluded) containing a lactone-containing cyclic group.

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are enhanced, for example, due to the effects of appropriately adjusting the acid diffusion length, increasing the adhesiveness of the resist film to the substrate, appropriately adjusting the solubility during the development, and the like.

The term “lactone-containing cyclic group” indicates a cyclic group that has a ring (lactone ring) containing —O—C(═O)—in the ring skeleton, in a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

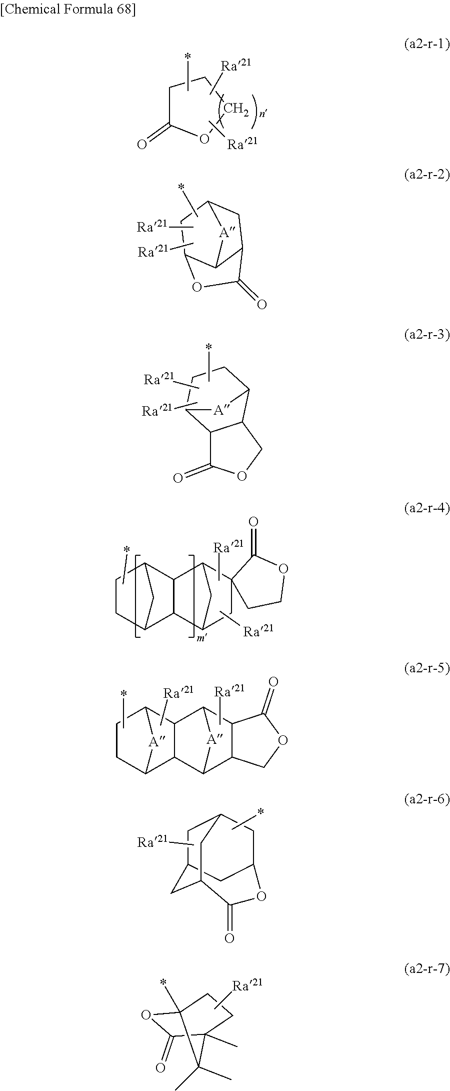

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group. A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter)].

In General Formulae (a2-r-1) to (a2-r-7), it is preferable that the alkyl group as Ra′²¹ is an alkyl group having 1 to 6 carbon atoms. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represent, a hydrogen atom, an alkyl group, or a lactone-containing cyclic group.

The alkyl group as R″ may be linear, branched, or cyclic and has preferably 1 to carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at (east one hydrogen atom in the alkyl group as Ra′²¹ has been substituted with a hydroxyl group.

Among the examples, it is preferable that each Ra′²¹ independently represent a hydrogen atom or a cyano group.

In General Formulae a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups having an oxygen atom or a sulfur atom include a group obtained by interposing —O— or —S—in the terminal of the alkylene group or between the carbon atoms of the alkylene group, and examples thereof include —O—CH₂—, —CH—CH, —S—CH₂ and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

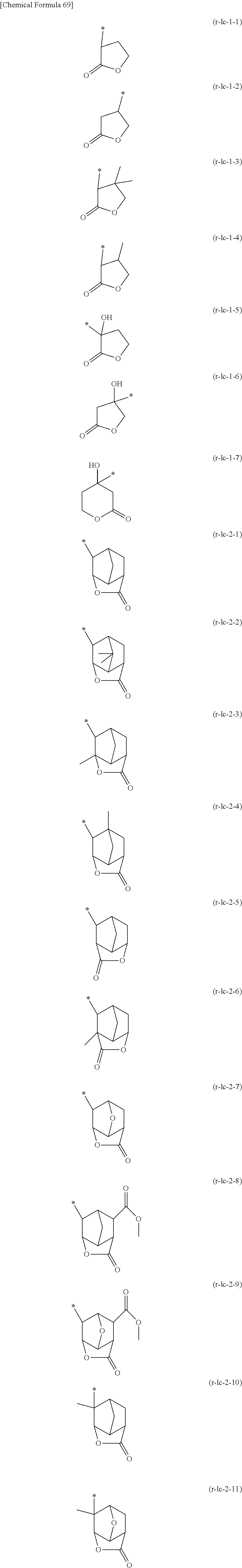

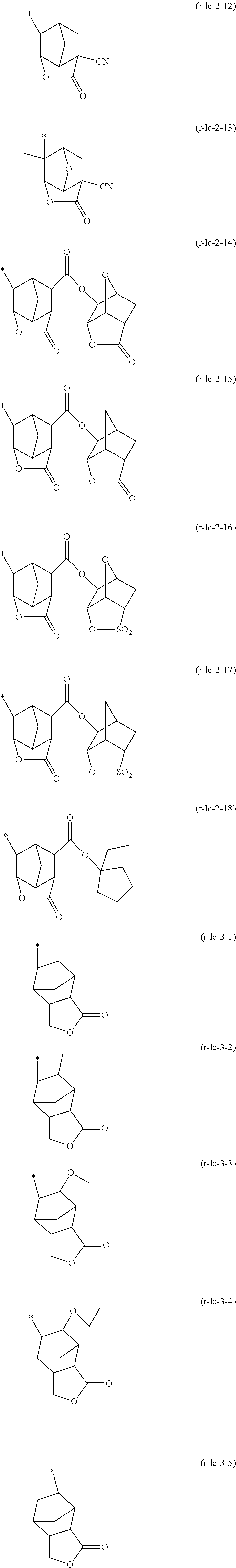

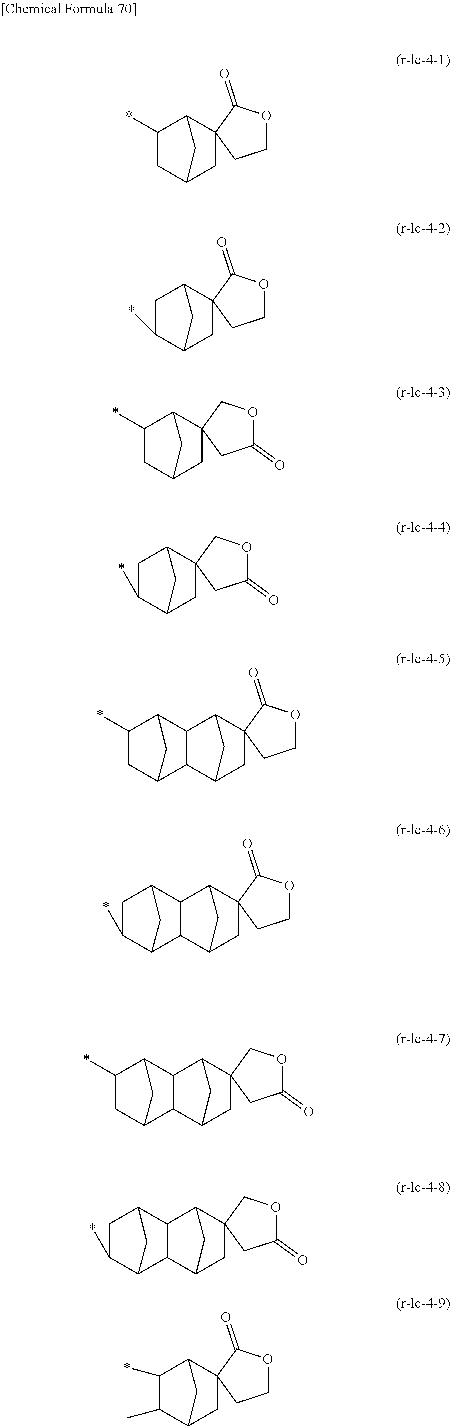

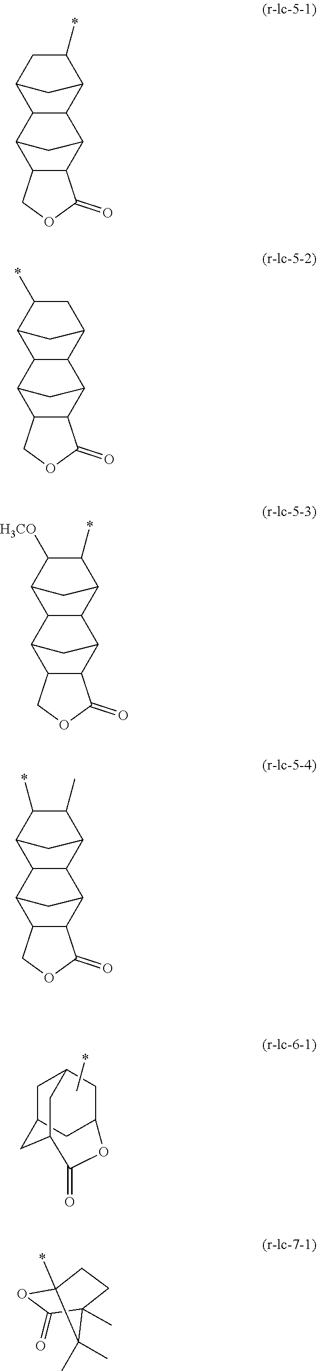

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

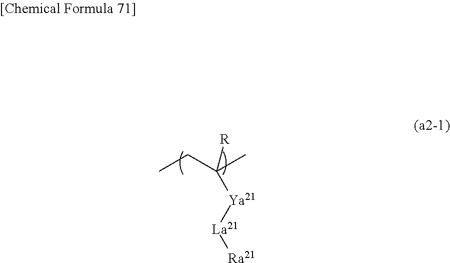

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group].

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom.

Examples of the divalent linking group as Ya²¹ include the same groups as those for the divalent linking group as Ya^{x1} in General Formula (a10-1).

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²³ include groups each represented by General Formulae (a2-r-1) to (a2-r-7).

The constitutional unit (a2) that can be included in the component (A1) may be used alone or two or more kinds thereof.

The component (A1) may or may not have the constitutional unit (a2).

In a case where the component (A1) has the constitutional unit (a2), the proportion of the constitutional unit (a2) is preferably in a range of 1% to 20% by mole, more preferably in a range of 2% to 15% by mole, and still more preferably in a range of 5% to 15% by mole with respect to the total amount (100% by mole) of all constitutional units constituting the component (A1).

In a case where the proportion of the constitutional unit (a2) is set to be greater than or equal to the above-described preferable lower limits, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and various lithography characteristics are enhanced.

In regard to constitutional unit (a8):

The constitutional unit (a8) is a constitutional unit derived from a compound represented by General Formula (a8-1).

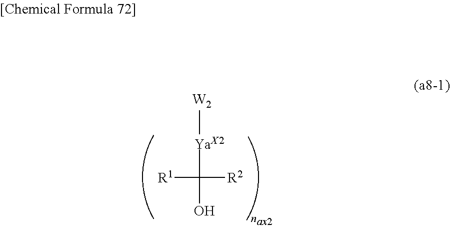

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom, or a hydrogen atom, R² and Ya^{x2} may be bonded to each other to form a ring structure, nₐₓ₂ represents an integer of 1 to 3].

The term “polymerizable group” in the polymerizable group-containing group as W² is a group that enables a compound having the polymerizable group to be polymerized by radical polymerization or the like, and refers to a group containing multiple bonds between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group having a heteroatom.

Suitable examples of the polymerizable group-containing group include a group represented by a chemical formula: C(R^{X11})(R^{X12})═C(R^{X13})-Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

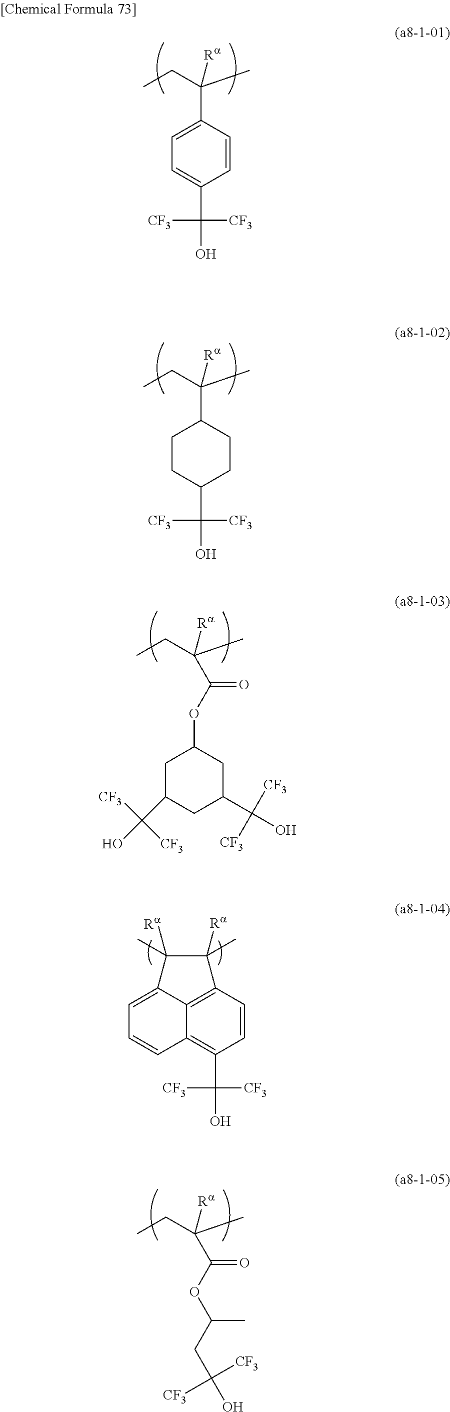

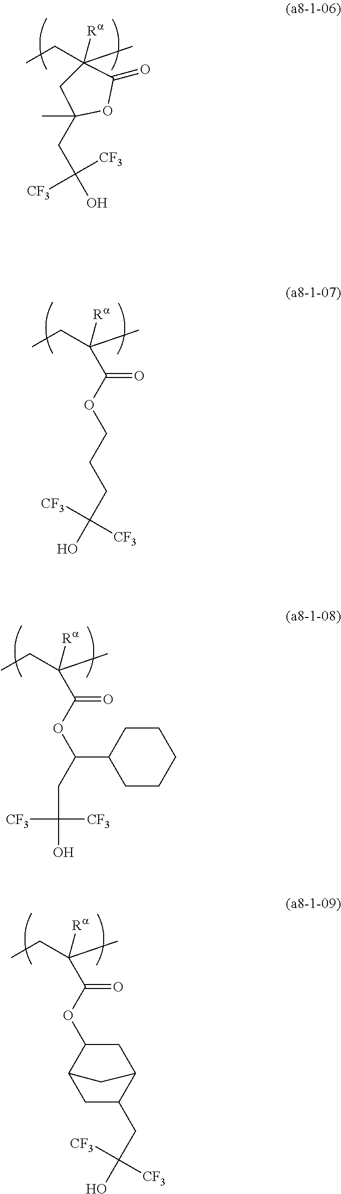

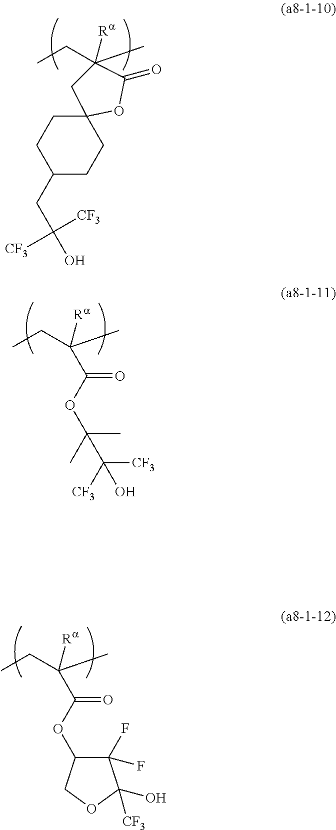

Among the examples, the constitutional unit (a8) is preferably at least one selected from the group consisting of constitutional units each represented by Chemical Formulae (a8-1-01) to (a8-1-04), (a8-1-(06), (a8-1-08), (a8-1-09), and (a8-1-10), and more preferably at least one selected from the group consisting of constitutional units each represented by Chemical Formulae (a8-1-01) to (a8-1-04) and (a8-1-09).

The constitutional unit (a8) that can be included in the component (A1) may be used alone or two or more kinds thereof.

The component (A1) may or may not have the constitutional unit (a8).

In a case where the component (A1) has the constitutional unit (a8), the proportion of the constitutional unit (a8) in the component (A1) is preferably 30% by mole or less, more preferably 0% to 20% by mole, and still more preferably 0% to 10% by mole with respect to the total amount (100% by mole) of all constitutional units constituting the component (A1).

The component (A1) contained in the resist composition may be used alone or in combination of two or more kinds thereof.

Among the examples, as the component (A1), a polymer compound having a repeating structure of the constitutional unit (00), the constitutional unit (a10), and the constitutional unit (a1) is preferable, and a polymer compound formed of only a repeating structure of the constitutional unit (a0), the constitutional unit (a10), and the constitutional unit (a1) is more preferable.

The proportion of the constitutional unit (a0) in the polymer compound having a repeating structure of the constitutional unit (a1), the constitutional unit (a10), and the constitutional unit (a1) is preferably in a range of 2% to 30% by mole, more preferably in a range of 5% to 25% by mole, and still more preferably in a range of 5% to 20% by mole with respect to the total amount (100% by mole) of all constitutional units constituting the polymer compound.

Further, the proportion of the constitutional unit (a10) in the polymer compound is preferably in a range of 10% to 60% by mole, more preferably in a range of 15% to 50% by mole, and still more preferably in a range of 20% to 40% by mole with respect to the total amount (100% by mole) of all constitutional units constituting the polymer compound.

Further, the proportion of the constitutional unit (a1) in the polymer compound is preferably in a range of 10% to 80% by mole, more preferably in a range of 15% to 75% by mole, and still more preferably in a range of 20% to 70% by mole with respect to the total amount (100% by mole) of all constitutional units constituting the polymer compound.

Here, the total proportion of the constitutional unit (a0), the constitutional unit (a10), and the constitutional unit (a1) does not exceed 100% by mole.

Alternatively, among the examples, as the component (A1), a polymer compound having a repeating structure of the constitutional unit (a0), the constitutional unit (a10), the constitutional unit (a2), and the constitutional unit (a1) is preferable, and a polymer compound formed of only a repeating structure of the constitutional unit (a0), the constitutional unit (a10), the constitutional unit (a2), and the constitutional unit (a1) is more preferable.

The proportion of the constitutional unit (a0) in the polymer compound having a repeating structure of the constitutional unit (a0), the constitutional unit (a10), the constitutional unit (a2), and the constitutional unit (a1) is preferably in a range of 2% to 30% by mole, more preferably in a range of 5% to 25% by mole, and still more preferably in a range of % to 20% by mole with respect to the total amount (100% by mole) of all constitutional units constituting the polymer compound.

Further, the proportion of the constitutional unit (a10) in the polymer compound is preferably in a range of 10% to 60% by mole, more preferably in a range of 15% to 50% by mole, and still more preferably in a range of 20% to 40% by mole with respect to the total amount (100% by mole) of all constitutional units constituting the polymer compound.

Further, the proportion of the constitutional unit (a2) in the polymer compound is preferably in a range of 1% to 20% by mole, more preferably in a range of 2% to 15% by mole, and still more preferably in a range of 5% to 15% by mole with respect to the total amount (100% by mole) of all constitutional units constituting the component (A1).

Further, the proportion of the constitutional unit (a1) in the polymer compound is preferably in a range of 10% to 80% by mole, more preferably in a range of 15% to 75% by mole, and still more preferably in a range of 20% to 70% by mole with respect to the total amount (100% by mole) of all constitutional units constituting the polymer compound.

However, the total of the ratios of the constitutional unit (a0), the constitutional unit (a10), the constitutional unit (a2), and the constitutional unit (a1) is not mote than 100 mol %.

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a0) is derived and, as necessary, a monomer from which a constitutional unit (for example, the constitutional unit (a10)) other than the constitutional unit (a0) is derived, adding such a radical polymerization initiator thereto to carry out polymerization, and carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 5000, more preferably in a range of 2000 to 30000, and still more preferably in a range of 3000 to 2000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are satisfactory.

Further, the dispersity (Mw/Mn) of the component (A1, is not particularly limited, but is preferably in a range of 1.0 to 4.0, more preferably in a range of 1.0 to 3.0, and particularly preferably in a range of 1.0 to 2.0. Further. Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist, pattern having excellent various lithography characteristics such as high sensitivity, high resolution, and improved roughness is likely to be formed.

In the resist composition of the present embodiment, the content of the component (A) may be adjusted according to the thickness and the like of the resist film intended to be formed.

### <Other Components>

The resist composition according to the present embodiment may further contain other components in addition to the component (A) described above. Examples of the other components include a component (B), a component (D)), a component (E), a component (F), and a component (S), which are described below.

### <<Component (B): Acid Generator Component>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as indonium salts and sulfonium salts; oxime sulfonate-based acid generators diazomethane-based acid generators such as bisalkyl or bisaryl sulfonyl diazomethanes and poly(his-sulfonyl)diazomethanes; nitrobenzyl sulfonate-based acid generators, iminosulfonate-based acid generators, and disulfone-based acid generators.

The component (B) may be contained in the form of a compound, in the form in which the component (B) is incorporated into the component (A1) as the constitutional unit (a5) described above, or in both forms.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

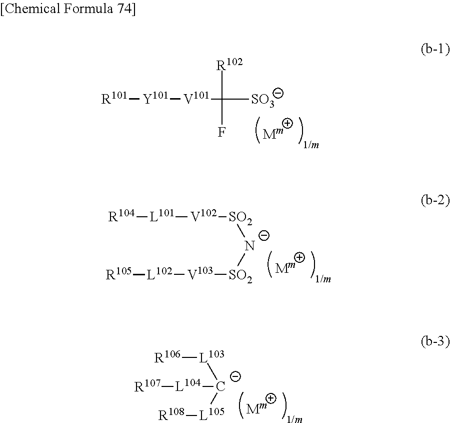

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. Here, Y¹⁰¹ and V¹⁰¹ do not represent a single bond at the same time. L¹⁰¹ and L¹⁰² each independently represent a single bond or an oxygen atom. L¹⁰³ to L¹⁰⁵ each independently represent a single bond, —CO—, or —SO₂—, m represents an integer of 1 or greater, and M′^{m+} represents an m-valent onium cation].

### {Anion Moiety}

Anions in Component (b-1)

In Formula (b-1). R¹⁰¹ represents a cyclic group which may have a substituent, a chain-like alkyl group which may have a substituent, or a chain-like alkenyl group which may have a substituent.

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring-based polycyclic skeleton such as adamantane, norbornane, isobornane, tricyclodecane, or tetracyclododecane, and a polycycloalkane having a condensed ring-based polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group (—(CH₂)₃—], a tetramethylene group [—(CH)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₂CH₂—, and —C(CH₂CH₃)₂—CH₂—; alkyltrimethylene groups such as —CH—(CH₃)CH₂CH₂, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site with respect to Y¹⁰¹ in Formula (b-1).

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include those obtained by condensing one or mor aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

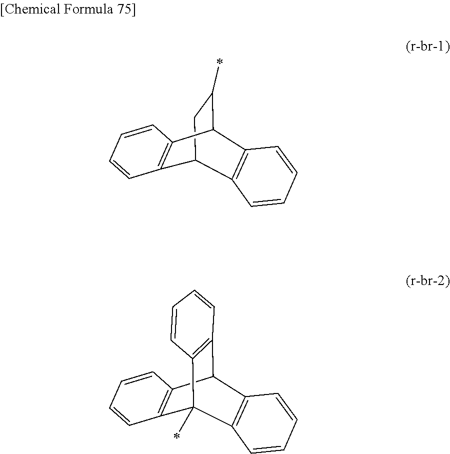

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include the same groups as those for the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom ha; been removed from a polycycloalkane such as adamantane, norborane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-like alkyl group which may have substituent:

The chain-like alkyl group as R¹⁰¹ may be linear or branched.

The number of carbon atoms in the linear alkyl group is preferably in a range of 1 to 20, more preferably in a range of 1 to 15, and most preferably in a range of 1 to 10.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a -methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

In Formula (b-1). Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-al-1) to (y-al-7). In General Formulae (y-al-1) to (y-al-7), V′¹⁰¹ in General Formulae (y-al-1) to (y-al-7) is bonded to R¹⁰¹ in Formula (b-1).

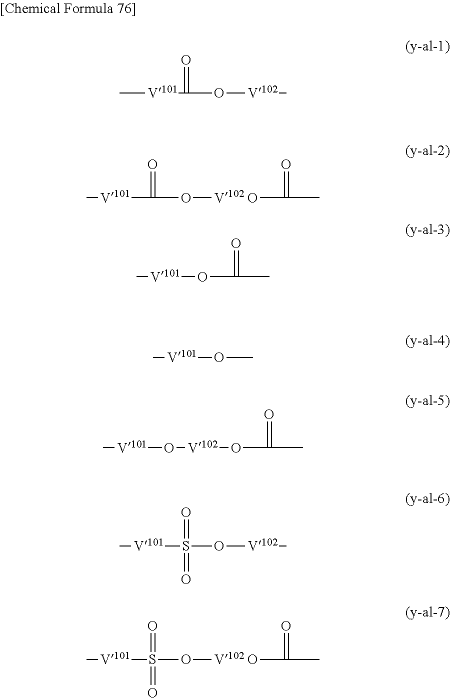

[In the formulae. V′¹⁰¹ represents a single bond or an alkylene group having 1 to carbon atoms, and V′¹⁰² represents a divalent saturated hydrocarbon group having 1 to carbon atoms].

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂—, or —CH(CH₂CH₂CH₂—; a trimethylene group (n-propylene group) [—CH₂CH₂CH₂—]; an alkyltrimethylene groups such as —CH(CH₃)CH₂CH₂— or —CH₂CH(CH₃)CH₂—; a tetramethylene groups such as [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

In Formula (b-1). V¹⁰¹ represents a single bond, an alkylene group, or a fluorinated alkylene group. Among these, it is preferable that V¹⁰¹ represent, a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific examples of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

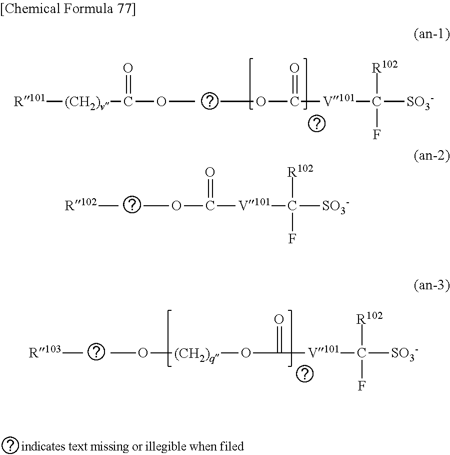

[In the formulae, R″¹⁰¹ represents an aliphatic cyclic group which may have a substituent, monovalent heterocyclic groups each represented by Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —S(2-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1].

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the groups described as the aromatic hydrocarbon group in the cyclic hydrocarbon group as R¹⁰¹ in Formula (b-1) are preferable. Examples of the substituent include the same substituents as those which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2). V¹⁰² and V¹⁰³ each independently represent a single bond, an alkylene group, or a fluorinated alkylene group, and examples of each of the groups include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L¹⁰³ to L¹⁰⁵ each independently represent a single bond, —CO—, or —SO₂—.

Among these, as the anion moiety of the component (B), an anion of the component (b-1) is preferable, and an anion represented by Formula (an-1) is more preferable.

### {Cation Moiety}

In Formulae (b-1), (b-2), and (b-3), M′^{m+} represents an m-valent onium cation, m and M′^{m+} each have the same definition as that for in and M^{m+} in General Formula (a0-m).

Among them, a sulfonium cation and an iodonium cation are preferable, m represents an integer of 1 or greater.

Among the examples, as the cation moiety of the component (B), a sulfonium cation is preferable, a cation represented by any of Formulae (ca-1) to (ca-3) is more preferable, a cation represented by Formula (ca-1) is still more preferable, and a cation represented by any of Formulae (ca-1-1) to (ca-1-85) is particularly preferable.

In the resist composition according to the present embodiment, the component (B) may be used alone or in combination of two or more kinds thereof.

The content of the component (B) in the resist composition is preferably less than 50 parts by mass, more preferably in a range of 0 to 40 parts by mass, still more preferably in a range of 0 to 30 parts by mass, and still more preferably in a range of 0 to parts by mass with respect to 10 parts by mass of the component (A1).

In a case where the content of the component (B) is set to be in the above-described preferable ranges, pattern formation can be sufficiently carried out. Further, it is preferable that each component of the resist composition is dissolved in an organic solvent from the viewpoint that, a uniform solution is easily obtained and the storage stability of the resist composition is enhanced.

### <<Base Component (D)>>

The resist composition of the present embodiment may further contain, in addition to the component (A), a base component (component (D)) that traps an acid generated upon light exposure (that is, controls diffusion of an acid). The component (D) acts as a quencher (an acid diffusion control agent) which traps the acid generated in the resist composition upon light exposure.

Examples of the component (D) include a photodecomposable base (D) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (component (D1)) is preferable from the viewpoint that all the characteristics of high sensitivity, roughness reduction, and suppression of occurrence of coating defects are likely to be enhanced. The compound described as the component (D1) below may be used as the acid generator component (component (B)) depending on the combination with other compounds.

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

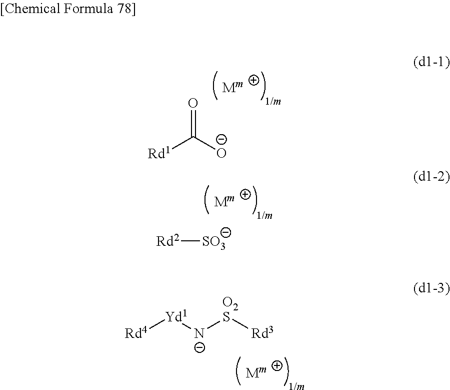

[In the formulae. Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d-2) has no fluorine atom bonded thereto. Yd¹ represents a single bond or a divalent linking group, m represents an integer of 1 or greater, and M^{m+}'s each independently represents an in-valent organic cation].

{Component (d1-1}

### Anion Moiety

In Formula (d1-1). Rd¹ represents a cyclic group which may have a substituent, a chain-like alkyl group which may have a substituent, or a chain-like alkenyl group which may have a substituent, and examples of each of the cyclic groups include the same groups as those for R′²⁰¹.

Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. Further, in a case where the aromatic hydrocarbon group, the aliphatic cyclic group, or the chain-like alkyl group as Rd¹ contains a linking group represented by any of General Formulae (y-al-1) to (y-al-7) as a substituent, V′¹⁰¹ in General Formula (y-al-1) to (y-al-7) is bonded to the carbon atom constituting the aromatic hydrocarbon group, the aliphatic cyclic group, or the chain-like alkyl group as Rd¹ in General Formula (d3-1), in Formulae (y-al-1) to (y-al-7).

Suitable examples of the aromatic hydrocarbon group include a phenyl group, a naphthyl group, and a polycyclic structure having a bicyclooctane skeleton ta polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

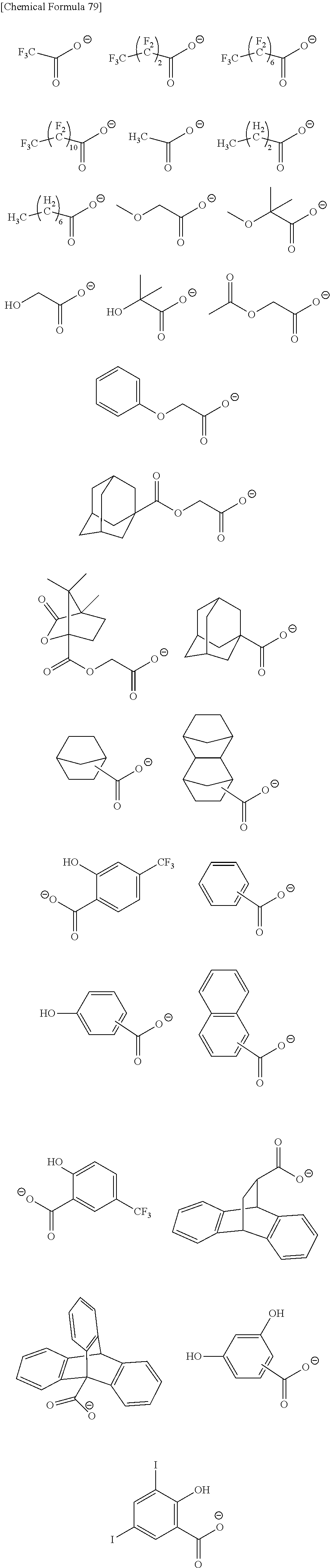

### Cation Moiety

In Formula (d1-1). M^{m+} represents an n-valent organic cation.

Suitable examples of the organic cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-85) are still more preferable. The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent, to the S atom in Rd² has no fluorine atom bonded thereto (the carbon atom is not substituted with fluorine). As a result, the anion of the component (d1-2) becomes an appropriately weak acid anion, thereby improving the quenching ability of the component (D).

It is preferable that Rd² represents a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclodecane, tetracyclododecane, or the like (a group which may have a substituent); and a group in which one or more hydrogen atoms have been removed from camphor or the like are more preferable.

The hydrocarbon group as Rd² may have a substituent, and example, of the substituent include the same substituents as those that the hydrocarbon group (an aromatic hydrocarbon group, an aliphatic cyclic group, or a chain-like alkyl group) as Rd¹ in Formula (d1-1) may have.

Specific preferred examples of the anion moiety in the component (d1-2) are described below.

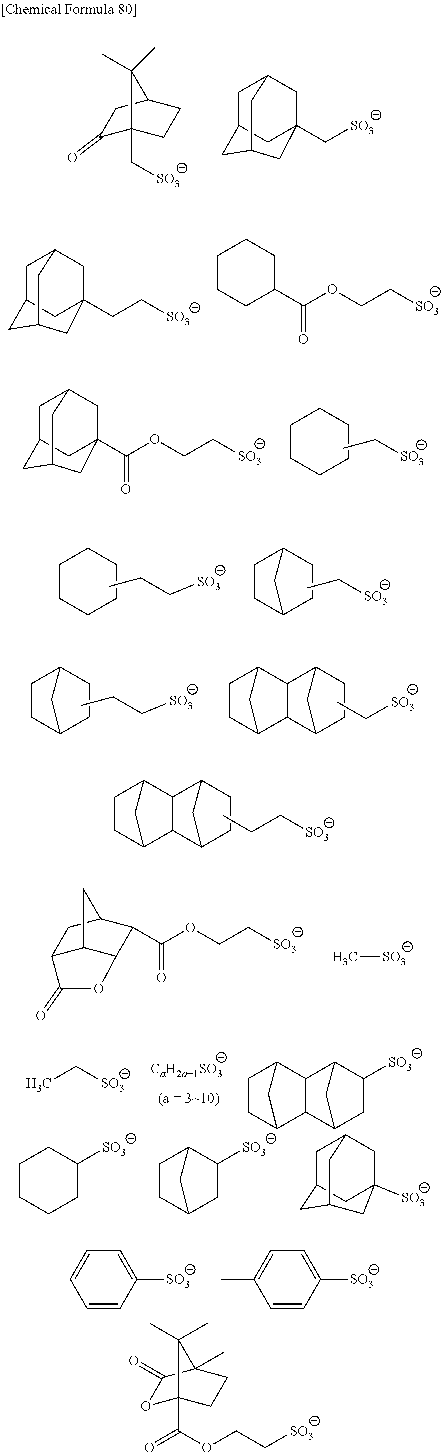

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

{Component (d11-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3). Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may further contain an alkyl group having 1 to 5 carbon atoms or a halogenated alkyl group having 1 to 5 carbon atoms as a substituent.

Examples of the cyclic group as Rd⁴ include the same groups as those for the cyclic group as R′⁴⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3). Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

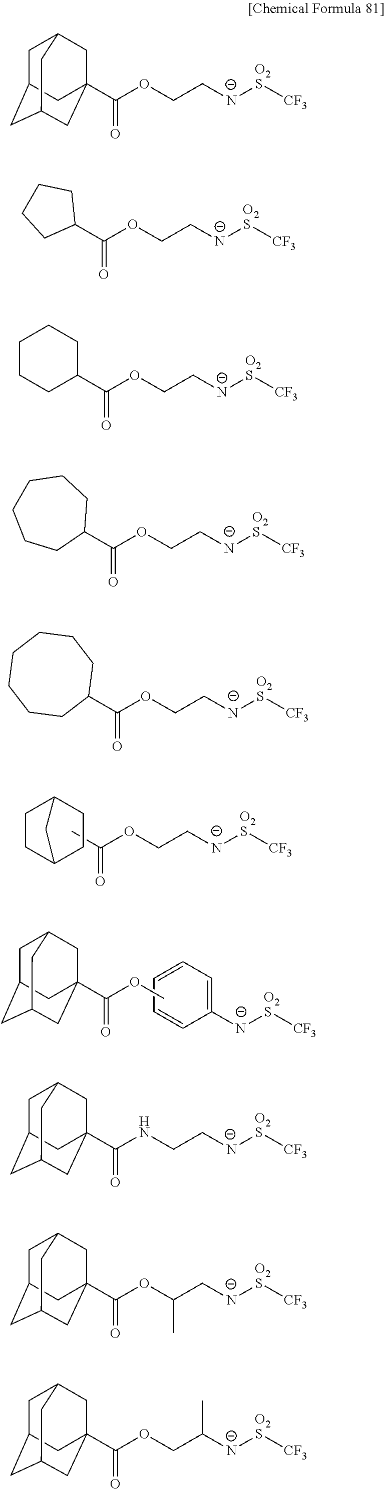

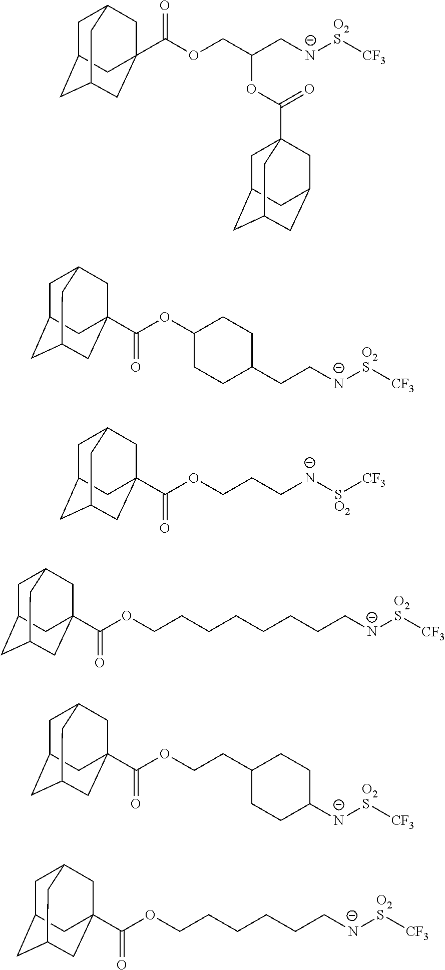 [SMILES: O=C(OCC(C[N-]S(=O)(=O)C(F)(F)F)OC(=O)C12CC3CC(CC(C3)C1)C2)C12CC3CC(CC(C3)C1)C2 ; O=C(OC1CCC(CC[N-]S(=O)(=O)C(F)(F)F)CC1)C12CC3CC(CC(C3)C1)C2 ; O=C(OCCC[N-]S(=O)(=O)C(F)(F)F)C12CC3CC(CC(C3)C1)C2 ; O=C(OCCCCCCCC[N-]S(=O)(=O)C(F)(F)F)C12CC3CC(CC(C3)C1)C2 ; O=C(OCCC1CCC([N-]S(=O)(=O)C(F)(F)F)CC1)C12CC3CC(CC(C3)C1)C2 ; O=C(OCCCCCC[N-]S(=O)(=O)C(F)(F)F)C12CC3CC(CC(C3)C1)C2]

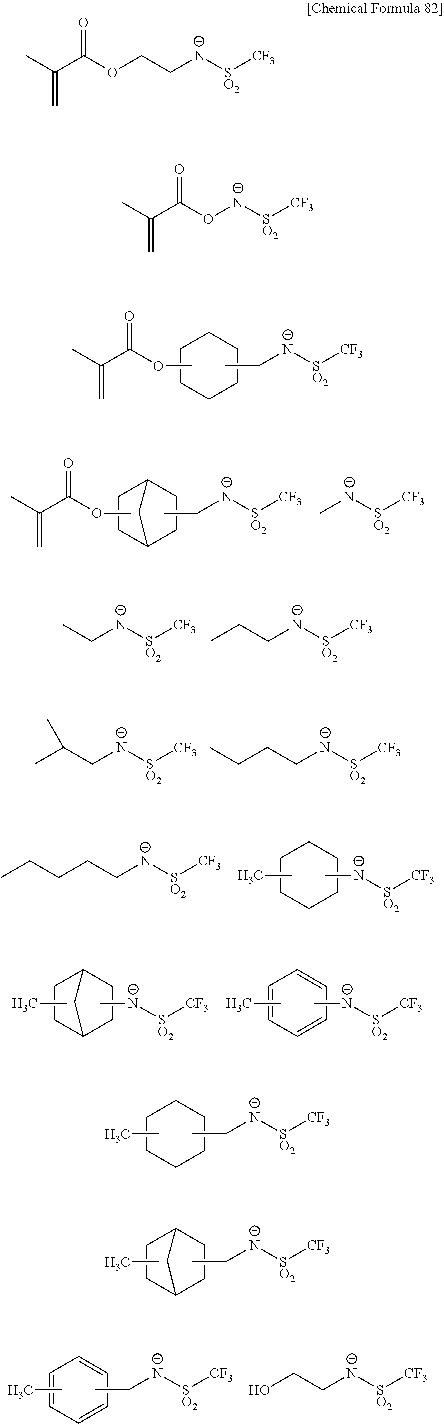

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only any one of the above-described components (d1-1) to (d1-3) or a combination of two or more kinds thereof may be used.

In a case where the resist composition contains the component (D1), the content of the component (D1) in the resist composition is preferably in a range of 0.5 to 20 pans by mass, more preferably in a range of 1 to 15 parts by mass, and still more preferably in a range of 2 to 10 parts by mass with respect to 100 parts by mass of the component (A1).

In a case where the content of the component (D1) is greater than or equal to the lower limits of the above-described preferable ranges, satisfactory lithography characteristics and a satisfactory resist pattern shape are likely to be obtained. On the contrary, in a case where the content is less than or equal to the upper limits of the above-described ranges, the sensitivity can be satisfactorily maintained and the throughput is also excellent.

Method of producing component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as it acts as an acid diffusion control agent and does not correspond to the component (D1), and any known compound may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine: trialkylamines such as trimethylamine, triethylamine, tri-n-propylamine, tri-n-butylamine, tri-n-pentylamine, tri-n-hexylamine, tri-n-heptylamine, tri-n-ocylanine, tri-n-nonylamine, tri-n-decylanine, and tri-n-dodecylamine; and alkylatcoholantines such as diethanoliamine, triethanolamine, diisopropanolainne, triisopropanolanine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

It is preferable that the aliphatic polycyclic amine has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxycthoxy)ethyl}amine, tris{2-(1-ethoxyethoxy)ethyl}amine, tris{2-(1-ethoxypropoxy)ethyl}amine, tris[2-(2-(2-hydroxyethoxy)ethoxy)ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline. N-tert-butoxycarbonylpyrrolidin, and 2,6-di-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A1). In a case where the content thereof is set to be in the above-described range, the resist pattern shape, the post-exposure temporal stability, and the like are improved.

<<At Least One Compound (E) Selected from Group Consisting of Organic Carboxylic Acids, Phosphorus Oxo Acids. And Derivatives Thereof>>

For the purpose of preventing any deterioration in sensitivity and improving the resist pattern shape, the post-exposure temporal stability, and the like, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in combination of two or more kinds thereof.

In a case where the resist composition contains the component (F), the content of the component (E) is preferably in a range of 0.01 to 5 parts by mass and more preferably in a range of 0.05 to 3 parts by mass with respect to 100 parts by mass of the component (A1). In a case where the content thereof is in the above-described range, the sensitivity, lithography characteristics, and the like are improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived front acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl 1-adamantyl (meth)acrylate is preferable, and a constitutional unit derived from 1-ethyl-1-cyclooctyl (meth)acrylate is more preferable.

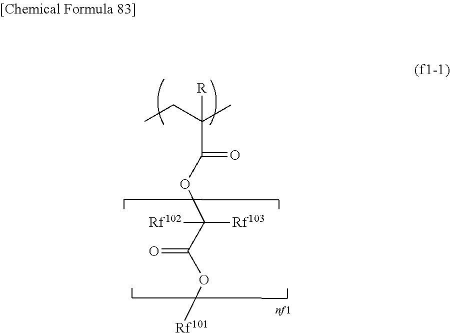

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other, nf¹ represents an integer of 0 to 5, and Rf¹⁰¹ represents an organic group having a fluorine atom].

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these. Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and particularly preferably 60% or greater thereof are fluorinated from the viewpoint of increasing the hydrophobicity of the resist film during immersion exposure.

Among examples. Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is satisfactory.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A1).

### <<Organic Solvent Component (S)>>

The resist composition according to the present embodiment may be produced by dissolving the resist materials in an organic solvent component (hereinafter, referred to as a “component (S)”).

The component (S) may be any organic solvent which can dissolve each component to be used to obtain a uniform solution, and an optional organic solvent can be appropriately selected from those which have been known as solvents of a chemically amplified resist composition and then used in the related art.

In the resist composition of the present embodiment, the component (S) may be used alone or in the form of a mixed solvent of two or more kinds thereof. Among these, PGMEA, POME, γ-butyrolactone, EL, or cyclohexanone is preferable.

Further, a mixed solvent obtained by mixing PGMEA with a polar solvent is also preferable as the component (S). The blending ratio (mass ratio) may be appropriately determined in consideration of the compatibility or the like between PGMEA and the polar solvent.

Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist, composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component (S), impurities and the like may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

In the resist composition of the present embodiment described above, a resin component (component (A1)) having a constitutional unit (a0) derived from a compound represented by General Formula (a0-m), which is a constitutional unit that generates an acid upon exposure, is employed. The constitutional unit (a0) includes, in its side chain, a linking group with an amide structure (—C(═O)—N(R⁰)—) and an aromatic ring (R^{ar}) with an iodine atom bonded as a substituent.

In the resist composition of the present embodiment, an iodine atom, which is a high-absorption element, is bonded to an aromatic ring (R^{ar}) and exists within the same constitutional unit as the sulfonic acid structure. As a result, secondary electrons generated during exposure can be utilized more efficiently.

In component (A1), a constitutional unit that generates acid upon exposure is introduced, and because a linking group with an amide structure is present within this constitutional unit, the interaction between resins suppresses the concentration of acid generated by exposure within the resist (or polymer compound) composition.

In addition, the effect is achieved by the combination of the efficient use of electrons by iodine atoms, the suppression of acid diffusion by the “linking group with an amide structure having moderate basicity,” and the “polymer-bound acid generator structure.”

By employing the specific component (A1) as described, the aforementioned effects are synergistically enhanced. According to the resist composition of the present embodiment, it is considered that high sensitivity in forming the resist pattern and improvements in lithography characteristics such as dimensional uniformity are achieved. Additionally, the process margin, including tight exposure margin, is broadened.

### (Resist Pattern Formation Method)

A resist pattern formation method according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist, pattern formation method, a resist pattern formation method by performing processes as described below is an exemplary example.

First, a support is coated with the resist composition of the present embodiment using a spinner or the like, and a bake (post apply bake (PAB)) treatment is performed, for example, under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing or the like with an electron beam without using a mask pattern, a bake treatment (post-exposure bake PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. A bake treatment (post bake) may be conducted after the developing treatment in some cases.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, for example, copper, aluminum, nickel, or gold can be used.

The wavelength to be used for light exposure is not particularly limited and the exposure can be conducted using radiation such as an ArF excimer laser, a KrF excimer laser, an F₂ excimer laser, extreme ultraviolet (EUV) rays, vacuum ultraviolet rays (VUV), electron beams (EB), X-rays, and soft X-rays.

The resist pattern formation method according to the present embodiment is a useful method in which the resist film is exposed to extreme ultraviolet (EUV) rays or electron beams (EB) in the step of exposing the resist film to light.

The method of exposing the resist film to light may be general exposure (dry exposure) conducted in air or an inert gas such as nitrogen, or liquid immersion lithography.

The liquid immersion lithography is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, for example, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the resist composition mentioned above is used, it is possible to achieve high sensitivity and improve lithography characteristics such as dimensional uniformity when forming the resist pattern. Additionally, a wide process margin allows for the stable formation of a resist pattern with a favorable shape.

Various materials that are used in the resist composition according to the above-described embodiment and the resist pattern formation method according to the above-described embodiment (for example, a resist solvent, a developing solution, a rinse solution, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K. Ca, Fe, Cu. Mn, Mg. A1. Cr, Ni, Zn. Ag, Sn, Pb, Li. and salts thereof. The content of the impurities contained in these materials is preferably 200 ppb or less, more preferably 1 ppb or less, still more preferably 10 parts per trillion (ppt) or less, and particularly preferably 10 ppt or less, where it is most preferable that the impurities are substantially free (less than or equal to the detection limit of the measuring device).

### (Compound)

A compound according to a third aspect of the present invention is a compound represented by General Formula (a0-m) (hereinafter, also referred to as “compound (a0-m)”).

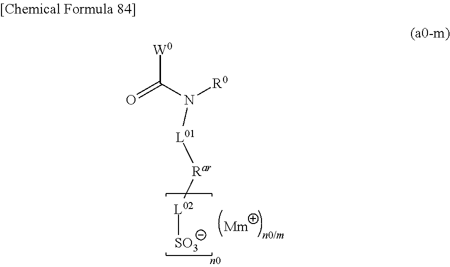

[In the formula, W⁰ represents a polymerizable group-containing group. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent, linking group, n0 represents an integer of 1 or more as long as an atomic valence allows, m represents an integer of 1 or more, and (M^{m+})_{n0/m} represents a counter cation.

The description of the compound represented by General Formula (a0-m) (compound (a0-m)) is the same as the description of the compound represented by General Formula (a0-m) described in the section of <<Constitutional unit (a0)>> above.

Hereinafter, preferred specific examples of the anionic moiety of the compound (0-m) will be shown. In the formulae shown below, R^{α} represents a hydrogen atom, a methyl group, or a trifluoromethyl group.

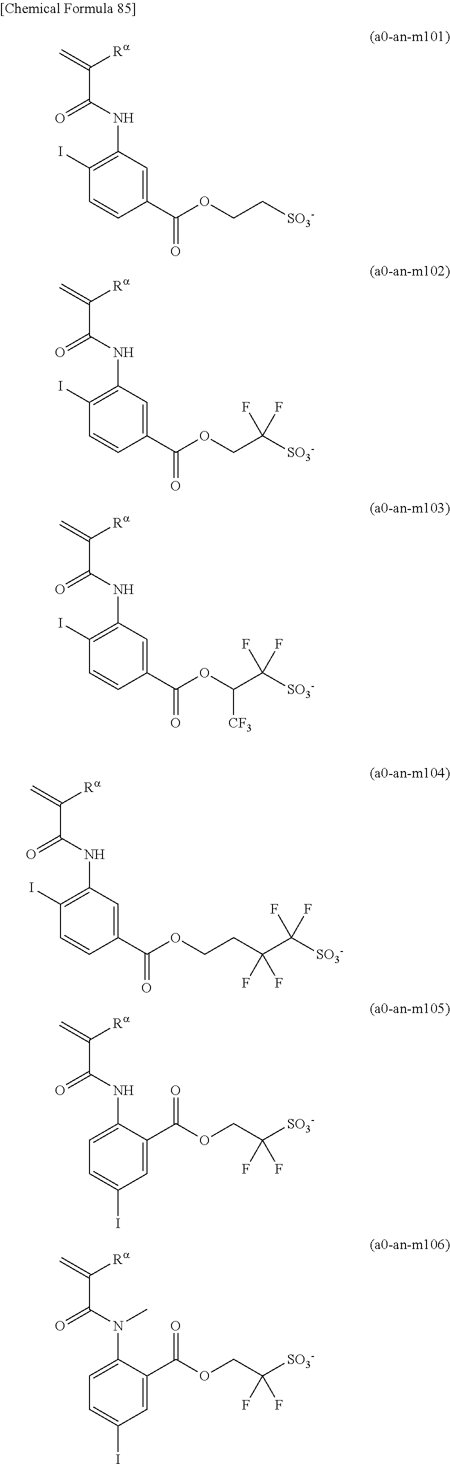

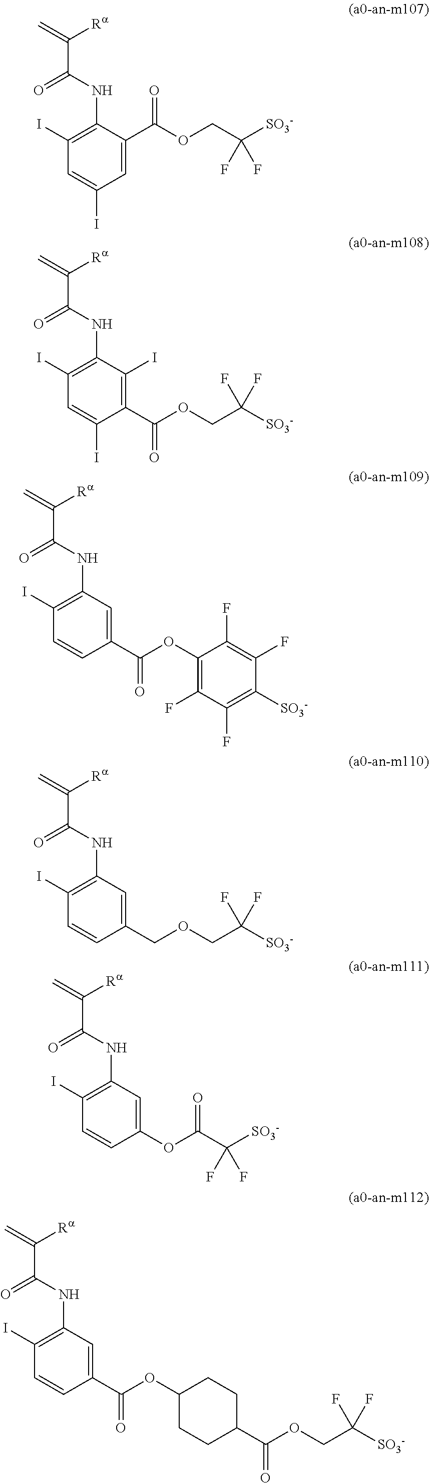

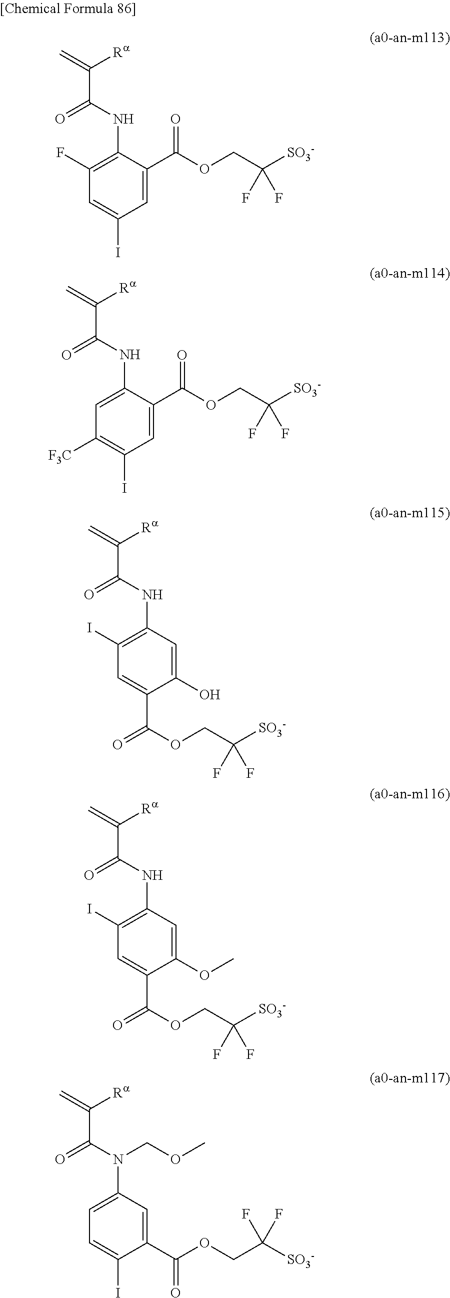

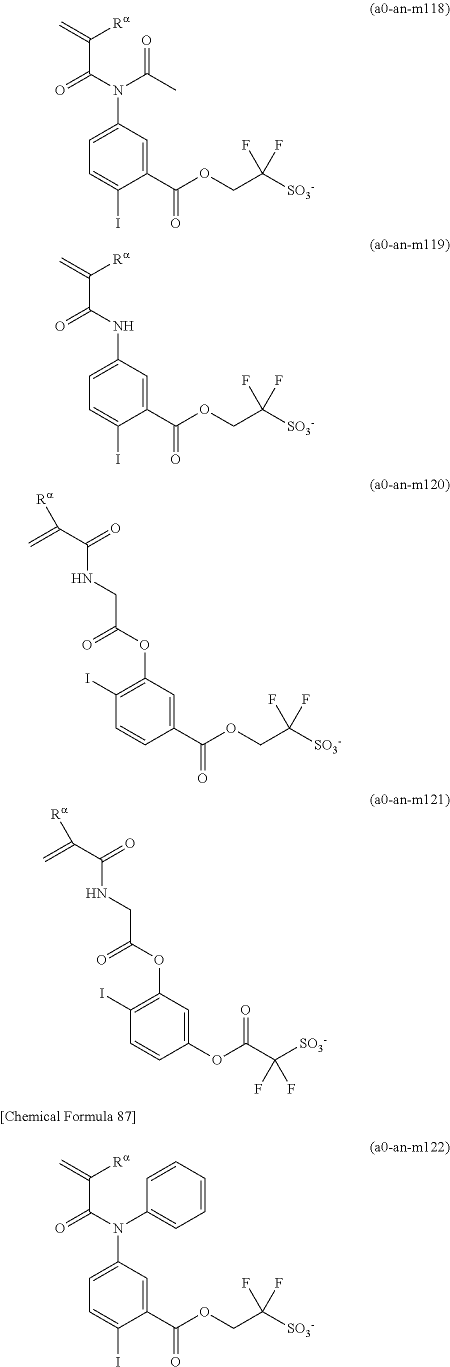

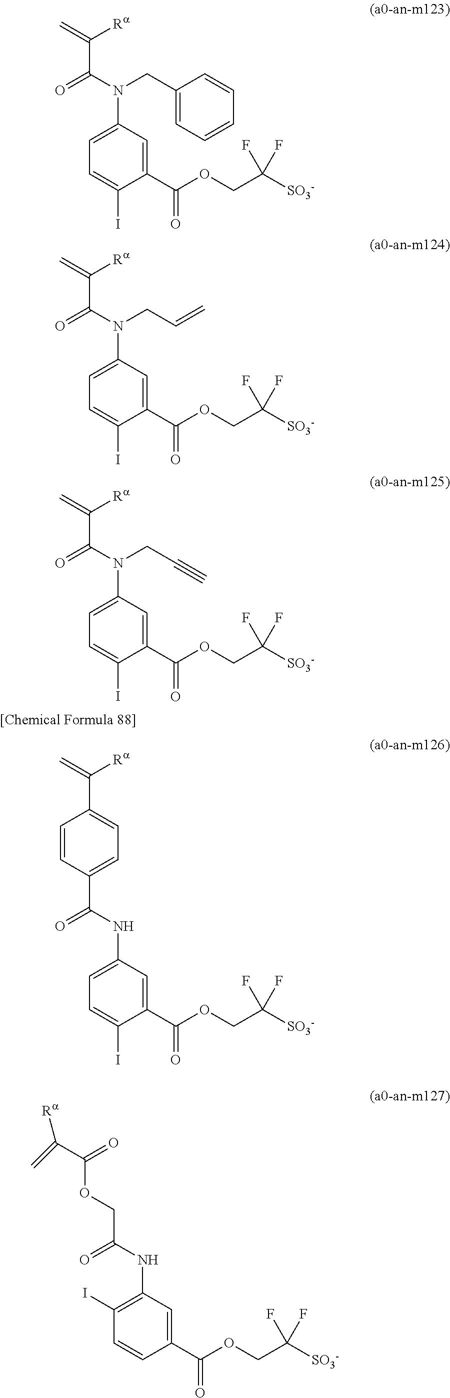

Suitable examples of the compound (a0-m) include a compound represented by General Formula (a0-m1).

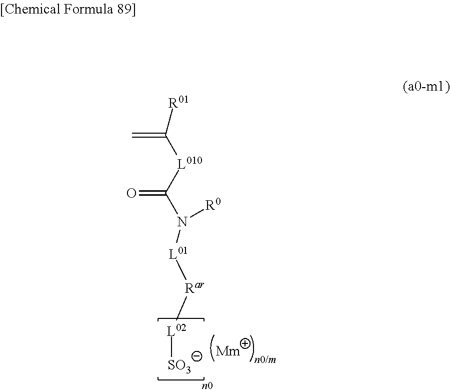

[In the formula, R⁰¹ represents an alkyl group having 1 to 5 carbon atoms, a halogenated alkyl group having 1 to 5 carbon atoms, a halogen atom, or a hydrogen atom, L⁰¹⁰ represents a divalent linking group or a single bond. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, in represents an integer of 1 or more, and (M^{m+})_{n0/m} represents a counter cation.

In the formula (a0-m1), R⁰¹, L⁰¹⁰, R⁰, L⁰¹, R^{ar}, L⁰², n0, m, and (M^{m+})_{n0/m} are the same as R⁰¹, L⁰¹⁰, R⁰, L⁰¹, R^{ar}, L⁰², n0, m, and (M^{m+})_{n0/m} in Formula (a0-u1) described above.

As preferred specific examples of the anion moiety of the compound represented by the formula (a0-m1), the anions respectively represented by the chemical formulas (a0-an-m101) to (a0-an-m125) (where L⁰¹⁰ is a single bond); and the anions respectively represented by the Chemical Formulae (a0-an-m126) and (a0-an-n127) (where L⁰¹⁰ is a divalent linking group) can be mentioned.

Alternatively, suitable examples of the compound (a0-m) include a compound represented by General Formula (a0-m2).

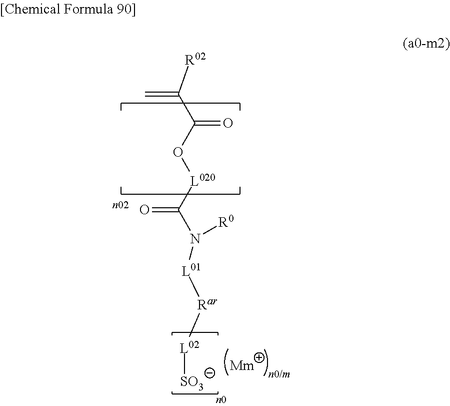

[In the formula, R⁰² represents an alkyl group having 1 to 5 carbon atoms, a halogenated alkyl group having 1 to 5 carbon atoms, a halogen atom, or a hydrogen atom. L⁰²⁰ represents a divalent linking group, n02 represents an integer of 0 to 2. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, m represents an integer of 1 or more, and (M^{m+})_{n0/m} represents a counter cation.

In the formula (a0-m2), R⁰², L⁰²⁰, n02, R⁰, L⁰¹, R^{ar}, L⁰², n0, m, and (M^{m+})_{n0/m} are the same as R⁰², L⁰²⁰, n02, R⁰, L⁰¹, R^{ar}, L⁰², n0, m, and (M^{m+})_{n0/m} in the formula (a0-u2).

As preferred specific examples of the anion moiety of the compound represented by the formula (a0-m2), the anions represented by the chemical formulas (a0-an-m101) to (a0-an-m125), and (a0-an-m127) are mentioned.

Alternatively, suitable examples of the compound (a0-m) include a compound represented by General Formula (a0-m3),

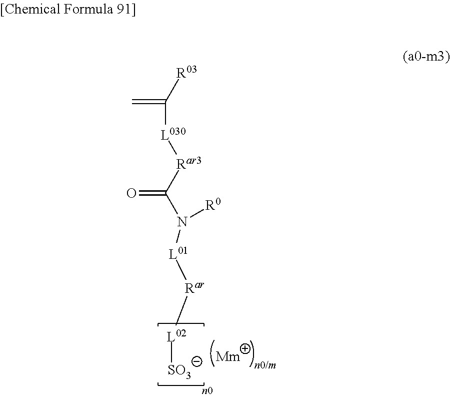

[In the formula, R⁰³ represents an alkyl group having 1 to 5 carbon atoms, a halogenated alkyl group having 1 to 5 carbon atoms, a halogen atom, or a hydrogen atom. L⁰³⁰ represents a divalent linking group or a single bond. R^{ar3} represents an aromatic ring, which may have a substituent. R⁰ represents a hydrocarbon group, an acyl group, an alkoxyalkyl group, or a hydrogen atom. L⁰¹ represents a divalent linking group or a single bond. R^{ar} represents an aromatic ring to which an iodine atom is bonded as a substituent. A substituent other than the iodine atom may be bonded to the aromatic ring. L⁰² represents a divalent linking group, n0 represents an integer of 1 or more as long as an atomic valence allows, in represents an integer of 1 or more, and (M^{m+})_{n0/m} represents a counter cation.

In the formula (a0-m3), R⁰³, L⁰³⁰, R^{ar3}, R⁰, L⁰¹, R^{ar}, L⁰², n0, m, and (M^{m+})_{n0/m} are the same as R⁰³, L⁰³⁰, R^{ar3}, R⁰, L⁰¹, R^{ar}, L⁰², n0, m, and (M^{m+})_{n0/m} in the above formula (a0-u3).

A preferred specific example of the anion moiety of the compound represented by Formula (a0-m3) includes the anion represented by Chemical Formula (a0-an-m126).

The compounds preferably mentioned above (a0-m), the compounds represented by General Formula (a0-m1), the compounds represented by General Formula (a0-m2), and the compounds represented by General Formula (a0-m3) are further preferred when R^{ar} in General Formula is a benzene ring with two or more iodine atoms as substituents. In this case, preferred specific examples of the anion moiety of the compound include the anions represented by Chemical Formulae (a0-an-m107) and (a0-an-m108).

Examples of the counter cation in (M^{m+})_{n0/m} for the cationic moiety of the compound (a0-m) include an onium cation and a metal cation.

As the onium cation in (M^{m+})_{n0/m}, examples include sulfonium cation, iodonium cation, and ammonium cation. The sulfonium cations and iodonium cations mentioned here are similar to those exemplified in the description of the [cation moiety] mentioned above.

Examples of the ammonium cation in (M^{m+})_{n0/m} include an organic ammonium cation represented by General Formula (ca-4).

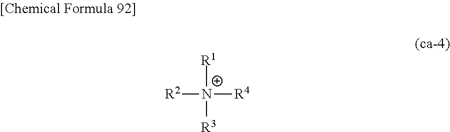

[In the formula, R¹ to R⁴ each independently represent a hydrocarbon group which may have a substituent. Alternatively, at least two of R¹ to R⁴ may be bonded to each other to form an alicyclic structure together with the nitrogen atom in the formula].

In General Formula (ca-4), the hydrocarbon groups as R¹ to R⁴ are each independently preferably a hydrocarbon group having 1 to 15 carbon atoms and more preferably a hydrocarbon group having 1 to 10 carbon atoms. Further, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group as R¹ to R⁴ include a linear or branched alkyl group and a cyclic hydrocarbon group.

The linear or branched alkyl group is preferably a linear or branched alkyl group having 1 to 10 carbon atoms and more preferably a linear or branched alkyl group having 1 to 5 carbon atoms.

The cyclic hydrocarbon group may be an alicyclic hydrocarbon group or an aromatic hydrocarbon group.

The alicyclic hydrocarbon group is preferably a group obtained by removing one hydrogen atom from a monocycloalkane. The monocycloalkane has preferably 3 to 6 carbon atoms, and specific examples thereof include cyclopentane and cyclohexane. The aromatic hydrocarbon group is preferably a phenyl group or a benzyl group.

Examples of the substituent which may be included in the hydrocarbon group as R¹ to R⁴ include an alkoxy group, a hydroxyl group, an oxo group (═O), and an amino group.

Specific examples of the suitable cation represented by General Formula (ca-4) include cations each represented by Chemical Formulae (ca-4-1) to (ca-4-4) described below.

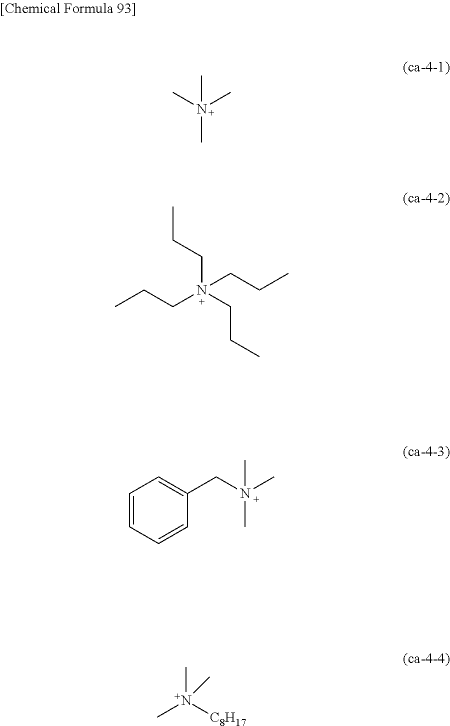

Examples of the metal cation in (M^{m+})_{n0/m} include alkali metal ions such as a sodium ion and a lithium ion; alkaline earth metal ions, and a rubidium ion, a strontium ion, and a yttrium ion.

From the viewpoint of the resist material application, in the compound (a0-m) suitably mentioned above, the compound represented by General Formula (a0-m1), the compound represented by General Formula (a0-m2), and the compound represented by General Formula (a0-m3), the cationic moiety (M^{m+})_{n0/m} is preferably a sulfonium cation or an iodonium cation, more preferably an onium cation each represented by General Formulae (ca-1) to (ca-3), and particularly preferably a sulfonium cation represented by General Formula (ca-1).

Specific examples of the compound (a0-m) are shown below, but the present invention is not limited thereto.

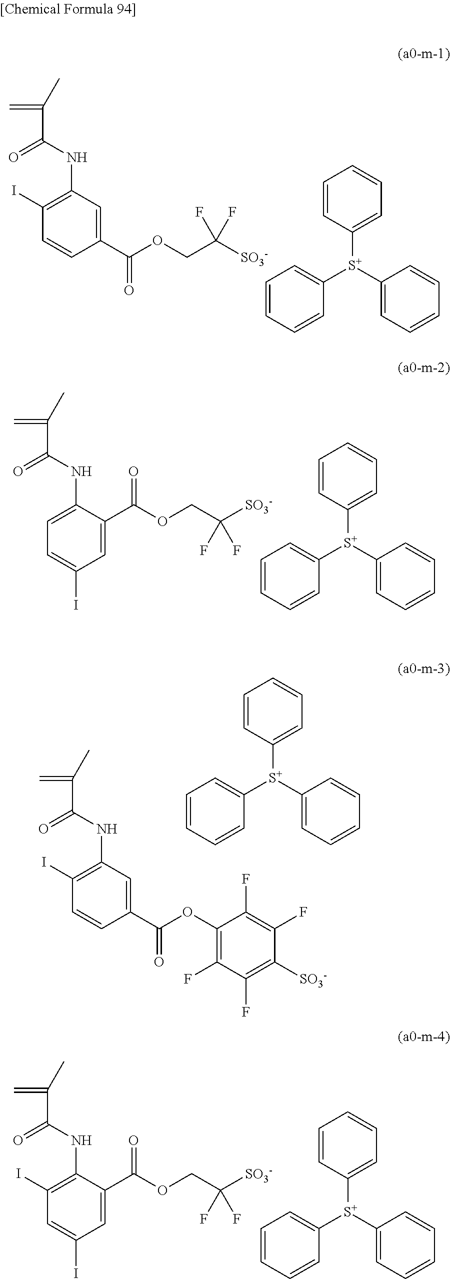

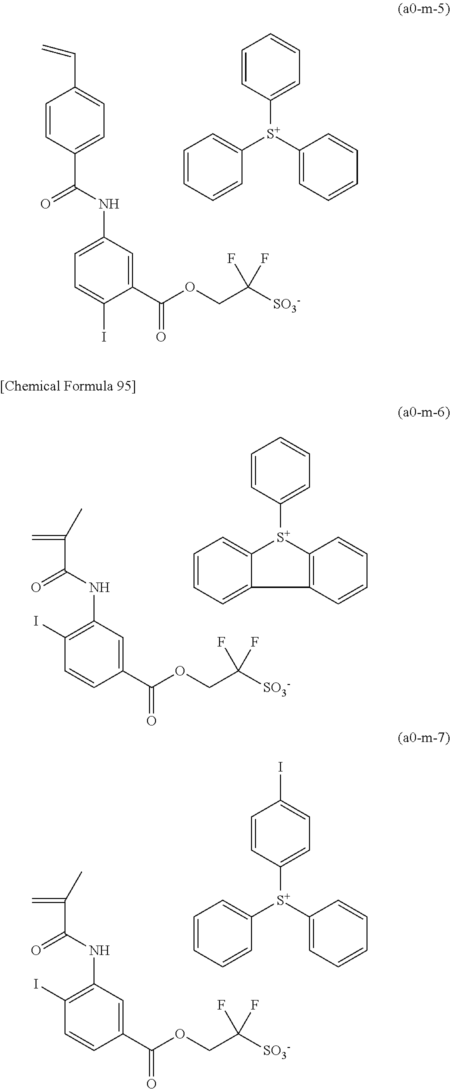

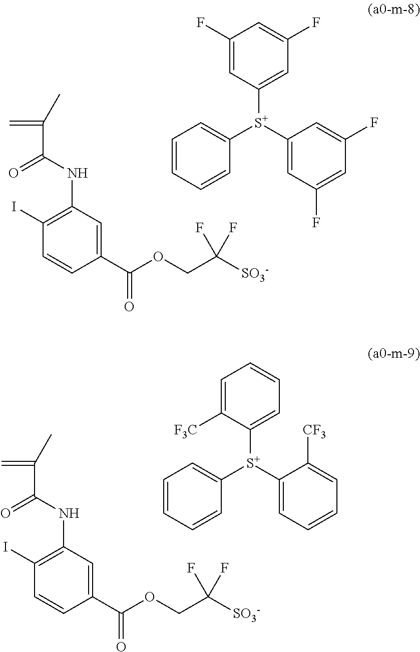

[Method of Producing Compound (a0-m)]

The compound (a0-m) of the present embodiment can be produced by appropriately combining known methods. For example, as [Synthesis Example 1: synthesis of compound (a0-m-1)] described later, the compound (a0-m) can be produced by selecting a raw material (raw material corresponding to W⁰, raw material corresponding to R^{ar}, sulfonic acid-based compound, or the like) corresponding to the desired compound, and by using a method of producing that includes a first step (such as condensation), a second step (such as condensation), and a third step (such as salt exchange).

The compound according to the present embodiment described above can be used as a raw material monomer of a polymer compound according to a fourth aspect described later.

### (Polymer Compound)

The fourth aspect of the present invention relates to a polymer compound having a constitutional unit derived from the compound according to the third aspect of the present invention described above.

The constitutional unit derived from the compound according to the third aspect is a constitutional unit derived from the compound represented by General Formula (a0-m), and is the same as the constitutional unit (a0) described above.

The description of the polymer compound according to the fourth aspect is the same as the description of the component (A1).

A polymer compound according to the fourth aspect, namely, a polymer compound having a constitutional unit (constitutional unit (a0)) derived from a compound represented by the general formula (a0-m), can be used as a base material component of the resist composition according to the first aspect. In addition, the polymer compound having the constitutional unit (a0), the constitutional unit (a10) described above, and the constitutional unit (a1) described above is particularly useful as a resist material for lithography, which is exposed to EUV or EB. Alternatively, the polymer compound having the constitutional unit (a0), the constitutional unit (a10) described above, the constitutional unit (a1) described above, and the constitutional unit (a2) described above is particularly useful as a resist material for lithography, which is exposed to EUV or EB.

The polymer compound according to this fourth aspect can serve as a useful resin component in the base material component of a resist composition.

### EXAMPLES

Hereinafter, the present invention will be described in more detail based on examples, but the present invention is not limited to these examples.

### Synthesis Example of Compound

### Synthesis Example 1: Synthesis of Compound (a0-m-1)

### First Step:

11.4 g of methacryloyl chloride and 26.3 g of 3-amino-4-iodobenzoic acid were added to 100 g of tetrahydrofuran (THF), and 12.1 g of triethylamine (TEA) was added dropwise under ice-cooling. After stirring for 1 hour under ice cooling, 100 g of 5% hydrochloric acid was added. By purifying the precipitate using column chromatography, 19.9 g (60%) of compound (m-11) was obtained.

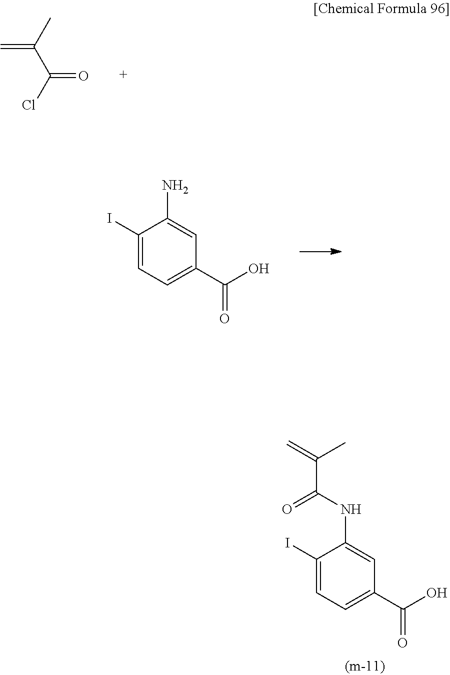

### Second Step:

11.1 g of the compound (m-11), 10.3 g of the compound (m-12), 7.5 g of diisopropylcarbodiiimide (DIC), and a catalytic amount of dimethylaminopyridine (DMAP) were stirred in 200 g of dichloromethane at room temperature for 6 hours. After removing the insoluble matter by filtration, the solvent was distilled off, and the obtained residue was purified by silica gel column chromatography to obtain 14.5 g (70%) of a compound (a0-m-1pre).

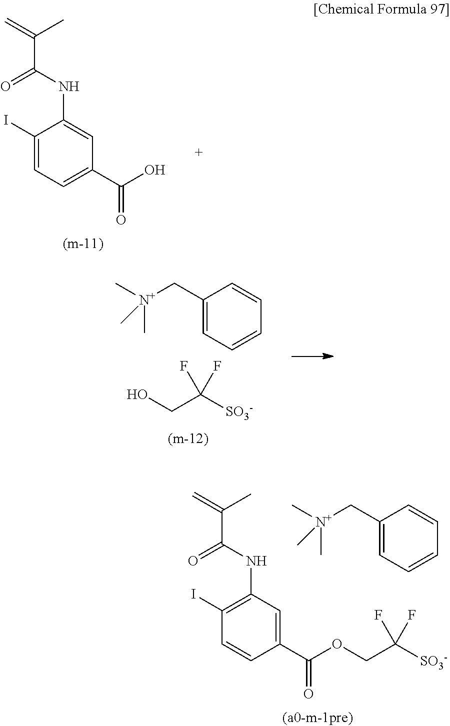

### Third Step:

12.5 g of compound (a0-m-1pre) and 7.5 g of triphenylsulfonium bromide (compound A) were dissolved in 100 g of dichloromethane, 100 g of water was added thereto, and the mixture was stirred at room temperature for 1 hour, and then the aqueous layer was removed. After repeatedly washing the organic layer with water, the solvent was removed by distillation and dried to obtain 14.0 g (95%) of compound (a0-m-1).

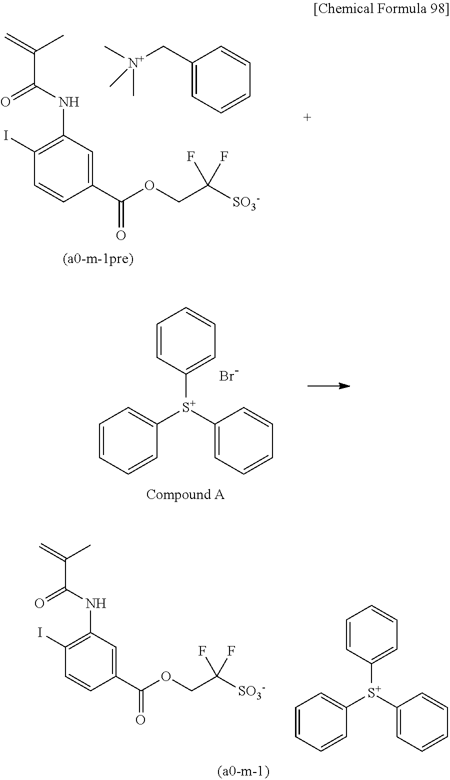

The obtained compound (a0-m-1) was subjected to NMR measurement, and the structure thereof was identified from the following results.

¹H-NMR (dmso-d6, 400 MHz): δ(ppm)=11.1 (s, —NH—, 1H), 7.9-8.0 (m, —Ar, 2H), 7.7-7.9 (m, -Ph, 15H), 7.4 (m, —Ar, 1H), 6.1 (s, ═CH₂, 1H), 5.7 (s, ═CH₂, 1H), 4.6-4.7 (m, —CH₂—, 2H), 1.9 (s, —CH3, 3H)

### Synthesis Example 2: Synthesis of Compound (a0-m-2)

In the first step of Synthesis Example 1 described above, compound (a0-m-2) was obtained in the same manner as in the first to third steps of Synthesis Example 1, except that 3-amino-4-iodobenzoic acid was replaced with 2-methylamino-5-iodobenzoic acid.

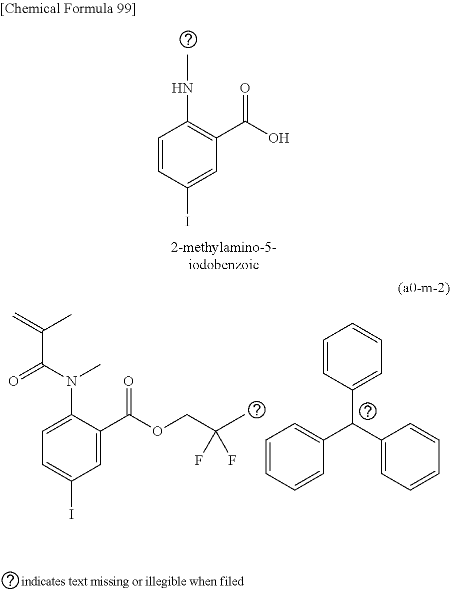

The obtained compound (at-m-2) was subjected to NMR measurement, and the structure thereof was identified from the following results.

¹H-NMR (dmso-d6, 400 MHz): δ(ppm)=7.7-7.9 (m, -Ph, 15H), 7.6 (s, —Ar, 1H), 7.3 (m, —Ar, 11H), 6.4 (m, —Ar, 1H), 6.1 (s, ═CH2, 1H), 5.7 (s, ═CH2, 1H), 4.6-4.7 (m, —CH2-, 2H), 3.3 (s, —CH3, 3H), 1.9 (s, CH3, 3H)

### Synthesis Example 3: Synthesis of Compound (a0-m-3)

In the second step of Synthesis Example 1 described above, compound (m-12) was replaced with compound (m-32), and compound (a0-in-3) was obtained in the same manner as in the first to third steps of Synthesis Example 1.

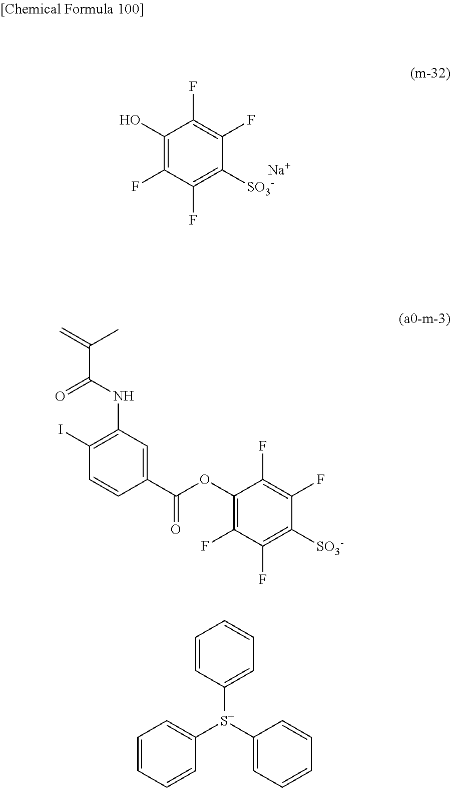

The obtained compound (a0-m-3) was subjected to NMR measurement, and the structure thereof was identified from the following results.

¹H-NMR (dmso-d6, 400 MHz): δ(ppm)=11.1 (s, —NH—, 1H), 7.9-8.0 (m, —Ar, 2H), 7.7-7.9 (m, -Ph, 15H), 7.4 (m, —Ar, 1H), 6.1 (s, ═CH2, 1H), 5.7 (s, ═CH2, 1H), 1.9 (s, —CH3, 3H)

### Synthesis Example 4: Synthesis of Compound (a0-m-4)

In the first step of Synthesis Example 1 described above, compound (a0-m-4) was obtained in the same manner as in the first to third steps of Synthesis Example 1, except that 3-amino-4-iodobenzoic acid was replaced with 2-amino-3,5-diiodobenzoic acid.

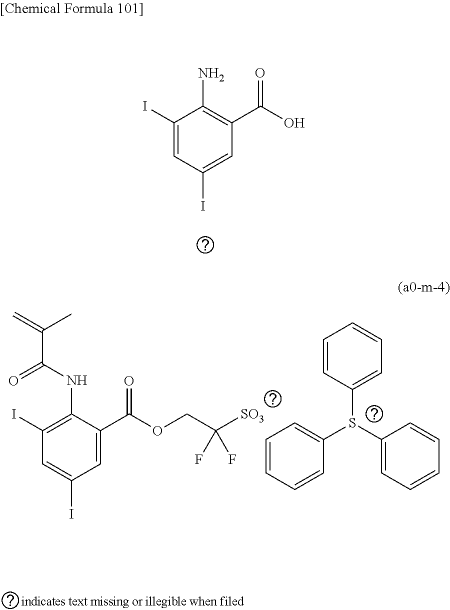

The obtained compound (a0-m-4) was subjected to NMR measurement, and the structure thereof was identified from the following results.

¹H-NMR (dmso-d6, 400 MHz): δ(ppm)=11.1 (s, —NH—, 1H), 8.6 (s, —Ar, 1H), 8.3 (s, —Ar, 1H), 7.7-7.9 (m, -Ph, 15H), 6.1 (s, ═CH2, 1H), 5.7 (s, ═CH2, 1H), 4.6-4.7 (m, —CH2-, 2H), 1.9 (s, —CH3, 3H)

### Synthesis Example 5: Synthesis of Compound (a0-m-5)

In the first step of Synthesis Example 1 described above, the compound (a0-m-5) was obtained in the same manner as in the first to third steps of Synthesis Example 1, except that methacryloyl chloride was replaced with 4-vinylbenzoic acid chloride, and 3-amino-4-iodobenzoic acid was replaced with 3-amino-6-iodobenzoic acid.

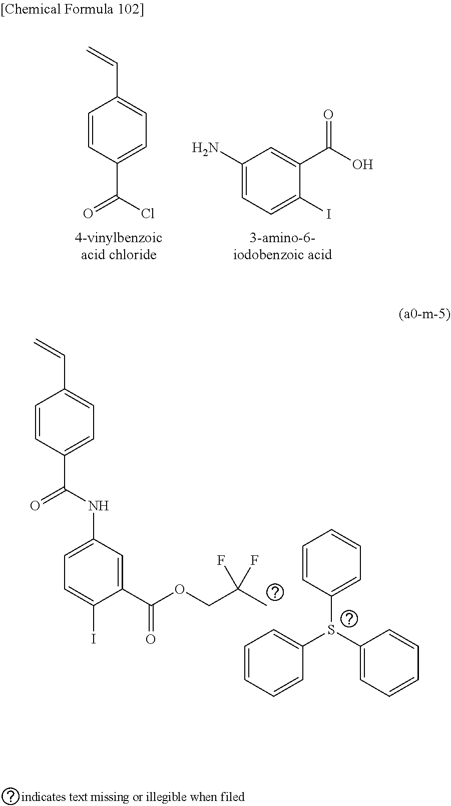

The obtained compound (a0-m-5) was subjected to NMR measurement, and the structure thereof was identified from the following results.

¹H-NMR (dmso-d6, 400 MHz): δ(ppm) 11.1 (s, —NH—, 1H), 7.9 (m, —Ar, 2H), 7.7-7.9 (m, -Ph, 16H), 7.6 (m, —Ar, 2H), 7.5-7.6 (m, —Ar, 2H), 6.1 (s, ═CH2, 1H), 5.7 (s, ═CH2, 1H), 4.6-4.7 (m, —CH2-, 2H), 1.9 (s, —CH3, 3H)

### Synthesis Example 6: Synthesis of Compound (a0-m-6)

A compound (a0-m-6) was obtained in the same manner as in the first to third steps of Synthesis Example 1, except that in the third step of Synthesis Example 1, triphenylsulfonium bromide (compound A) was changed to the compound B.

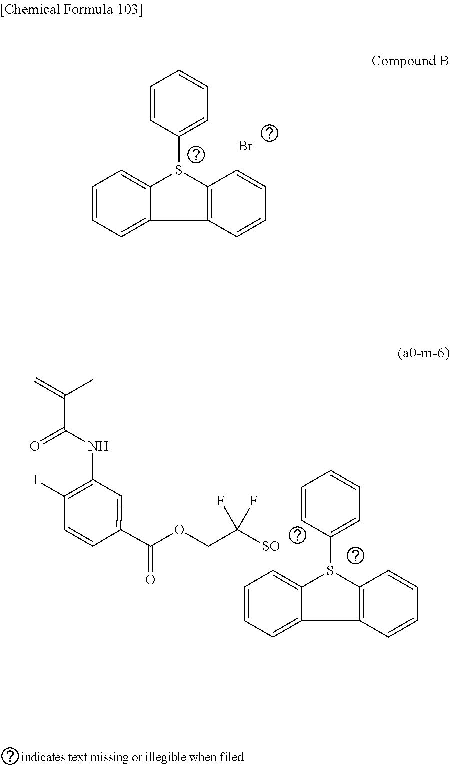

The obtained compound (a0-m-6) was subjected to NMR measurement, and the structure thereof was identified from the following results.

¹H-NMR (dmso-d6, 400 MHz): δ(ppm)=11.1 (s, —NH—, 1H), 8.5 (m, —Ar, 2H), 8.4 (m, —Ar, 2H), 7.9-8.0 (m, —Ar, 4H), 7.6-7.8 (m, —Ar, 7H), 7.4 (m, —Ar, 1H), 6.1 (s, ═CH2, 1H) 5.7 (s, ═CH2, 1H), 4.6-4.7 (m, —CH₂—, 2H), 1.9 (s, —CH3, 3H)

### Synthesis Example 7: Synthesis of Compound (a0-m1-7)

A compound (a0-m-7) was obtained in the sane manner as in the first to third steps of Synthesis Example 1, except that in the third step of Synthesis Example 1, triphenylsulfonium bromide (compound A) was changed to the compound C.

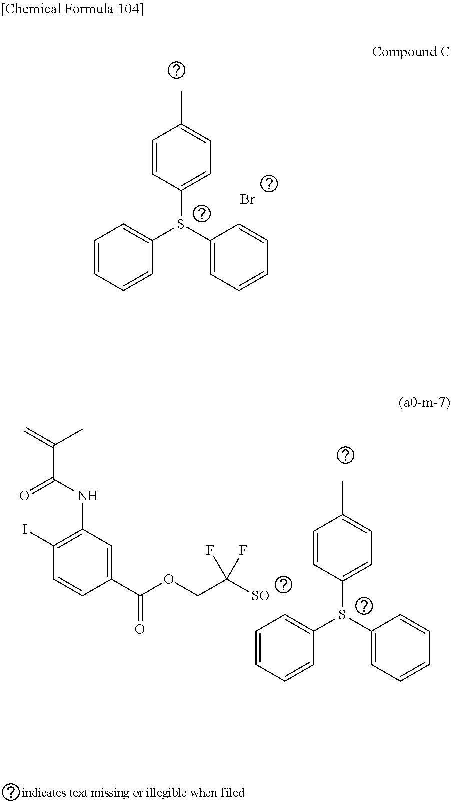

The obtained compound (a0-m-7) was subjected to NMR measurement, and the structure thereof was identified front the following results.

¹H-NMR (dmso-d6, 400 MHz): δ(ppm)=11.1 (s, —NH—, 1H), 8.2 (m, —Ar, 2H), 7.9-8.0 (m, —Ar, 2H), 7.7-7.9 (m, —Ar, 10H), 7.5 (m, —Ar, 2H), 7.4 (m, —Ar, 1H), 6.1 (s, ═CH2, 1H), 5.7 (s, ═CH2, 1H), 4.6-4.7 (m, —CH₂—, 2H), 1.9 (s, —CH3, 3H)

### Synthesis Example 8: Synthesis of Compound (a0-m-8)

A compound (a0-m-8) was obtained in the same manner as in the first to third steps of Synthesis Example 1, except that in the third step of Synthesis Example 1, triphenylsulfonium bromide (compound A) was changed to the compound D.

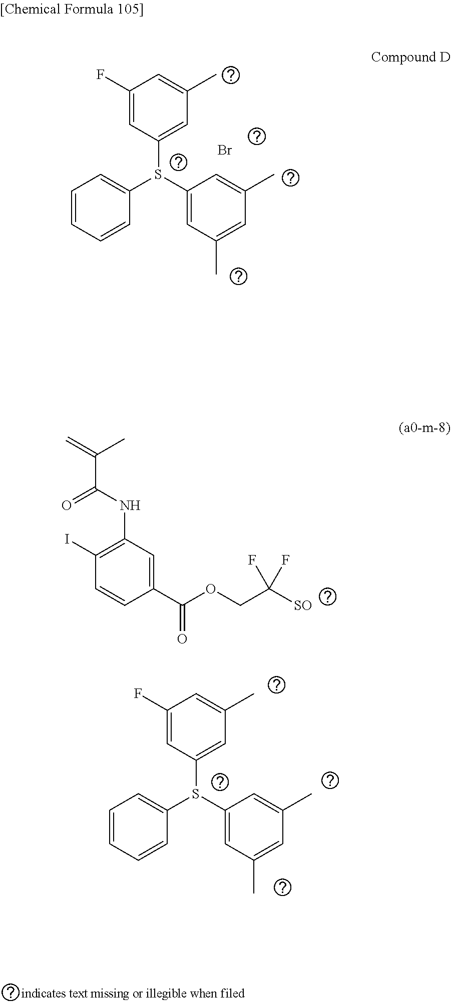

The obtained compound (a0-m-8) was subjected to NMR measurement, and the structure thereof was identified from the following results.

¹H-NMR (dmso-d6, 400 MHz): δ(ppm)=11.1 (s, —NH—, 1H), 7.7-8.0 (m, —Ar, 13H), 7.4 (m, —Ar, 1H), 6.1 (s, ═CH2, 1H), 5.7 (s, ═CH2, 1H), 4.6-4.7 (m, —CH₂—, 2H), 1.9 (s, —CH3, 3H)

### Synthesis Example 9: Synthesis of Compound (a0-m-9)

A compound (a0-m-9) was obtained in the same manner as in the first to third steps of Synthesis Example 1, except that in the third step of Synthesis Example 1, triphenylsulfonium bromide (compound A) was changed to the compound E.

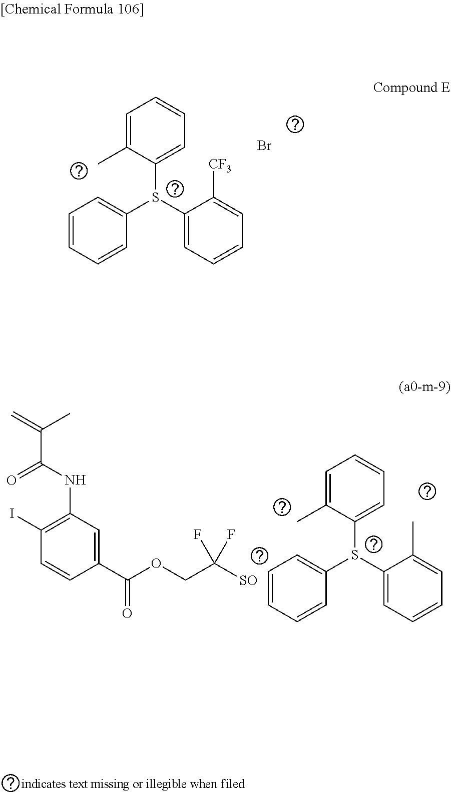

The obtained compound (a0-m-9) was subjected to NMR measurement, and the structure thereof was identified from the following results.

¹H-NMR (dmso-d6, 400 MHz): δ(ppm)=11.1 (s, —NH—, 1H), 8.3 (m, —Ar, 2H), 8.1-8.2 (m, —Ar, 2H), 7.9-8.0 (m, —Ar, 7H), 7.6 (m, —Ar, 2H), 7.4 (m, —Ar, 1H), 6.1 (s, ═CH₂, 1H), 5.7 (s, ═CH2, 1H), 4.6-4.7 (m, —CH2-, 2H), 1.9 (s, —CH3, 3H)

### Synthesis Example of Polymer Compound

### [Synthesis of Polymer Compound (A1-1)]

4.4 g of the compound (a0-m-1), 3.0 g of the compound (a10-01pre), 5.3 g of the compound (a1-01), and 1.1 g of dimethyl azobis(isobutyrate) (V-601) as a polymerization initiator were dissolved in 40 g of methyl ethyl ketone (MEK), and the solution was stirred at 80° C. for 6 hours in a nitrogen atmosphere.

Thereafter, 2.0 g of acetic acid and 60 g of methanol were added to the reaction solution, and the mixture was stirred at 30° C. for 18 hours. After completion of the reaction, the obtained reaction solution was precipitated in 600 g of heptane, followed by washing. The obtained white solid was filtered and then subjected to reduce pressure drying overnight to obtain 6.2 g of a polymer compound (A1-1) as a target substance.

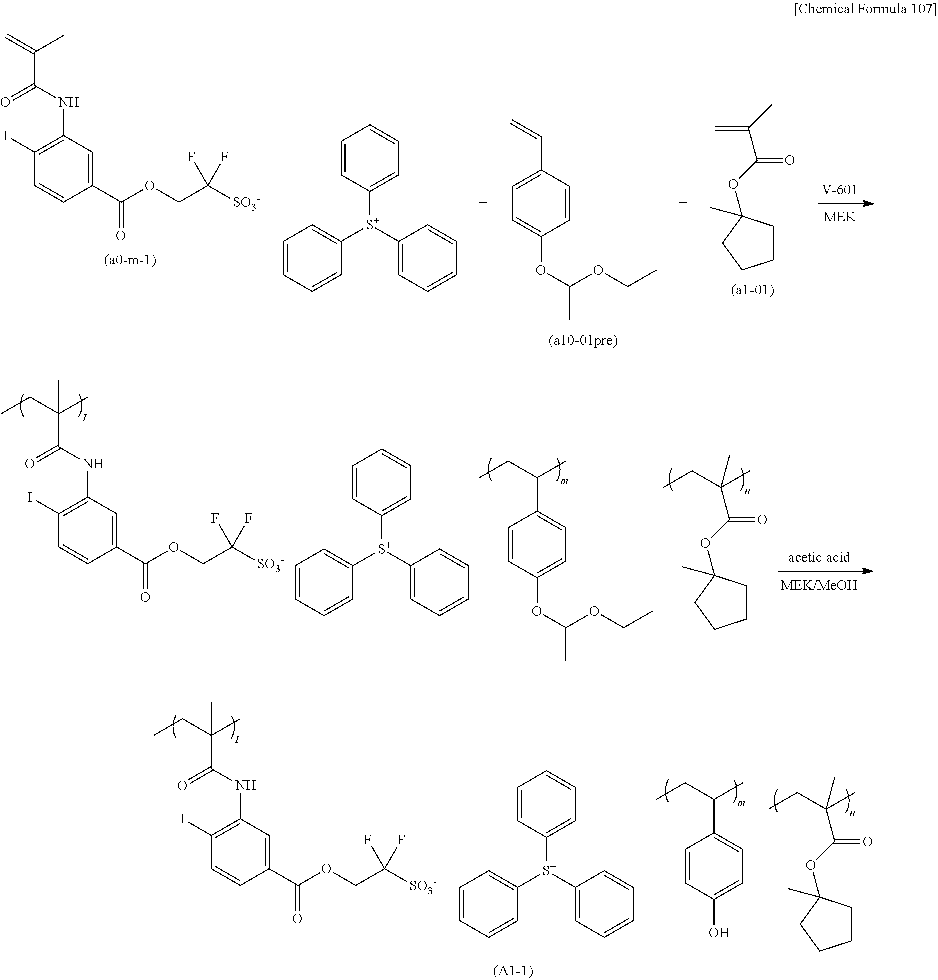

### [Synthesis of Polymer Compounds (A1-2) to (A1-14), Polymer Compounds (A2-1) to (A2-4)]

Each of polymer compounds (A1-2) to (A1-14) and polymer compounds (A2-1) to (A2-4) was obtained by the same synthesis method (radical polymerization and deprotection reaction) as in [Synthesis of polymer compound (A1-1)] described above using the corresponding raw material monomers.

The structures of the corresponding raw material monomers and the obtained respective polymer compounds are shown below. In each formula, l, m, n, and p represent the compositional ratios of each constitutional unit.

The compound (a10-01pre) is a precursor of the constitutional unit (a10-01), the compound (a10-02pre) is a precursor of the constitutional unit (a10-02), and the compound (a10-03pre) is a precursor of the constitutional unit (a10-03).

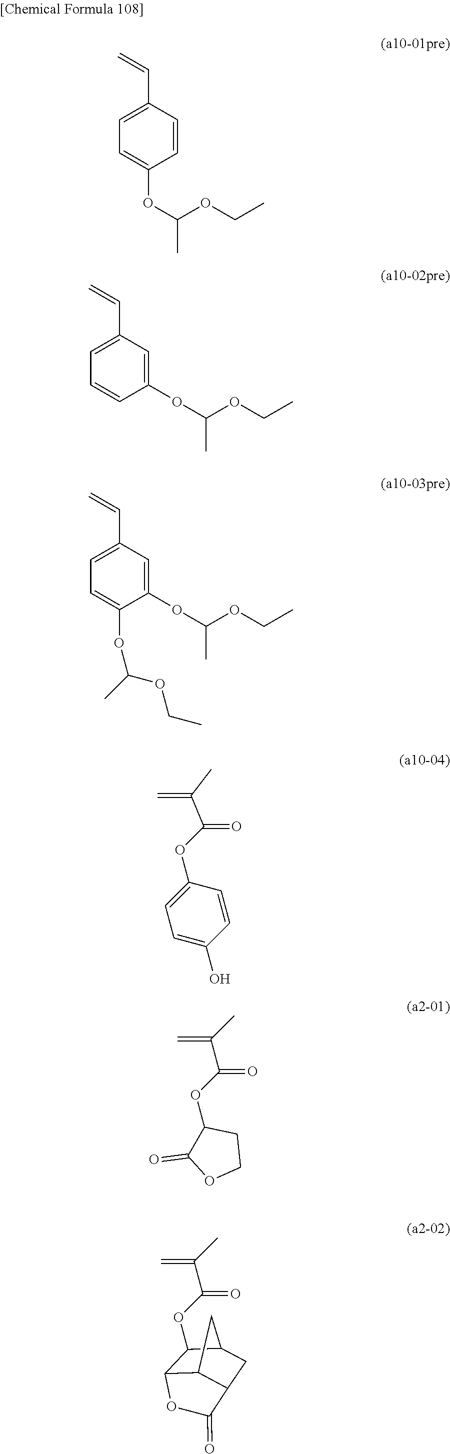

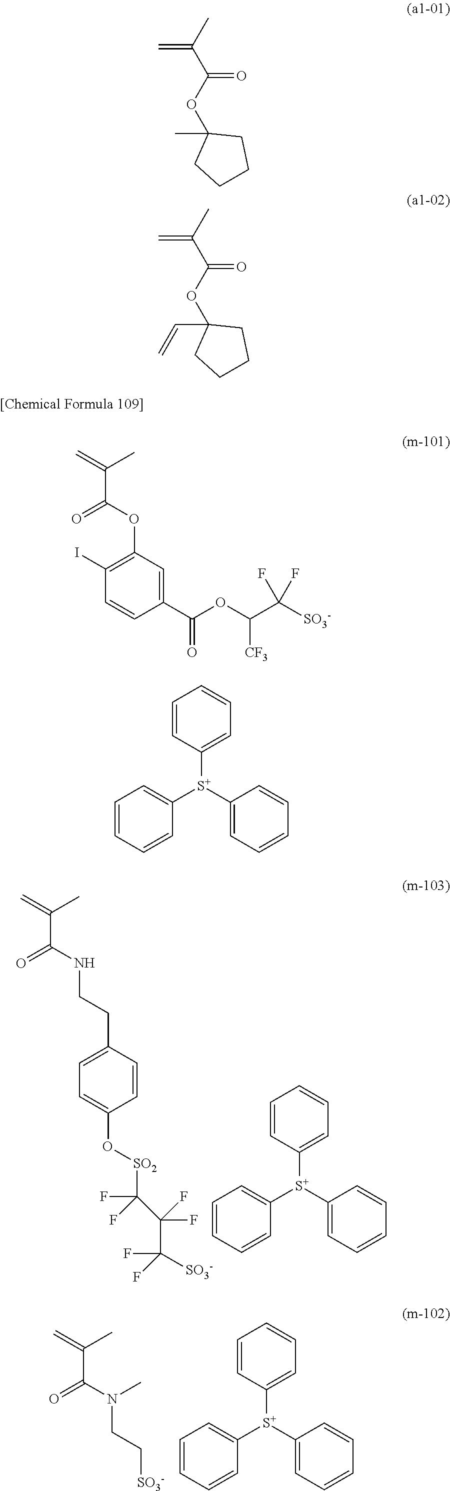

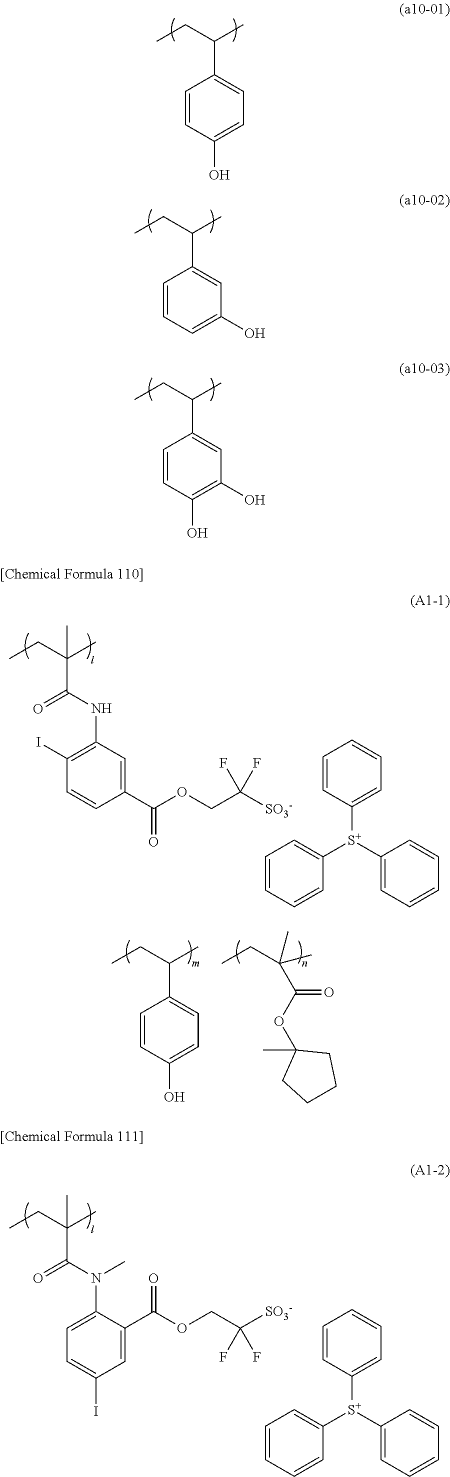

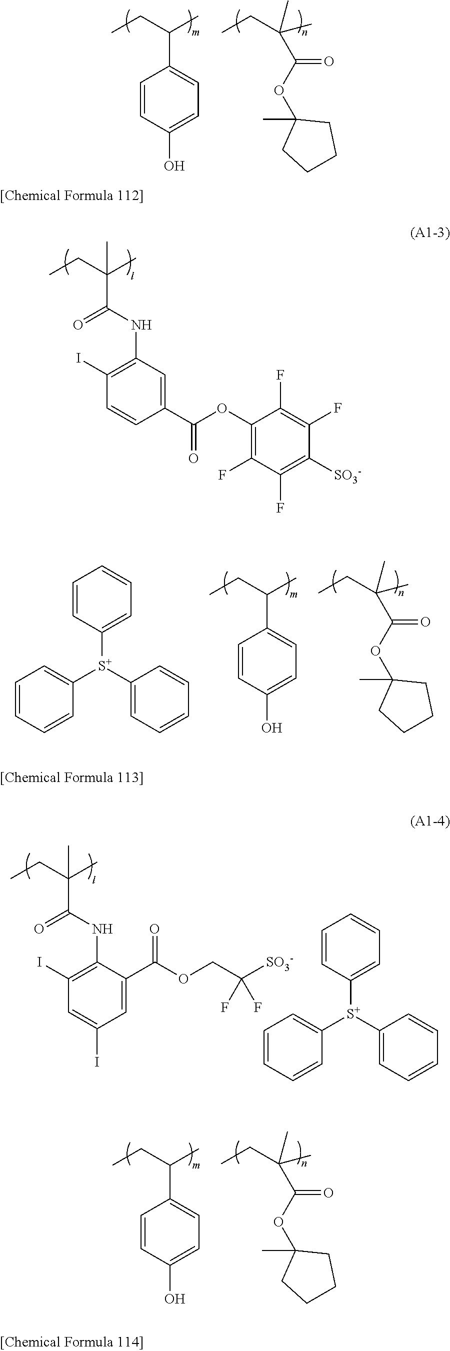

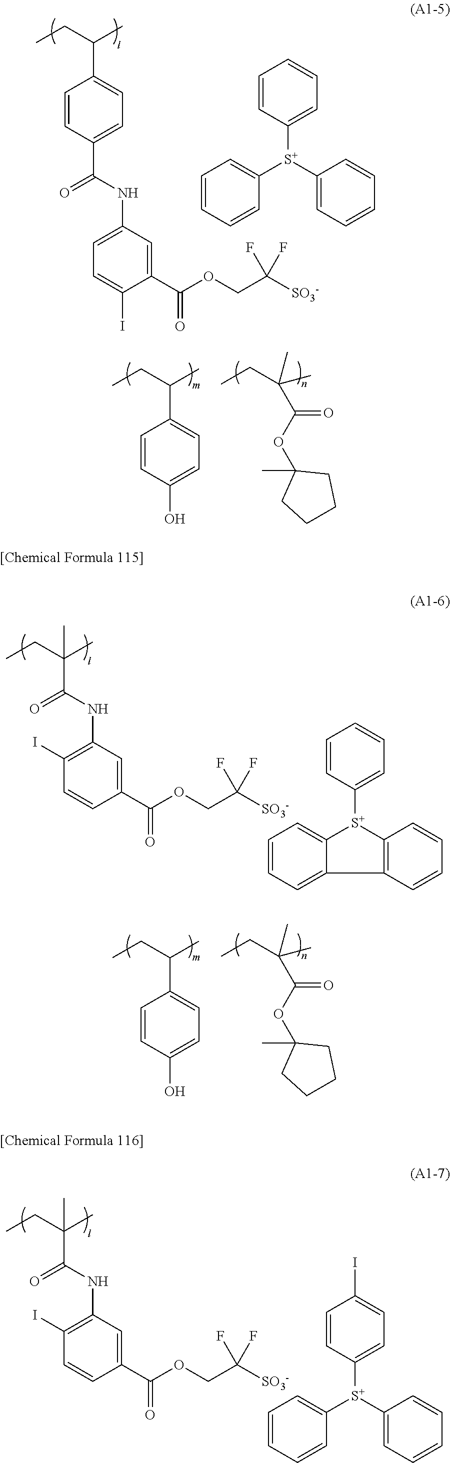

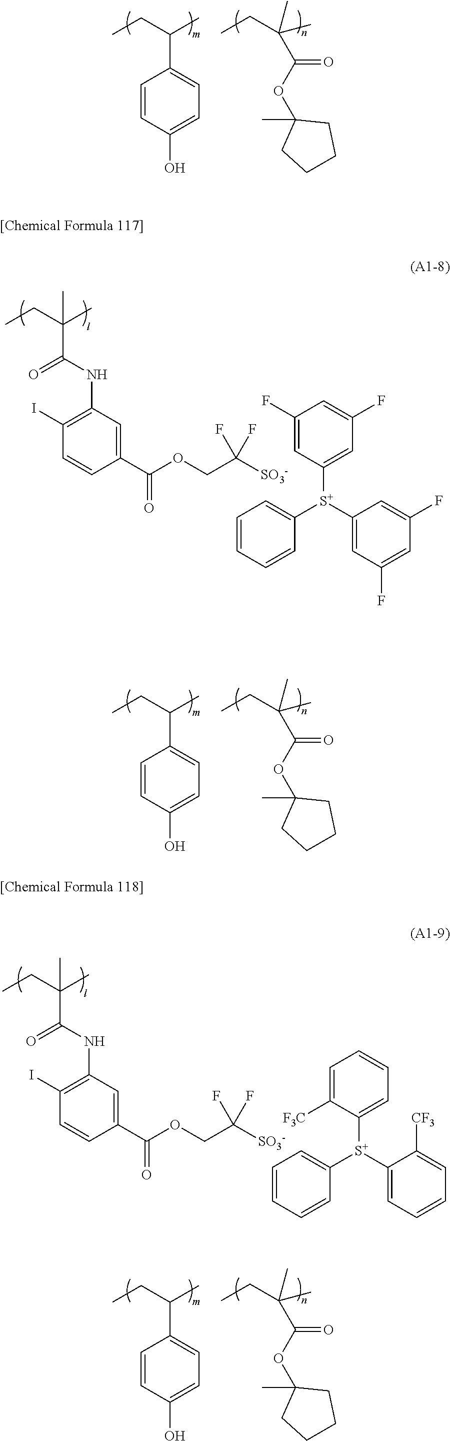

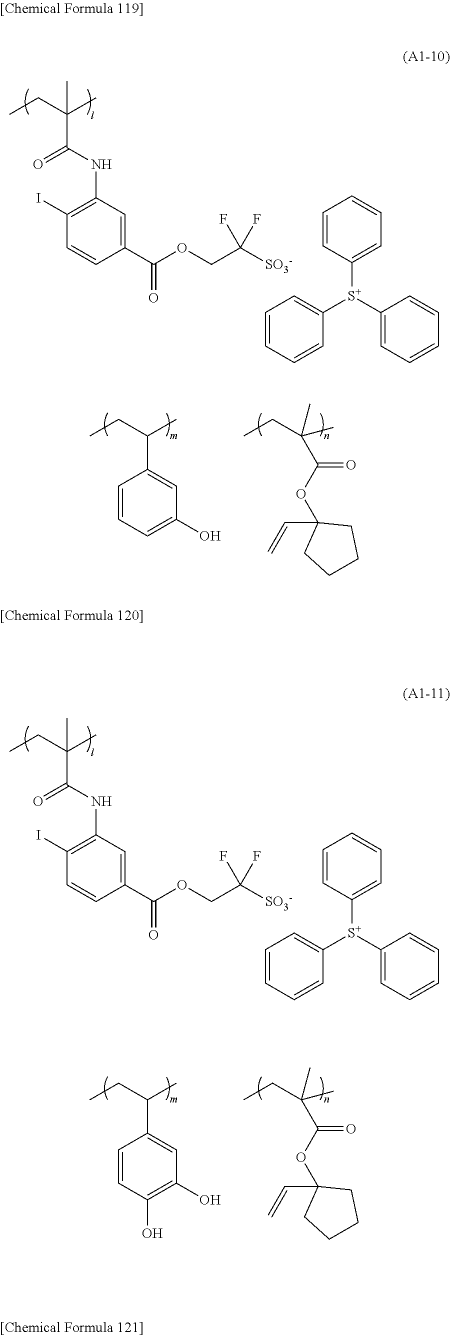

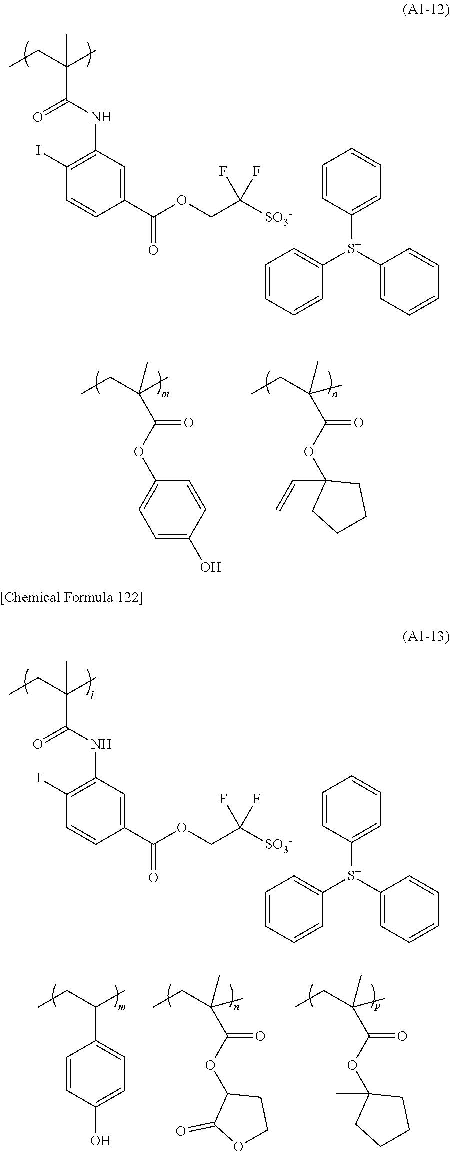

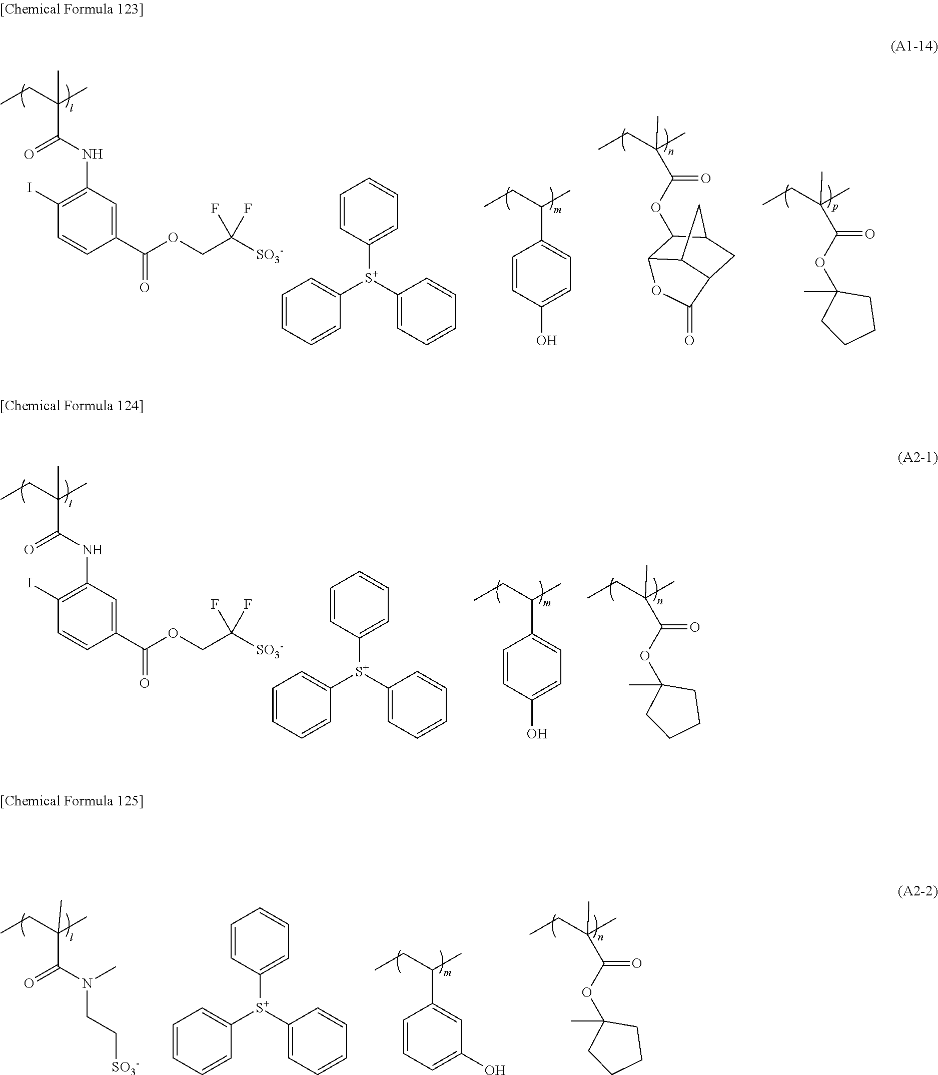

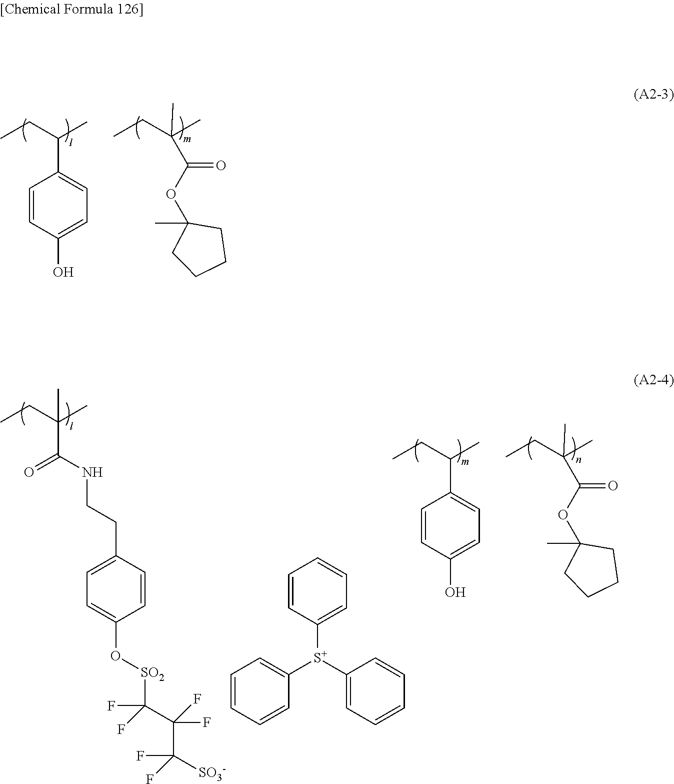

With respect to the obtained polymer compounds, the copolymer compositional ratio (the ratio (molar ratio) between constitutional units derived from compounds which are monomers) of the polymer compound, determined by ¹³C-NMR, and the weight-average molecular weight (Mw) and the polydispersity (Mw/Mn) in terms of standard polystyrene, determined by GPC measurement, are collectively listed in Table 1.

TABLE 1

| | Copolymer | | | |
|---|---|---|---|---|
| | composition | Composition | | |
| | (constitutional | ratio | | |
| | units derived | (proportion of | | |
| | from compounds | constitutional | | |
| Polymer | which are | units (molar | | |
| compound | each monomer) | ration)) | Mw | Mw/Mn |
| (A1-1) | (a0-m-1)/(a10- | 15/35/50 | 8400 | 1.65 |
| | 01pre)/(a1-01) | | | |
| (A1-2) | (a0-m-2)/(a10- | 15/35/50 | 7500 | 1.76 |
| | 01pre)/(a1-01) | | | |
| (A1-3) | (a0-m-3)/(a10- | 15/35/50 | 9300 | 1.68 |
| | 01pre)/(a1-01) | | | |
| (A1-4) | (a0-m-4)/(a10- | 15/35/50 | 8400 | 1.77 |
| | 01pre)/(a1-01) | | | |
| (A1-5) | (a0-m-5)/(a10- | 15/35/50 | 7700 | 1.73 |
| | 01pre)/(a1-01) | | | |
| (A1-6) | (a0-m-6)/(a10- | 15/35/50 | 8200 | 1.59 |
| | 01pre)/(a1-01) | | | |
| (A1-7) | (a0-m-7)/(a10- | 15/35/50 | 8400 | 1.71 |
| | 01pre)/(a1-01) | | | |
| (A1-8) | (a0-m-8)/(a10- | 15/35/50 | 7900 | 1.68 |
| | 03pre)/(a1-01) | | | |
| (A1-9) | (a0-m-9)/(a10- | 15/35/50 | $800 | 1.67 |
| | 01pre)/(a1-01) | | | |
| (A1-10) | (a0-m-1)/(a10- | 15/35/50 | 9200 | 1.65 |
| | 02pre)/(a1-02) | | | |
| (A1-11) | (a0-m-1/(a10- | 20/20/60 | 8400 | 1.69 |
| | 03pre)/(a1-02) | | | |
| (A1-12) | (a0-m-1)/(a10-04)/ | 20/30/50 | 8700 | 1.31 |
| | (a1-02) | | | |
| (A1-13) | (a0-m-1)/(a10-01pre)/ | 15/30/5/30 | 7900 | 1.69 |
| | (a2-01)/(a1-01) | | | |
| (A3-14) | (a0-m-1)/(a10-01pre)/ | 15/30/5/50 | 10200 | 1.76 |
| | (a2-02)/(a1-01) | | | |
| (A2-1) | (m-101)/(a10- | 15/35/50 | 8500 | 1.73 |
| | 01pre)/(a1-01) | | | |
| (A2-2) | (m-102)/(a10- | 15/35/50 | 9100 | 1.75 |
| | 02pre)/(a1-01) | | | |
| (A2-3) | (a10-01pre)/(a1-01) | 40/60 | 6800 | 1.55 |
| (A2-4) | (m-103)/(a10- | 15/35/50 | 7500 | 1.72 |
| | 01pre)/(a1-01) | | | |

### <Preparation of Resist Composition>

### Examples 1 to 18 and Comparative Examples 1 to 4

Each of the components shown in Table 2 was mixed and dissolved to prepare a resist composition of each Example.

TABLE 2

| | Compound | Compound | Compound | Compound |
|---|---|---|---|---|
| | (A) | (B) | (D) | (S) |
| Example 1 | (A)-1 | — | (D)-1 | (S)-1 |
| | [100] | | [6.0] | [6400] |
| Example 2 | (A)-2 | — | (D)-1 | (S)-1 |
| | [100] | | [6.0] | [6400] |
| Example 3 | (A)-3 | — | (D)-1 | (S)-1 |
| | [100] | | [6.0] | [6400] |
| Example 4 | (A)-4 | — | (D)-1 | (S)-1 |
| | [100] | | [6.0] | [6400] |
| Example 5 | (A)-5 | — | (D)-1 | (S)-1 |
| | [100] | | [6.0] | [6400] |
| Example 6 | (A)-6 | — | (D)-1 | (S)-1 |
| | [100] | | [6.0] | (6400] |
| Example 7 | (A)-7 | — | (D)-1 | (S)-1 |
| | [100] | | [6.0] | [6400] |
| Example 8 | (A)-8 | — | (D)-1 | (S)-1 |
| | [100] | | [6.0] | [6400] |
| Example 9 | (A)-9 | — | (D)-1 | (S)-1 |
| | [100] | | [6.0] | [6400] |
| Example 10 | (A)-10 | — | (D)-1 | (S)-1 |
| | [100] | | [6.0] | [6400] |
| Example 11 | (A)-11 | — | (D)-1 | (S)-1 |
| | [100] | | [6.0] | [6400] |
| Example 12 | (A)-12 | — | (D)-1 | (S)-1 |
| | [100] | | [6.0] | [6400] |
| Example 13 | (A)-13 | — | (D)-1 | (S)-1 |
| | [100] | | [6.0] | [6400] |
| Example 14 | (A)-14 | — | (D)-1 | (S)-1 |
| | [100] | | [6.0] | [6400) |
| Example 15 | (A)-1 | — | (D)-2 | (S)-1 |
| | [100] | | [7.0] | [6400] |
| Example 16 | (A)-1 | — | (D)-3 | (S)-1 |
| | (100) | | [9.8] | [6400] |
| Example 17 | (A)-1 | (B)-1 | (D)-1 | (S)-1 |
| | [100] | [4.0] | [6.5] | [6400] |
| Example 18 | (A)-1 | (B)-2 | (D)-1 | (S)-1 |
| | [100] | [6.2] | [6.5] | [6400] |
| Comparative | (A)-21 | — | (D)-1 | (S)-1 |
| Example 1 | [100] | | [6.0] | [6400] |
| Comparative | (A)-22 | — | (D)-1 | (S)-1 |
| Example 2 | [100] | | [6.0] | [6400] |
| Comparative | (A)-23 | (B)-3 | (D)-1 | (S)-1 |
| Example: 3 | [100] | [20] | [6.0] | [6400] |
| Comparative | (A)-24 | — | (D)-1 | (S)-1 |
| Example 4 | [100] | | [6.0] | [6400] |

In Table 2, each abbreviation has the following meaning. The numerical values in the brackets represent the blending amounts (parts by mass, in terms of solid content).
(A)-1 to (A)-14: polymer compounds (A1-1) to (A1-14) shown above
(A)-21 to (A)-24: polymer compounds (A2-1) to (A2-4) shown above
(B)-1: An acid generator consisting of the following compound (B-1)
(B)-2: An acid generator consisting of the following compound (B-2)
(B)-3: An acid generator consisting of the following compound (B-3)

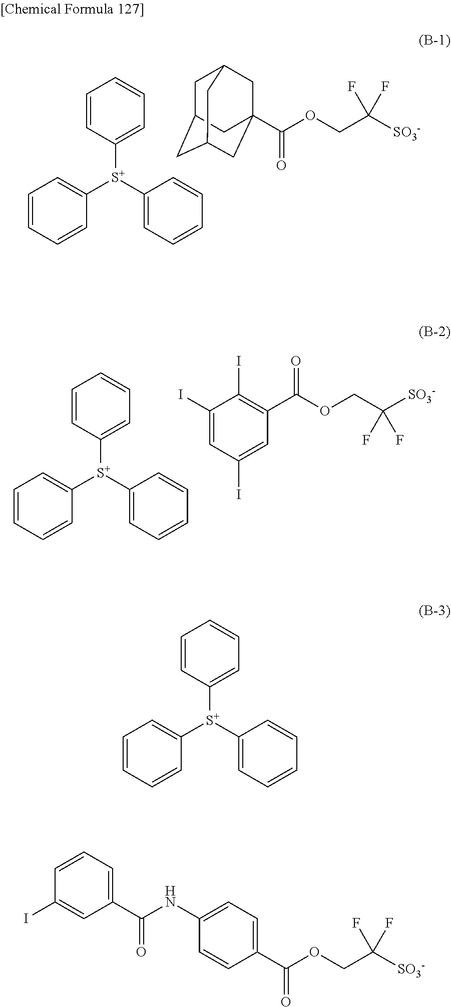

(D)-1: Acid generator consisting of the following compound (D1-1),
(D)-2: An acid generator comprising the following compound (D1-2).
(D)-3: An acid generator consisting of the following compound (D1-3).

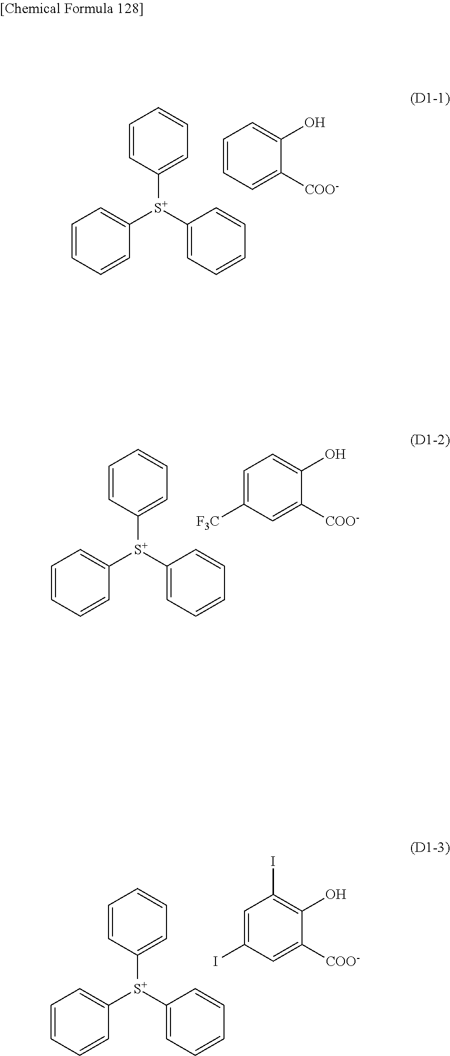

(S)-1: mixed solvent of propylene glycol monomethyl ether acetate/propylene glycol monomethyl ether at, mass ratio of 60/40

### <Resist Pattern Formation>

Step of forming resist film:

The resist composition of each example was applied onto an 8-inch silicon substrate which had been subjected to a hexamethyldisilazane (HMDS) treatment using a spinner, the coated film was subjected to a pre-bake (PAB) treatment on a hot plate at a temperature of 110° C. for 60 seconds so that the coated film was dried to form a resist film having a film thickness of 0.50 nm.

Step of exposing resist film to light:

Next, the resist film was subjected to drawing (light exposure) to obtain a contact hole pattern (hereinafter, referred to as “CH pattern”) in which holes having, as a target size, a diameter of 26 nm were arranged at equal intervals (pitch: 46 nm) by using an electron beam lithography apparatus JEOL JBX-9300FS (manufactured by JEOL Ltd.) at an acceleration voltage of 100 kV.

Thereafter, a post exposure bake (PEB) treatment was performed thereon at 100° C. for 60 seconds.

Step of developing resist film exposed to light:

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.).

Thereafter, water rinsing was carried out with pure water for 15 seconds.

As a result of the above, a CH pattern in which holes having a diameter of 26 nm were arranged at equal intervals (pitch: 46 nm) was formed.

### [Evaluation of Optimum Exposure Amount (Eop)]

According to the <Resist pattern formation>, an optimum exposure amount Eop (μC/cm²) for forming the CH pattern having the target size was determined. The results thereof are listed in the columns of “Eop (μC/cm²)” in Table 3.

[Evaluation of in-Plane Uniformity (CDU) of Pattern Dimensions]

The CH pattern formed according to the <Resist pattern formation> was observed from the upper side of the CH pattern, and the hole diameter (nm) of each of the holes was measured with a scanning electron microscope (CD-SEM, acceleration voltage: 500 V, trade name: CG5000, manufactured by Hitachi High-Tech Corporation). Then, the triple value (3σ) of the standard deviation (σ) calculated from the measurement result was determined. The obtained results are shown in Table 3 as “CDU (nm)”.

The lower the value of 3σ determined as described above is, the higher the critical dimension (CD) uniformity of the plurality of holes formed in the resist film is.

### [Evaluation of Light Exposure Margin (EL Margin)]

In the drawing (exposure) of the <Resist pattern formation>, the exposure amount where the hole width (size) of the CH pattern is 26 nm×105% is defined as E(+), and the exposure amount where the hole width (size) of the CH pattern is 26 nm×95% is defined as E(−). From the following equation, the light exposure margin of plus or minus 5% was calculated. The results are shown in Table 3 as “5% EL (%)”.

$$EL ( 5 \% ) = \{ E ( + ) \cdot E ( \cdot ) \} / Optimal exposure amount Eop \times 100$$

In this way, the obtained EL (5%) indicates that the larger its value, the smaller the dimensional change due to variations in exposure amount, and the wider the process margin.

TABLE 3

| | | | | | 5% |
|---|---|---|---|---|---|
| | PAB | PBB | Eop | CDU | EL |
| | (° C.) | (° C.) | (μC/cm²) | (nm) | (%) |
| Example 1 | 110 | 100 | 106 | 4.3 | 8.7 |
| Example 2 | 110 | 100 | 107 | 4.1 | 9.0 |
| Example 3 | 110 | 100 | 106 | 4.4 | 8.8 |
| Example 4 | 110 | 100 | 98 | 3.8 | 8.9 |
| Example 5 | 110 | 100 | 104 | 3.9 | 9.0 |
| Example ? | 110 | 100 | 107 | 4.2 | 8.8 |
| Example 7 | 110 | 100 | 101 | 4.0 | 8.8 |
| Example 8 | 110 | 100 | 98 | 3.8 | 8.7 |
| Example 9 | 110 | 100 | 100 | 4.1 | 8.8 |
| Example 10 | 110 | 100 | 108 | 4.2 | 8.8 |
| Example 11 | 110 | 100 | 102 | 4.4 | 8.6 |
| Example 12 | 110 | 100 | 103 | 4.3 | 8.5 |
| Example 13 | 110 | 100 | 106 | 4.5 | 8.7 |
| Example 14 | 110 | 100 | 105 | 4.3 | 8.7 |
| Example 15 | 110 | 100 | 106 | 4.2 | 8.8 |
| Example 16 | 110 | 100 | 98 | 3.8 | 8.7 |
| Example 17 | 110 | 100 | 104 | 4.5 | 8.4 |
| Example 18 | 110 | 100 | 97 | 4.1 | 8.5 |
| Comparative | 110 | 100 | 109 | 5.5 | 7.5 |
| Example 1 | | | | | |
| Comparative | 110 | 100 | 136 | 6.2 | 7.4 |
| Example 2 | | | | | |
| Comparative | 110 | 100 | 112 | 5.8 | 7.2 |
| Example 3 | | | | | |
| Comparative | 110 | 100 | 122 | 4.6 | 7.7 |
| Example 4 | | | | | |

From the results shown in Table 3, the resist compositions of Examples 1 to 18, to which the present invention was applied, exhibited good sensitivity. CDU, and EL margin.

In contrast, the resist composition of Comparative Example 1, which is outside the scope of the present invention, exhibited inferior CDU. The resist composition of Comparative Example 2 showed both inferior sensitivity and CDU. The resist composition of Comparative Example 3 exhibited inferior CDU, and the resist composition of Comparative Example 4 showed inferior sensitivity.

In addition, the resist compositions of Examples 1 to 18 had a higher 5% EL (%) value, which is an indicator of light exposure margin, compared to the resist compositions of Comparative Examples 1 to 4.

Based on the above, according to the resist composition of the present invention, it has been confirmed that high sensitivity when forming a resist pattern and improvements in lithography characteristics such as dimensional uniformity am achieved, and that the process margin is wide.

Hereinbefore, preferred examples of the present invention have been described, but the present invention is not limited to these examples. Additions, omissions, substitutions, and other modifications can be made without departing from the scope of the present invention. The present invention is not limited by the description above, but is limited only by the appended claims.

What is claimed is:

1. A resist composition which generates an acid upon light exposure and whose solubility in a developing solution is changed by an action of the acid, the resist composition comprising:
a resin component (A1) whose solubility in a developing solution is changed by the action of the acid,
wherein the resin component (A1) has a constitutional unit (a0) derived from a compound represented by General Formula (a0-m), 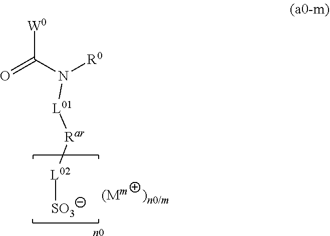
wherein W⁰ represents a polymerizable group-containing group, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as an atomic valence allows, m represents an integer of 1 or more, and (M^{m+})_{n0/m} represents a counter cation.

2. The resist composition according to claim 1,
wherein the constitutional unit (a0) is a constitutional unit represented by General Formula (a0-u1), 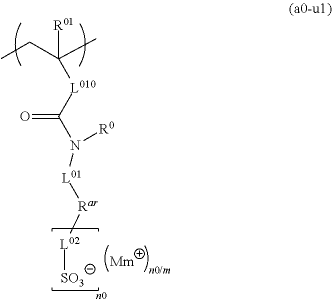
wherein R⁰¹ is an alkyl group having 1 to 5 carbon atoms, a halogenated alkyl group having 1 to 5 carbon atoms, a halogen atom, or a hydrogen atom, L⁰¹⁰ represents a divalent linking group or a single bond, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as the atomic valence allows, m represents an integer of 1 or more, and (M^{m+})_{n0/m} represents a counter cation.

3. The resist composition according to claim 1,
wherein the constitutional unit (a0) is a constitutional unit represented by General Formula (a0-u2), 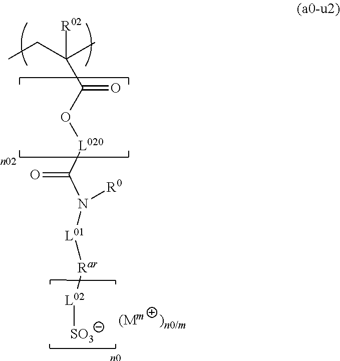
wherein R⁰² is an alkyl group having 1 to 5 carbon atoms, a halogenated alkyl group having 1 to 5 carbon atoms, a halogen atom, or a hydrogen atom, L⁰²⁰ represents a divalent linking group, n02 is an integer from 0 to 2, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as the atomic valence allows, m represents an integer of 1 or more, and (M^{m+})_{n0/m} represents a counter cation.

4. A resist pattern formation method comprising:
forming a resist film on a support using the resist composition according to claim 1;
exposing the resist film to light; and
developing the resist film exposed to light to form a resist pattern.

5. A compound represented by General Formula (a0-m), 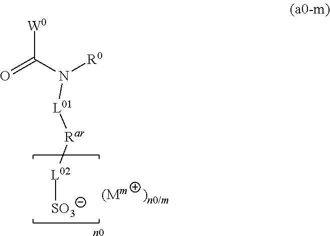
wherein W⁰ represents a polymerizable group-containing group, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as the atomic valence allows, m represents an integer of 1 or more, and (M^{m+})_{n0/m} represents a counter cation.

6. The compound according to claim 5, wherein the compound is represented by General Formula (a0-m1), 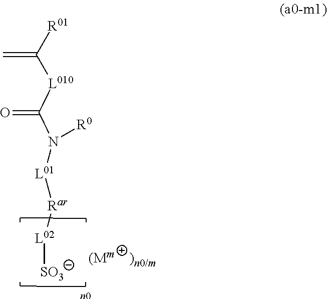
wherein R⁰¹ is an alkyl group having 1 to 5 carbon atoms, a halogenated alkyl group having 1 to 5 carbon atoms, a halogen atom, or a hydrogen atom, L⁰¹⁰ represents a divalent linking group or a single bond, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as the atomic valence allows, m represents an integer of 1 or more, and (M^{m+})_{n0/m} represents a counter cation.

7. A compound according to claim 5, wherein the compound is represented by General Formula (a0-m2), 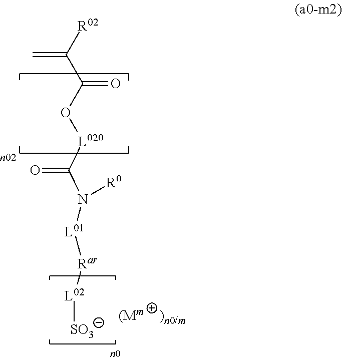
wherein R⁰² is an alkyl group having 1 to 5 carbon atoms, a halogenated alkyl group having 1 to 5 carbon atoms, a halogen atom, or a hydrogen atom, L⁰²⁰ represents a divalent linking group, n02 represents an integer from 0 to 2, R⁰ represents a hydrocarbon group, an acyl group, an alkoxyalkyl group, or a hydrogen atom, L⁰¹ represents a divalent linking group or a single bond, R^{ar} represents an aromatic ring to which an iodine atom is bonded as a substituent, a substituent other than the iodine atom may be bonded to the aromatic ring, L⁰² represents a divalent linking group, n0 represents an integer of 1 or more, as long as the atomic valence allows, m represents an integer of 1 or more, and (M^{m+})_{n0/m} represents a counter cation.

8. A polymer compound which has a constitutional unit derived from the compound according to claim 5.
